(19) United States
(12) Patent Application Publication
(10) Pub. No.: US 2024/0299295 A1
(43) Pub. Date: Sep. 12, 2024

(54) AQUEOUS FORMULATIONS OF TOFACITINIB AND TOFACITINIB SALTS

(71) Applicant: BIORA THERAPEUTICS, INC, San Diego, CA (US)

(72) Inventors: Thotsaphon VONGASAVARIT, San Diego, CA (US); Aruna PERERA, Alameda, CA (US); Kevin David HOWE, San Diego, CA (US)

(21) Appl. No.: 18/578,472

(22) PCT Filed: Sep. 9, 2022
(86) PCT No.: PCT/US2022/043136

§ 371 Date: Jan. 11, 2024

Related U.S. Application Data
(60) Provisional application No. 63/242,436, filed on Sep. 9, 2021.

(51) Int. Cl. A61K 9/00; A61K 31/519; A61K 47/32; A61K 47/40

(52) U.S. Cl. CPC A61K 9/0095; A61K 31/519; A61K 47/32; A61K 47/40

(57) ABSTRACT

Aqueous formulations containing active pharmaceutical ingredient and cyclodextrin and their method of preparation and their uses are provided herein

### BACKGROUND

Tofacitinib citrate (Xeljanz®), an oral, systemically available, pan-JAK inhibitor, was approved in the United States in November, 2012 to treat adults with moderately to severely active rheumatoid arthritis who have had an inadequate response to, or who are intolerant of, methotrexate. JAK enzymes transmit cytokine signaling through pairing of JAKs (e.g., JAK1/JAK3, JAK1/JAK2, JAK1/TyK2, and JAK2/JAK2). Tofacitinib inhibited the in vitro activities of JAK1/JAK2, JAK1/JAK3, and JAK2/JAK2 combinations with IC₅₀ of 406, 56, and 1377 nM, respectively. It has been investigated as an immunosuppressive agent for the therapy of several conditions such as organ transplants, xeno transplantation, lupus, multiple sclerosis, rheumatoid arthritis, psoriasis, Type I diabetes and complications from diabetes, cancer, asthma, atopic dermatitis, autoimmune thyroid disorders, ulcerative colitis, Crohn's disease, Alzheimer's disease, leukemia and other indications, where immunosuppression would be desirable (see WO 03/048126). In the US and Europe, oral dosage forms of tofacitinib citrate (XELJANZ®, JAKVINUS®, tasocitinib citrate, CP-690550 citrate) have been approved for use in the treatment of rheumatoid arthritis, psoriatic arthritis and ulcerative colitis (UC), with some limitations.

### SUMMARY

Embodiments disclosed herein address the aforementioned demands by providing aqueous formulations of tofacitinib, method of preparation of such aqueous formulation and their uses.

The disclosure, at least in part, provides an aqueous formulation comprising tofacitinib or pharmaceutically acceptable salt of tofacitinib or tofacitinib free base and cyclodextrin. In some embodiments, the aqueous formulation comprises at least about 10% w/w of cyclodextrin. In some embodiments, after extended period of time under storage condition, at least about 75% of the tofacitinib remains in the aqueous formulation. In some embodiments, at least about 80% of the tofacitinib remains in the aqueous formulation after extended period of time under storage condition. In some embodiments, at least about 85% of the tofacitinib remains in the aqueous formulation after extended period of time under storage condition. In some embodiments, at least about 90% of the tofacitinib remains in the aqueous formulation after extended period of time under storage condition. In some embodiments, at least about 93% of the tofacitinib remains in the aqueous formulation after extended period of time under storage condition. In some embodiments, at least about 95% of the tofacitinib remains in the aqueous formulation after extended period of time under storage condition. In some embodiments, at least about 97% of the tofacitinib remains in the aqueous formulation after extended period of time under storage condition. In some embodiments, at least about 98% of the tofacitinib remains in the aqueous formulation after extended period of time under storage condition. In some embodiments, at least about 99% of the tofacitinib remains in the aqueous formulation after extended period of time under storage condition.

In some embodiments, the extended period of time is about 1 week. In some embodiments, the extended period of time is about 2 weeks. In some embodiments, the extended period of time is about 3 weeks. In some embodiments, the extended period of time is about 1 month. In some embodiments, the extended period of time is about 2 months. In some embodiments, the extended period of time is about 3 months. In some embodiments, the extended period of time is about 4 months. In some embodiments, the extended period of time is about 5 months. In some embodiments, the extended period of time is about 6 months. In some embodiments, the extended period of time is about 8 months. In some embodiments, the extended period of time is about 10 months. In some embodiments, the extended period of time is about 12 months. In some embodiments, the extended period of time is about 15 months. In some embodiments, the extended period of time is about 18 months. In some embodiments, the extended period of time is about 21 months. In some embodiments, the extended period of time is about 24 months.

In some embodiments, the storage condition has a storage temperature of about 2-8° C. In some embodiments, the storage condition has a storage temperature of about 5° C. In some embodiments, the storage condition has a storage temperature of about 25° C. In some embodiments, the storage condition has a storage temperature of about 25° C. and a relative humidity of about 65%. In some embodiments, he storage condition has a storage temperature of about 40° C. and a relative humidity of about 75%.

In one exemplary embodiment, the aqueous formulation can comprise a pharmaceutically acceptable salt of tofacitinib or tofacitinib free base and cyclodextrin. In one aspect of this embodiment, the tofacitinib salt form can be an acid addition salt. In another aspect of this embodiment, the tofacitinib salt can be selected from the group consisting of tofacitinib aceglutamate, acephyllinate, acetamidobenzoate, 2-acetamidobenzoate, 4-acetamidobenzoate, acetate, acetylasparaginate, acetylaspartate, acid citrate, adipate, acetylsalicylate, aminosalicylate, anhydromethylenecitrate, ascorbate, aspartate, benzenesulfonate (besylate), benzoate, bicarbonate, bisulfate, bitartrate, borate, bromide, butanoate, caffeate, caftarate, camphorate (+-camphorate), camsylate (camsilate, camphorsulfonic acid), carbonate, cholate, chloride, chlorogenate, chlorophenoxyacetate, cis-cinnamate, trans-cinnamate, citrate, closylate (4-chlorobenzene sulfonate), cromesilate (6,7-Dihydroxycourmarin-4-methanesulfonate), cyclamate, cyclohexanecarboxylate, decanoate (caprate), dehydrocholate, 2,5-dihydroxybenzoate (gentisate), edetate (EDTA), edisylate (1,2-ethanedisulfonate), erythorbate (isoascorbate), estolate (laurylsulfate), esylate (ethanesulfonate), ethylsulfate, fendizoate, ferulate, formate, fumarate, gluceptate, glucoheptanoate (2,3,4,5,6,7-hexahydroxyheptanoic acid), gluconate (D-gluconate), glucuronate, glutamate, glutarate, glycerophosphate, glycinate, glycolate, glycyrrhizate, glyoxylate (oxaldehydate), hexanoate, hippurate, hydrobromide, hydrochloride, hydroiodide, 4-hydroxybenzenesulfonate, 3-hydroxybenzoate, 4-hydroxybenzoate, hydroxynaphthoate, 3-hydroxybutanoate, 4-hydroxybutanoate, 4-hydroxycinnamate (p-coumarate), 3-hydroxypropionate, iodide, isethionate (2-hydroxyethanesulfonate), isopropyl mesylate (isopropyl methanesulfonate), lactate (D,L-lactate), lactobionate (erythromycin mono (4-0-β-D-galactopyranosyl-D-gluconate)), laurate, lipoate ((R)-lipoate), lysine, malate (2-hydroxybutanedioic acid), maleate ((Z)-but-2-enedioate), mandelate (2-hydroxy-2-phenylacetate), mesotartarate, mesylate (methanesulfonate), metaphosphate, 4-methylpentanoate, 2-methylpropionate, methylsuccinate, methylsulfate, mucate (galactarate; (2S,3R,4S,5R)-2,3,4,5-tetrahydroxyhexanedioate), napadisylate (1,5-naphthalenedisulfonate), napsylate (naphthalene-2-sulfonate), nicotinate, nitrate, octanoate (caprylate), oleate, orotate, oxalate (ethanedioate), oxoglurate, palmitate, pamoate (3-carboxy-1-[(3-carboxy-2-oxidonaphthalen-1-yl)methyl]naphthalen-2-olate), pantothenate (Vitamin B5), pectinate, pentanoate, phenylacetate, phenylethylbarbiturate, phosphate, picrate, pimelate (heptanedioate), polygalacturonate (pectate), polyglutamate, propionate (propanoate), pyridoxal phosphate, L-pyroglutamate, pyruvate, quinate, saccharate, saccharinate, salicylate, sebacate, sinapate, sorbate, stearate, stearylsulfate, succinate, sulfate, sulfosalicylate, tannate, tartrate, teoclate, terephthalate, thiocyanate, tosylate (toluene 4-sulfonate), trans-coutarate, trimethylacetate, undecanoate (undecylate), urate and xinafoate (1-hydroxy-2-naphthoate).

In one aspect of this embodiment, the concentration of acceptable salt of tofacitinib or tofacitinib free base can be at least about 5 mg/mL, at least about 10 mg/mL, at least about 20 mg/mL, at least about 30 mg/mL, at least about 40 mg/mL, at least about 50 mg/mL, at least about 60 mg/mL, at least about 70 mg/mL, at least about 80 mg/mL, at least about 90 mg/mL, at least about 100 mg/mL, at least about 110 mg/mL, at least about 120 mg/mL, at least about 130 mg/mL, at least about 140 mg/mL, at least about 150 mg/mL, at least about 160 mg/mL, at least about 170 mg/mL, at least about 180 mg/mL, at least about 190 mg/mL, or at least about 200 mg/mL. In another aspect of this embodiment, the tofacitinib salt can be tofacitinib citrate, and the tofacitinib citrate concentration can be at least about 20 mg/mL, at least about 30 mg/mL, at least about 40 mg/mL, at least about 50 mg/mL, at least about 60 mg/mL, at least about 70 mg/mL, at least about 80 mg/mL, at least about 90 mg/mL, at least about 100 mg/mL, at least about 110 mg/mL, at least about 120 mg/mL, at least about 130 mg/mL, at least about 140 mg/mL, at least about 150 mg/mL, at least about 160 mg/mL, at least about 170 mg/mL, at least about 180 mg/mL, at least about 190 mg/mL, or at least about 200 mg/mL.

In one aspect of this embodiment, the cyclodextrin (CD) can be a natural CD. In a specific aspect of this embodiment, the natural CD can be α-CD, β-CD, or γ-CD. In one aspect of this embodiment, the cyclodextrin (CD) can be a modified CD. In a specific aspect of this embodiment, the modified CD can be 2-hydroxypropyl-α-CD, 2-hydroxypropyl-β-CD, 2-hydroxypropyl-γ-CD, sulfobutylether β-CD, sulfobutylether γ-cyclodextrin, dimethyl-α-CD, trimethyl-αCD, methyl-β-CD, carboxymethyl-β-CD, dimethyl-β-CD; trimethyl-β-CD; hydroxyethyl-βCD, dimethyl-γ-CD, trimethyl-γ-CD, hydroxypropyl-γ-CD, sulfobutylether-γCD sodium salt, or sugammadex. In yet another aspect of this embodiment, the cyclodextrin can be a branched CD. In a specific aspect of this embodiment, the branched CD can be glucosyl-βCD, maltosyl-βCD, and glucoronyl-glucosyl-βCD. In one aspect of this embodiment, the cyclodextrin (CD) can be commercially available Kelptose® HP (0.90 molecular substitution).

In one aspect of this embodiment, the amount of cyclodextrin in the aqueous formulation can range from about 10% to about 90%, more particularly about 25% to about 80%, even more particularly about 35% to about 75%, or even more particularly about 55% to about 70% based on the weight of the aqueous formulation. In another aspect of this embodiment, the amount of cyclodextrin in the aqueous formulation can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90% based on the weight of the aqueous formulation.

In one aspect of this embodiment, the aqueous formulation can further comprise a stabilizer. In a specific aspect of this embodiment, the stabilizer can be a polymer. In another specific aspect of this embodiment, the polymer can be a polyacrylic acid polymer. In some aspects, the polyacrylic acid polymer can be a crosslinked polyacrylic acid polymer. In some aspects, the polyacrylic acid polymer can be a homopolymer of acrylic acid crosslinked with allyl sucrose or allyl pentaerythritol. In yet another specific aspect, the crosslinked polyacrylic acid polymer can be a Carbomer. In still another specific aspect, the Carbomer may be selected from the group consisting of Carbomer homopolymer type A, Carbomer homopolymer type B, Carbomer homopolymer type C, Carbomer copolymer, Carbomer interpolymer, or a combination thereof. In some other aspects, the polyacrylic acid polymer can be a Carbopol® copolymer. In some further aspects, the Carbopol® copolymer can be a copolymer of an acrylic acid and a C₁₀-C₃₀ alkyl acrylate crosslinked with allyl pentaerythritol. In some other aspects, the polyacrylic acid polymer can be a Carbopol® interpolymer. In some further aspects, the Carbopol® interpolymer can be a Carbopol homopolymer or copolymer containing a block copolymer of polyethylene glycol and a long chain alkyl acid ester. In a specific aspect of this embodiment, the stabilizer can be selected from a group consisting of Carbopol® 71G, Carbopol® 971P, Carbopol® 974P, Carbopol® 980, Carbopol® 981, Carbopol® 5984, Carbopol® 934, Carbopol® 934P, Carbopol® 940; Carbopol® 941; Carbopol 950, Carbopol 980, Carbopol 951 and Carbopol 981, Carbopol® 5984, and a combination of any two or more of the foregoing.

In one aspect of this embodiment, the concentration of the stabilizer in the aqueous formulation can range from about 0.05% w/w to about 3.0 w/w, from about 0.1% w/w to about 2.5% w/w, from about 0.25% w/w to about 2.0% w/w, or from about 0.5% w/w to about 1.5% w/w.

In one aspect of this embodiment, the pH of the aqueous formulation can range from about 3.6 to about 8.0. In another aspect of this embodiment, the pH of the aqueous formulation can be about 3.6, about 3.7, about 3.8, about 3.9, about 4, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0. In a specific exemplary embodiment, the pH of the aqueous formulation can be in the range of about 4.5 to about 7.5, about 5 to about 8, or about 5 to about 7.

In one exemplary embodiment, the aqueous formulation can comprise a pharmaceutically acceptable salt of tofacitinib or tofacitinib free base and hydroxypropyl-β-cyclodextrin. In one aspect of this embodiment, the tofacitinib salt form can be an acid addition salt. In another aspect of this embodiment, the tofacitinib salt can be selected from the group consisting of tofacitinib aceglutamate, acephyllinate, acetamidobenzoate, 2-acetamidobenzoate, 4-acetamidobenzoate, acetate, acetylasparaginate, acetylaspartate, acid citrate, adipate, acetylsalicylate, aminosalicylate, anhydromethylenecitrate, ascorbate, aspartate, benzenesulfonate (besylate), benzoate, bicarbonate, bisulfate, bitartrate, borate, bromide, butanoate, caffeate, caftarate, camphorate (+-camphorate), camsylate (camsilate, camphorsulfonic acid), carbonate, cholate, chloride, chlorogenate, chlorophenoxyacetate, cis-cinnamate, trans-cinnamate, citrate, closylate (4-chlorobenzene sulfonate), cromesilate (6,7-Dihydroxycourmarin-4-methanesulfonate), cyclamate, cyclohexanecarboxylate, decanoate (caprate), dehydrocholate, 2,5-dihydroxybenzoate (gentisate), edetate (EDTA), edisylate (1,2-ethanedisulfonate), erythorbate (isoascorbate), estolate (laurylsulfate), esylate (ethanesulfonate), ethylsulfate, fendizoate, ferulate, formate, fumarate, gluceptate, glucoheptanoate (2,3,4,5,6,7-hexahydroxyheptanoic acid), gluconate (D-gluconate), glucuronate, glutamate, glutarate, glycerophosphate, glycinate, glycolate, glycyrrhizate, glyoxylate (oxaldehydate), hexanoate, hippurate, hydrobromide, hydrochloride, hydroiodide, 4-hydroxybenzenesulfonate, 3-hydroxybenzoate, 4-hydroxybenzoate, hydroxynaphthoate, 3-hydroxybutanoate, 4-hydroxybutanoate, 4-hydroxycinnamate (p-coumarate), 3-hydroxypropionate, iodide, isethionate (2-hydroxyethanesulfonate), isopropyl mesylate (isopropyl methanesulfonate), lactate (D,L-lactate), lactobionate (erythromycin mono (4-0-β-D-galactopyranosyl-D-gluconate)), laurate, lipoate ((R)-lipoate), lysine, malate (2-hydroxybutanedioic acid), maleate ((Z)-but-2-enedioate), mandelate (2-hydroxy-2-phenylacetate), mesotartarate, mesylate (methanesulfonate), metaphosphate, 4-methylpentanoate, 2-methylpropionate, methylsuccinate, methylsulfate, mucate (galactarate; (2S,3R,4S,5R)-2,3,4,5-tetrahydroxyhexanedioate), napadisylate (1,5-naphthalenedisulfonate), napsylate (naphthalene-2-sulfonate), nicotinate, nitrate, octanoate (caprylate), oleate, orotate, oxalate (ethanedioate), oxoglurate, palmitate, pamoate (3-carboxy-1-[(3-carboxy-2-oxidonaphthalen-1-yl)methyl]naphthalen-2-olate), pantothenate (Vitamin B5), pectinate, pentanoate, phenylacetate, phenylethylbarbiturate, phosphate, picrate, pimelate (heptanedioate), polygalacturonate (pectate), polyglutamate, propionate (propanoate), pyridoxal phosphate, L-pyroglutamate, pyruvate, quinate, saccharate, saccharinate, salicylate, sebacate, sinapate, sorbate, stearate, stearylsulfate, succinate, sulfate, sulfosalicylate, tannate, tartrate, teoclate, terephthalate, thiocyanate, tosylate (toluene 4-sulfonate), trans-coutarate, trimethylacetate, undecanoate (undecylate), urate and xinafoate (1-hydroxy-2-naphthoate).

In one aspect of this embodiment, the amount of hydroxypropyl-β-cyclodextrin in the aqueous formulation can range from about 10% to about 90%, particularly about 20% to about 60%, more particularly about 40 to about 60% based on the weight of the aqueous formulation. In another aspect of this embodiment, the amount of hydroxypropyl-β-cyclodextrin in the aqueous formulation can be greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, or greater than about 90%.

In one aspect of this embodiment, the concentration of acceptable salt of tofacitinib or tofacitinib free base can be at least about 5 mg/mL, at least about 10 mg/mL, at least about 20 mg/mL, at least about 30 mg/mL, at least about 40 mg/mL, at least about 50 mg/mL, at least about 60 mg/mL, at least about 70 mg/mL, at least about 80 mg/mL, at least about 90 mg/mL, at least about 100 mg/mL, at least about 110 mg/mL, at least about 120 mg/mL, at least about 130 mg/mL, at least about 140 mg/mL, at least about 150 mg/mL, at least about 160 mg/mL, at least about 170 mg/mL, at least about 180 mg/mL, at least about 190 mg/mL, or at least about 200 mg/mL. In another aspect of this embodiment, the tofacitinib salt can be tofacitinib citrate, and the tofacitinib citrate concentration can be at least about 20 mg/mL, at least about 30 mg/mL, at least about 40 mg/mL, at least about 50 mg/mL, at least about 60 mg/mL, at least about 70 mg/mL, at least about 80 mg/mL, at least about 90 mg/mL, at least about 100 mg/mL, at least about 110 mg/mL, at least about 120 mg/mL, at least about 130 mg/mL, at least about 140 mg/mL, at least about 150 mg/mL, at least about 160 mg/mL, at least about 170 mg/mL, at least about 180 mg/mL, at least about 190 mg/mL, or at least about 200 mg/mL.

In one aspect of this embodiment, the aqueous formulation can further comprise a stabilizer. In a specific aspect of this embodiment, the stabilizer can be a polymer. In another specific aspect of this embodiment, the polymer can be a polyacrylic acid polymer. In some aspects, the polyacrylic acid polymer can be a crosslinked polyacrylic acid polymer. In some aspects, the polyacrylic acid polymer can be a homopolymer of acrylic acid crosslinked with allyl sucrose or allyl pentaerythritol. In yet another specific aspect, the crosslinked polyacrylic acid polymer can be a Carbomer. In still another specific aspect, the Carbomer may be selected from the group consisting of Carbomer homopolymer type A, Carbomer homopolymer type B, Carbomer homopolymer type C, Carbomer copolymer, Carbomer interpolymer, or a combination thereof. In some other aspects, the polyacrylic acid polymer can be a Carbopol® copolymer. In some further aspects, the Carbopol® copolymer can be a copolymer of an acrylic acid and a C₁₀-C₃₀ alkyl acrylate crosslinked with allyl pentaerythritol. In some other aspects, the polyacrylic acid polymer can be a Carbopol® interpolymer. In some further aspects, the Carbopol® interpolymer can be a Carbopol homopolymer or copolymer containing a block copolymer of polyethylene glycol and a long chain alkyl acid ester. In a specific aspect of this embodiment, the stabilizer can be selected from a group consisting of Carbopol® 71G, Carbopol® 971P, Carbopol® 974P, Carbopol® 980, Carbopol® 981, Carbopol® 5984, Carbopol® 934, Carbopol® 934P, Carbopol® 940; Carbopol® 941; Carbopol 950, Carbopol 980, Carbopol 951 and Carbopol 981, Carbopol® 5984, and a combination of any two or more of the foregoing.

In one aspect of this embodiment, the concentration of the stabilizer in the aqueous formulation can range from about 0.05% w/w to about 3.0 w/w, from about 0.1% w/w to about 2.5% w/w, from about 0.25% w/w to about 2.0% w/w, or from about 0.5% w/w to about 1.5% w/w.

In one aspect of this embodiment, the pH of the aqueous formulation can range from about 3.6 to about 8.0. In another aspect of this embodiment, the pH of the aqueous formulation can be about 3.6, about 3.7, about 3.8, about 3.9, about 4, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0. In a specific exemplary embodiment, the pH of the aqueous formulation can be in the range of about 4.5 to about 7.5, about 5 to about 8, or about 5 to about 7.

In one exemplary embodiment, the aqueous formulation can comprise a pharmaceutically acceptable salt of tofacitinib or tofacitinib free base and methyl-β-cyclodextrin. In one aspect of this embodiment, the tofacitinib salt form can be an acid addition salt. In another aspect of this embodiment, the tofacitinib salt can be selected from the group consisting of tofacitinib aceglutamate, acephyllinate, acetamidobenzoate, 2-acetamidobenzoate, 4-acetamidobenzoate, acetate, acetylasparaginate, acetylaspartate, acid citrate, adipate, acetylsalicylate, aminosalicylate, anhydromethylenecitrate, ascorbate, aspartate, benzenesulfonate (besylate), benzoate, bicarbonate, bisulfate, bitartrate, borate, bromide, butanoate, caffeate, caftarate, camphorate (+-camphorate), camsylate (camsilate, camphorsulfonic acid), carbonate, cholate, chloride, chlorogenate, chlorophenoxyacetate, cis-cinnamate, trans-cinnamate, citrate, closylate (4-chlorobenzene sulfonate), cromesilate (6,7-Dihydroxycourmarin-4-methanesulfonate), cyclamate, cyclohexanecarboxylate, decanoate (caprate), dehydrocholate, 2,5-dihydroxybenzoate (gentisate), edetate (EDTA), edisylate (1,2-ethanedisulfonate), erythorbate (isoascorbate), estolate (laurylsulfate), esylate (ethanesulfonate), ethylsulfate, fendizoate, ferulate, formate, fumarate, gluceptate, glucoheptanoate (2,3,4,5,6,7-hexahydroxyheptanoic acid), gluconate (D-gluconate), glucuronate, glutamate, glutarate, glycerophosphate, glycinate, glycolate, glycyrrhizate, glyoxylate (oxaldehydate), hexanoate, hippurate, hydrobromide, hydrochloride, hydroiodide, 4-hydroxybenzenesulfonate, 3-hydroxybenzoate, 4-hydroxybenzoate, hydroxynaphthoate, 3-hydroxybutanoate, 4-hydroxybutanoate, 4-hydroxycinnamate (p-coumarate), 3-hydroxypropionate, iodide, isethionate (2-hydroxyethanesulfonate), isopropyl mesylate (isopropyl methanesulfonate), lactate (D,L-lactate), lactobionate (erythromycin mono (4-0-β-D-galactopyranosyl-D-gluconate)), laurate, lipoate ((R)-lipoate), lysine, malate (2-hydroxybutanedioic acid), maleate ((Z)-but-2-enedioate), mandelate (2-hydroxy-2-phenylacetate), mesotartarate, mesylate (methanesulfonate), metaphosphate, 4-methylpentanoate, 2-methylpropionate, methylsuccinate, methylsulfate, mucate (galactarate; (2S,3R,4S,5R)-2,3,4,5-tetrahydroxyhexanedioate), napadisylate (1,5-naphthalenedisulfonate), napsylate (naphthalene-2-sulfonate), nicotinate, nitrate, octanoate (caprylate), oleate, orotate, oxalate (ethanedioate), oxoglurate, palmitate, pamoate (3-carboxy-1-[(3-carboxy-2-oxidonaphthalen-1-yl)methyl]naphthalen-2-olate), pantothenate (Vitamin B5), pectinate, pentanoate, phenylacetate, phenylethylbarbiturate, phosphate, picrate, pimelate (heptanedioate), polygalacturonate (pectate), polyglutamate, propionate (propanoate), pyridoxal phosphate, L-pyroglutamate, pyruvate, quinate, saccharate, saccharinate, salicylate, sebacate, sinapate, sorbate, stearate, stearylsulfate, succinate, sulfate, sulfosalicylate, tannate, tartrate, teoclate, terephthalate, thiocyanate, tosylate (toluene 4-sulfonate), trans-coutarate, trimethylacetate, undecanoate (undecylate), urate and xinafoate (1-hydroxy-2-naphthoate).

In one aspect of this embodiment, the amount of methyl-β-cyclodextrin in the aqueous formulation can range from about 10% to about 90%, particularly about 50 to about 70% based on the weight of the aqueous formulation. In another aspect of this embodiment, the amount of methyl-p-cyclodextrin in the aqueous formulation can be greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, or greater than about 90%.

In one aspect of this embodiment, the concentration of acceptable salt of tofacitinib or tofacitinib free base can be at least about 20 mg/mL, at least about 30 mg/mL, at least about 40 mg/mL, at least about 50 mg/mL, at least about 60 mg/mL, at least about 70 mg/mL, at least about 80 mg/mL, at least about 90 mg/mL, at least about 100 mg/mL, at least about 110 mg/mL, at least about 120 mg/mL, at least about 130 mg/mL, at least about 140 mg/mL, at least about 150 mg/mL, at least about 160 mg/mL, at least about 170 mg/mL, at least about 180 mg/mL, at least about 190 mg/mL, or at least about 200 mg/mL. In another aspect of this embodiment, the tofacitinib salt can be tofacitinib citrate, and the tofacitinib citrate concentration can be at least about 20 mg/mL, at least about 30 mg/mL, at least about 40 mg/mL, at least about 50 mg/mL, at least about 60 mg/mL, at least about 70 mg/mL, at least about 80 mg/mL, at least about 90 mg/mL, at least about 100 mg/mL, at least about 110 mg/mL, at least about 120 mg/mL, at least about 130 mg/mL, at least about 140 mg/mL, at least about 150 mg/mL, at least about 160 mg/mL, at least about 170 mg/mL, at least about 180 mg/mL, at least about 190 mg/mL, or at least about 200 mg/mL.

In one aspect of this embodiment, the aqueous formulation can further comprise a stabilizer. In a specific aspect of this embodiment, the stabilizer can be a polymer. In another specific aspect of this embodiment, the polymer can be a polyacrylic acid polymer. In some aspects, the polyacrylic acid polymer can be a crosslinked polyacrylic acid polymer. In some aspects, the polyacrylic acid polymer can be a homopolymer of acrylic acid crosslinked with allyl sucrose or allyl pentaerythritol. In yet another specific aspect, the crosslinked polyacrylic acid polymer can be a Carbomer. In still another specific aspect, the Carbomer may be selected from the group consisting of Carbomer homopolymer type A, Carbomer homopolymer type B, Carbomer homopolymer type C, Carbomer copolymer, Carbomer interpolymer, or a combination thereof. In some other aspects, the polyacrylic acid polymer can be a Carbopol® copolymer. In some further aspects, the Carbopol® copolymer can be a copolymer of an acrylic acid and a C₁₀-C₃₀ alkyl acrylate crosslinked with allyl pentaerythritol. In some other aspects, the polyacrylic acid polymer can be a Carbopol® interpolymer. In some further aspects, the Carbopol® interpolymer can be a Carbopol homopolymer or copolymer containing a block copolymer of polyethylene glycol and a long chain alkyl acid ester. In a specific aspect of this embodiment, the stabilizer can be selected from a group consisting of Carbopol® 71G, Carbopol® 971P, Carbopol® 974P, Carbopol® 980, Carbopol® 981, Carbopol® 5984, Carbopol® 934, Carbopol® 934P, Carbopol® 940; Carbopol® 941; Carbopol 950, Carbopol 980, Carbopol 951 and Carbopol 981, Carbopol® 5984, and a combination of any two or more of the foregoing.

In one aspect of its embodiment, the concentration of the stabilizer can range from about 0.05% w/w to about 3.0 w/w, from about 0.1% w/w to about 2.5% w/w, from about 0.25% w/w to about 2.0% w/w, or from about 0.5% w/w to about 1.5% w/w.

In one aspect of this embodiment, the pH of the aqueous formulation can range from about 3.6 to about 8.0. In another aspect of this embodiment, the pH of the aqueous formulation can be about 3.6, about 3.7, about 3.8, about 3.9, about 4, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0. In a specific exemplary embodiment, the pH of the aqueous formulation can be in the range of about 4.5 to about 7.5, about 5 to about 8, or about 5 to about 7.

In one exemplary embodiment, the aqueous formulation can comprise a pharmaceutically acceptable salt of tofacitinib or tofacitinib free base and sulfobutylether-β-cyclodextrin. In one aspect of this embodiment, the tofacitinib salt form can be an acid addition salt. In another aspect of this embodiment, the tofacitinib salt can be selected from the group consisting of tofacitinib aceglutamate, acephyllinate, acetamidobenzoate, 2-acetamidobenzoate, 4-acetamidobenzoate, acetate, acetylasparaginate, acetylaspartate, acid citrate, adipate, acetylsalicylate, aminosalicylate, anhydromethylenecitrate, ascorbate, aspartate, benzenesulfonate (besylate), benzoate, bicarbonate, bisulfate, bitartrate, borate, bromide, butanoate, caffeate, caftarate, camphorate (+-camphorate), camsylate (camsilate, camphorsulfonic acid), carbonate, cholate, chloride, chlorogenate, chlorophenoxyacetate, cis-cinnamate, trans-cinnamate, citrate, closylate (4-chlorobenzene sulfonate), cromesilate (6,7-Dihydroxycourmarin-4-methanesulfonate), cyclamate, cyclohexanecarboxylate, decanoate (caprate), dehydrocholate, 2,5-dihydroxybenzoate (gentisate), edetate (EDTA), edisylate (1,2-ethanedisulfonate), erythorbate (isoascorbate), estolate (laurylsulfate), esylate (ethanesulfonate), ethylsulfate, fendizoate, ferulate, formate, fumarate, gluceptate, glucoheptanoate (2,3,4,5,6,7-hexahydroxyheptanoic acid), gluconate (D-gluconate), glucuronate, glutamate, glutarate, glycerophosphate, glycinate, glycolate, glycyrrhizate, glyoxylate (oxaldehydate), hexanoate, hippurate, hydrobromide, hydrochloride, hydroiodide, 4-hydroxybenzenesulfonate, 3-hydroxybenzoate, 4-hydroxybenzoate, hydroxynaphthoate, 3-hydroxybutanoate, 4-hydroxybutanoate, 4-hydroxycinnamate (p-coumarate), 3-hydroxypropionate, iodide, isethionate (2-hydroxyethanesulfonate), isopropyl mesylate (isopropyl methanesulfonate), lactate (D,L-lactate), lactobionate (erythromycin mono (4-0-β-D-galactopyranosyl-D-gluconate)), laurate, lipoate ((R)-lipoate), lysine, malate (2-hydroxybutanedioic acid), maleate ((Z)-but-2-enedioate), mandelate (2-hydroxy-2-phenylacetate), mesotartarate, mesylate (methanesulfonate), metaphosphate, 4-methylpentanoate, 2-methylpropionate, methylsuccinate, methylsulfate, mucate (galactarate; (2S,3R,4S,5R)-2,3,4,5-tetrahydroxyhexanedioate), napadisylate (1,5-naphthalenedisulfonate), napsylate (naphthalene-2-sulfonate), nicotinate, nitrate, octanoate (caprylate), oleate, orotate, oxalate (ethanedioate), oxoglurate, palmitate, pamoate (3-carboxy-1-[(3-carboxy-2-oxidonaphthalen-1-yl)methyl]naphthalen-2-olate), pantothenate (Vitamin B5), pectinate, pentanoate, phenylacetate, phenylethylbarbiturate, phosphate, picrate, pimelate (heptanedioate), polygalacturonate (pectate), polyglutamate, propionate (propanoate), pyridoxal phosphate, L-pyroglutamate, pyruvate, quinate, saccharate, saccharinate, salicylate, sebacate, sinapate, sorbate, stearate, stearylsulfate, succinate, sulfate, sulfosalicylate, tannate, tartrate, teoclate, terephthalate, thiocyanate, tosylate (toluene 4-sulfonate), trans-coutarate, trimethylacetate, undecanoate (undecylate), urate and xinafoate (1-hydroxy-2-naphthoate).

In one aspect of this embodiment, the amount of sulfobutylether-β-cyclodextrin in the aqueous formulation can range from about 10% to about 90%, particularly about 30% to about 70%, more particularly about 30% to about 50% based on the weight of the aqueous formulation. In another aspect of this embodiment, the amount of sulfobutylether-β-cyclodextrin in the aqueous formulation can be greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, or greater than about 90%.

In one aspect of this embodiment, the concentration of acceptable salt of tofacitinib or tofacitinib free base can be at least about 20 mg/mL, at least about 30 mg/mL, at least about 40 mg/mL, at least about 50 mg/mL, at least about 60 mg/mL, at least about 70 mg/mL, at least about 80 mg/mL, at least about 90 mg/mL, at least about 100 mg/mL, at least about 110 mg/mL, at least about 120 mg/mL, at least about 130 mg/mL, at least about 140 mg/mL, at least about 150 mg/mL, at least about 160 mg/mL, at least about 170 mg/mL, at least about 180 mg/mL, at least about 190 mg/mL, or at least about 200 mg/mL. In another aspect of this embodiment, the tofacitinib salt can be tofacitinib citrate, and the tofacitinib citrate concentration can be at least about 20 mg/mL, at least about 30 mg/mL, at least about 40 mg/mL, at least about 50 mg/mL, at least about 60 mg/mL, at least about 70 mg/mL, at least about 80 mg/mL, at least about 90 mg/mL, at least about 100 mg/mL, at least about 110 mg/mL, at least about 120 mg/mL, at least about 130 mg/mL, at least about 140 mg/mL, at least about 150 mg/mL, at least about 160 mg/mL, at least about 170 mg/mL, at least about 180 mg/mL, at least about 190 mg/mL, or at least about 200 mg/mL.

In one aspect of this embodiment, the aqueous formulation can further comprise a stabilizer. In a specific aspect of this embodiment, the stabilizer can be a polymer. In another specific aspect of this embodiment, the polymer can be a polyacrylic acid polymer. In some aspects, the polyacrylic acid polymer can be a crosslinked polyacrylic acid polymer. In some aspects, the polyacrylic acid polymer can be a homopolymer of acrylic acid crosslinked with allyl sucrose or allyl pentaerythritol. In yet another specific aspect, the crosslinked polyacrylic acid polymer can be a Carbomer. In still another specific aspect, the Carbomer may be selected from the group consisting of Carbomer homopolymer type A, Carbomer homopolymer type B, Carbomer homopolymer type C, Carbomer copolymer, Carbomer interpolymer, or a combination thereof. In some other aspects, the polyacrylic acid polymer can be a Carbopol® copolymer. In some further aspects, the Carbopol® copolymer can be a copolymer of an acrylic acid and a C₁₀-C₃₀ alkyl acrylate crosslinked with allyl pentaerythritol. In some other aspects, the polyacrylic acid polymer can be a Carbopol® interpolymer. In some further aspects, the Carbopol® interpolymer can be a Carbopol homopolymer or copolymer containing a block copolymer of polyethylene glycol and a long chain alkyl acid ester. In a specific aspect of this embodiment, the stabilizer can be selected from a group consisting of Carbopol® 71G, Carbopol® 971P, Carbopol® 974P, Carbopol® 980, Carbopol® 981, Carbopol® 5984, Carbopol® 934, Carbopol® 934P, Carbopol® 940; Carbopol® 941; Carbopol 950, Carbopol 980, Carbopol 951 and Carbopol 981, Carbopol® 5984, and a combination of any two or more of the foregoing.

In one aspect of its embodiment, the concentration of the stabilizer can range from about 0.05% w/w to about 3.0 w/w, from about 0.1% w/w to about 2.5% w/w, from about 0.25% w/w to about 2.0% w/w, or from about 0.5% w/w to about 1.5% w/w.

In one aspect of this embodiment, the pH of the aqueous formulation can range from about 3.6 to about 8.0. In another aspect of this embodiment, the pH of the aqueous formulation can be about 3.6, about 3.7, about 3.8, about 3.9, about 4, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0. In a specific exemplary embodiment, the pH of the aqueous formulation can be in the range of about 4.5 to about 7.5, about 5 to about 8, or about 5 to about 7.

The disclosure, at least in part, provides a method for treating a disease or condition in a subject.

In one exemplary embodiment the method for treating a disease or condition in a subject can comprise administering to a subject in need thereof an aqueous formulation as described in embodiments and aspects described herein.

In one aspect of this embodiment, the disease or condition can be a gastrointestinal disease or condition. In a specific aspect of this embodiment, the disease or condition can be an inflammatory bowel disease. In a specific aspect of this embodiment, the inflammatory bowel disease can be ulcerative colitis. In another specific aspect of this embodiment, the inflammatory bowel disease can be Crohn's disease. In yet another specific aspect of this embodiment, the inflammatory bowel disease can be ileal Crohn's disease.

In one aspect of this embodiment, the method of treating a disease or condition in a subject can comprise administering the aqueous formulation to (a) to a section or subsection of the gastrointestinal (GI) tract of the subject; or (b) proximal to a section or subsection of the gastrointestinal (GI) tract of the subject. In a specific aspect of this embodiment, the section or subsection of the GI tract can contain one or more disease sites. In one aspect of this embodiment, the one or more disease sites can be selected from the group consisting of stomach, duodenum, jejunum, ileum, cecum, ascending colon, transverse colon, descending colon, sigmoid colon and rectum; preferably, the section or subsection of the GI tract containing the one or more disease sites can be selected from the group consisting of ileum, cecum, colon and rectum; and a combination thereof.

In one aspect of this embodiment, the method of treating a disease or condition in a subject can comprise administering the aqueous formulation by an oral route.

In one aspect of this embodiment, the method of treating a disease or condition in a subject can comprise administering the aqueous formulation by a topical route.

In one aspect of this embodiment, the method of treating a disease or condition in a subject can comprise administering the aqueous formulation by a rectal route.

In one aspect of this embodiment, the aqueous formulation can be contained in a device selected from an endoscope, an ingestible device, or a reservoir.

In one aspect of this embodiment, the endoscope can comprise a catheter. In a specific aspect of this embodiment, the catheter can be a spray catheter. In another specific aspect of this embodiment, the endoscope can be connected to the reservoir. In yet another specific aspect of this embodiment, the endoscope can be connected to an anchorable reservoir.

In one aspect of this embodiment, the reservoir can be an anchorable reservoir.

In one aspect of this embodiment, the aqueous formulation can be administered via rectal administration. In another of this embodiment, the aqueous formulation can be an enema for rectal administration. In a specific aspect of this embodiment, the aqueous formulation can be an enema that treats one or more sites of the disease or condition in the sigmoid colon, the rectum, or both.

In one aspect of this embodiment, the aqueous formulation can be administered using an ingestible device.

In a specific aspect of this embodiment, the aqueous formulation can be administered using an ingestible device, said device can comprise a housing, a reservoir containing the aqueous formulation, and a release mechanism for releasing the aqueous formulation from the device, wherein the reservoir can be releasably or permanently attached to the exterior of the housing or internal to the housing. In another specific aspect of this embodiment, the said device can comprise a housing, a reservoir containing the aqueous formulation, and a release mechanism for releasing the aqueous formulation from the device, wherein the reservoir can be internal to the device.

In another specific aspect of this embodiment, the aqueous formulation can be administered using an ingestible device, said device can comprise a self-localization mechanism configured to determine a device location within the subject's GI tract.

In another specific aspect of this embodiment, the aqueous formulation can be administered using an ingestible device, said device can comprise a self-localization mechanism configured to determine a device location within the subject's GI tract and the method can further include determining the device location within the subject's GI tract via a device self-localization mechanism.

In one aspect of this embodiment, determining the device location within the subject's GI tract via a device self-localization mechanism can include detecting one or more device transitions between portions of the subject's GI tract. In a specific aspect of this embodiment, the one or more device transitions occurs between portions of the GI tract selected from the group consisting of: mouth and stomach; esophagus and stomach; stomach and duodenum; duodenum and jejunum; jejunum and ileum; ileum and cecum; and cecum and ascending colon; and combinations of any two or more of the foregoing. In some aspects, the portions are adjacent portions. In another specific aspect of this embodiment, the device self-localization mechanism can be based on data comprising light reflectance occurring external to the device and within the GI tract of the subject. In some aspects, the device self-localization mechanism can be based on data comprising elapsed time after entry of the device into the GI tract of the subject, elapsed time after detecting at least one of the one or more device transitions between the portions of the subject's GI tract, or a combination thereof. In some aspects, determining the device location within the subject's GI tract via the device self-localization mechanism can further include confirming the one or more device transition between the portions of the GI tract of the subject. In some aspects, the device can self-localize to the stomach, duodenum, jejunum, ileum, cecum or colon with at least about 80% accuracy. In some aspects, the device can self-localize to the stomach, duodenum, jejunum, ileum, cecum or colon with at least about 85% accuracy. In some aspects, the self-localization of the device to a pre-selected location within the subject's GI tract autonomously triggers a release of the formulation from the device. In some aspects, the release of the formulation from the device can be proximal to the section or subsection of the subject's GI tract containing at least one of the one or more disease sites. In some aspects, the release of the formulation can be to a section or subsection of the GI tract immediately proximal (immediately preceding) the section or subsection of the subject's GI tract containing at least one of the one or more disease sites.

In another specific aspect of this embodiment, the release of the formulation from the device can occur at substantially the same time as the device self-localizes to the pre-selected location. In some aspects, the release of the formulation from the device commences within a period of time of at most about 5 minutes after the device detects or confirms the transition to the pre-selected location. In some aspects, the period of time can be at most about 1 minute, at most about 30 seconds, at most about 10 seconds, or at most about 1 second after the device detects or confirms the transition to the pre-selected location. In some aspects, the release of the formulation from the device can occur over a pre-determined period of time. In some aspects, the pre-determined period of time can be about 8 hours, about 7 hours, about 6 hours, about 5 hours, about 4 hours, about 3 hours, about 2 hours, about 1 hour, about 30 minutes, about 15 minutes, about 10 minutes, or about 5 minutes. In some aspects, the pre-determined period of time can commence within at most about 5 minutes, at most about 1 minute, at most about 30 seconds, at most about 10 seconds, or at most about 1 second after the device detects or confirms the transition to the pre-selected location.

In one aspect of this embodiment, release of the aqueous formulation from the device can be triggered autonomously.

In one aspect of this embodiment, release of the aqueous formulation from the device can be programmed to release the aqueous formulation at a location proximate to one or more sites of the disease or the condition. In a specific aspect of this embodiment, release of the aqueous formulation can be triggered by a pre-programmed algorithm. In another specific aspect of this embodiment, release of the aqueous formulation can be triggered by data from a sensor or detector to identify the location of the device. In yet another specific aspect of this embodiment, release of the aqueous formulation can be triggered based on a detected reflectance from an external environment.

In one aspect of this embodiment, the device can further comprise one more machine-readable hardware storage devices storing instructions that are executable by the one or more processing devices to (a) determine a location of the ingestible device in the GI tract of the subject; and (b) release the formulation from the device at a pre-selected location of the GI tract. In a specific aspect of this embodiment, the device can further comprise a force generator that generates a force, thereby initiating a release of the formulation from the ingestible device into a pre-selected location of the GI tract. In a further specific aspect of this embodiment, the force generator can be a gas generating cell that generates a gas.

### DETAILED DESCRIPTION

The present disclosure recognizes that, while the commercial immediate release tablet dosage form provides efficacious blood levels of tofacitinib to subjects (dictated by the average blood plasma concentration of tofacitinib, Cₐᵥₑ, over a 24-hour period), it is not an effective way of delivering drug to the site of action. The use of an aqueous formulation delivered locally at the site of disease can enhance response and reduce risks of adverse systemic effects.

The present disclosure also recognizes that a major obstacle in the preparation of a high strength aqueous formulation of tofacitinib and/or its acid addition salts is its pH dependent aqueous solubility. The present disclosure seeks to provide an aqueous formulation and a method of making an aqueous formulation that overcomes this major obstacle by increasing the apparent solubility of tofacitinib and/or its acid addition salts in the formulation matrix. Thus, these aqueous formulations can allow solubilizing a high concentration of an active pharmaceutical ingredient (API) and a delivery of high payload of drug to the gastrointestinal (GI) tract.

The present disclosure further recognizes that oral delivery of an active pharmaceutical ingredient, in the form of an aqueous formulation or otherwise, can also present challenges. The traditional oral delivery of a drug may lend itself to systemic exposure associated with undesirable or potentially harmful side effects. Another challenge associated with oral administration relates to potential instability of the drug upon exposure to the harsh chemical and/or enzymatic degradation conditions of the GI tract. Additionally, for gastrointestinal inflammatory disorders, active pharmaceutical ingredients(s) may need to be dispensed to specified locations within the small intestine or large intestine, which can be more effective than traditional oral administration of the therapeutic drugs to cure or alleviate the symptoms of some medical conditions. For example, therapeutic drugs dispensed directly within the small or large intestine would not be contaminated, digested or otherwise compromised in the stomach, and thus allow a higher dose to be delivered at a specific location within the intestine. Furthermore, therapeutic drugs dispensed directly within the small or large intestine can bypass first pass metabolism and/or small intestinal enzymes and/or problems associated with poor oral bioavailability (e.g., bioavailability requiring uptake in small intestine and entry into systemic circulation before reaching target disease site, which in some cases may be in colon).

The present disclosure further recognizes that aqueous formulations with a high concentration of an active pharmaceutical ingredient (API) or a high payload of drug delivered topically, via administration with an endoscope equipped with a spray catheter, an enema, or via oral administration of an ingestible device configured to dispense the aqueous formulation can overcome the aforementioned challenges.

Finally, the present disclosure recognizes a need for an improved formulation which can provide higher payload of tofacitinib and/or its acid addition salts to the target site in the GI tract and thus provide a markedly higher local concentration of drug in colonic tissue compared to the concentration of drug circulating in plasma, thereby offering the potential to treat inflammatory diseases of the GI tract with reduced risks of side effects associated with systemic exposure. Accordingly, the present disclosure is concerned with and directed to aqueous formulations for aqueous formulations containing active pharmaceutical ingredient and cyclodextrin, to their method of preparation and their uses.

Further aspects, features and advantages of the exemplary embodiments will become apparent from the detailed description which follows.

As used herein, the terms “include,” “includes,” and “including,” are meant to be non-limiting and are understood to mean “comprise,” “comprises,” and “comprising,” respectively.

The term “a” should be understood to mean “at least one”; and the terms “about” and “approximately” should be understood to permit standard variation as would be understood by those of ordinary skill in the art; and where ranges are provided, endpoints are included.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described. All publications mentioned are hereby incorporated by reference.

In the specification and claims, the singular forms include plural referents unless the context clearly dictates otherwise. As used herein, unless specifically indicated otherwise, the word “or” is used in the “inclusive” sense of “and/or” and not the “exclusive” sense of “either/or.”

Technical and scientific terms used herein have the meaning commonly understood by one of skill in the art to which the present description pertains, unless otherwise defined. Reference is made herein to various methodologies and materials known to those of skill in the art. Standard reference works setting forth the general principles of pharmacology and pharmaceutics include LAURENCE L. BRUNTON ET AL., GOODMAN & GILMANS THE PHARMACOLOGICAL BASIS OF THERAPEUTICS (2001) and LOYD V. ALLEN, REMINGTON THE SCIENCE AND PRACTICE OF PHARMACY (2013).

As used herein, whether in a transitional phrase or in the body of a claim, the terms “comprise(s)” and “comprising” are to be interpreted as having an open-ended meaning. That is, the terms are to be interpreted synonymously with the phrases “having at least” or “including at least”. When used in the context of an aqueous formulation, the term “comprising” means that the aqueous formulation includes at least the recited features or components, but may also include additional features or components. When used in the context of a method, the term “comprising” means that the method includes at least the recited steps, but may include additional steps.

The terms “consists essentially of” or “consisting essentially of” have a partially closed meaning, that is, they do not permit inclusion of steps or features or components which would substantially change the essential characteristics of a method or aqueous formulation; for example, steps or features or components which would significantly interfere with the desired properties of the compounds or aqueous formulations described herein, i.e., the aqueous formulation or method can be limited to the specified steps or materials and those which do not materially affect the basic and novel characteristics of the aqueous formulation or method.

The terms “consists of” and “consists” are closed terminology and allow only for the inclusion of the recited components or features or steps.

Poor aqueous solubility and rate of dissolution are the two critical factors that affect the formulation and development process of several active pharmaceutical ingredients and limit their therapeutic application. Although several techniques like solubilization, co-solvency, and solid dispersion can enhance drug's solubility, bioavailability, and dissolution properties, these methods suffer from various disadvantages such as low drug loading and large dose. As an alternative, use of cyclodextrin (CD) has emerged as a novel plan to tackle such formulation problems.

In some exemplary embodiments, the disclosure provides an aqueous formulation comprising an active pharmaceutical ingredient and cyclodextrin.

As used herein, the “active pharmaceutical ingredient” or “API” can be referred to as a “drug” or a “therapeutic agent.”

In some exemplary embodiments, the API can be a JAK inhibitor.

As used herein, “JAK” or “Janus kinase” are proteins which are a family of non-receptor tyrosine kinases that possess a highly conserved kinase domain responsible for its enzymatic activity. Mammals have four members of this family, JAK1, JAK2, JAK3 and Tyrosine kinase 2 (TYK2). These kinases associate with the intracellular portion of cytokine or hormone receptors, and transduce signals through seven members of the STAT transcription factors—STAT1, STAT2, STAT3, STAT4, STAT5A, STAT5B, and STAT6 in various combinations. JAK1 promotes signaling of multiple cytokines (e.g., certain type I and type II cytokines), including pro-inflammatory cytokines involved in the pathogenesis of autoimmune diseases. Single-nucleotide polymorphisms within the JAK/STAT pathway that confer susceptibility to IBD have been identified (Jostins et al., Host-microbe interactions have shaped the genetic architecture of inflammatory bowel disease, Nature 491:119-124, 2012). Both in vivo and in vitro studies have confirmed the role of JAK/STAT signaling in regulating immune responses (O'Shea and Plenge, JAK and STAT signaling molecules in immunoregulation and immune-mediated disease, Immunity 36:542-550, 2012). Representative JAK inhibitors can include those disclosed in U.S. Pat. No. 7,598,257, an example of which can be Ruxolitinib (Jakafi, Incyte). Representative JAK inhibitors can also include those disclosed in U.S. Pat. Nos. Re 41,783; 7,842,699; 7,803,805; 7,687,507; 7,601,727; 7,569,569; 7,192,963; 7,091,208; 6,890,929, 6,696,567; 6,962,993; 6,635,762; 6,627,754; and 6,610,847, an example of which can be tofacitinib (Pfizer). These references are incorporated herein in their entirety.

In some exemplary embodiments, the API can be tofacitinib.

As used herein, “tofacitinib” can also be referred to as 4-methyl-3-[methyl-(7H-pyrrolo[2,3-d]pyrimidin-4-yl)-amino]-piperidin-1-yl}-3-oxo-propionitrile or (3-{(3R,4R)-4-methyl-3-[methyl-(7H-pyrrolo[2,3-d]pyrimidin-4-yl)-amino]-piperidin-1-yl}-3-oxo-propionitrile) or C₁₆H₂₀N₆₀ or CP-690550 or tasocitinib, which has the following structure:

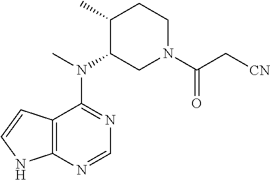 [SMILES: C[C@@H]1CCN(C(=O)CC#N)C[C@@H]1N(C)c1ncnc2[nH]ccc12]

Tofacitinib is an inhibitor of JAK enzymes, including JAK1, JAK2 and JAK3. It has been investigated as an immunosuppressive agent for the therapy of several conditions such as organ transplants, xeno transplantation, lupus, multiple sclerosis, rheumatoid arthritis, psoriasis, Type I diabetes and complications from diabetes, cancer, asthma, atopic dermatitis, autoimmune thyroid disorders, ulcerative colitis, Crohn's disease, Alzheimer's disease, leukemia and other indications, where immunosuppression would be desirable (see WO 03/048126). In the US and Europe, oral dosage forms of tofacitinib citrate (XELJANZ®, JAKVINUS®, tasocitinib citrate, CP-690550 citrate) have been approved for use in the treatment of rheumatoid arthritis, psoriatic arthritis and ulcerative colitis (UC), with some limitations. For example, use of XELJANZ in combination with biologic therapies for UC or with potent immunosuppressants such as azathioprine and cyclosporine can be not recommended. Moreover, patients treated with XELJANZ/XELJANZ XR are at increased risk for developing serious infections that may lead to hospitalization or death. Most patients who developed these infections were taking concomitant immunosuppressants, such as methotrexate or corticosteroids. Lymphoma and other malignancies have been observed in patients treated with XELJANZ. Epstein Barr Virus-associated post-transplant lymphoproliferative disorder has been observed at an increased rate in renal transplant patients treated with XELJANZ and concomitant immunosuppressive medications. As reported by European Medicines Agency, tofacitinib is classified as a Biopharmaceutics Classification System (BCS) Class III compound (e.g., high solubility, low permeability) (Assessment report; Xeljanz; Procedure No. EMEA/H/C/002542/0000; EMA/CHMP/425279/2013: Committee for Medicinal Products for Human Use (CHMP) published on Jul. 25, 2013, page 62/197).

In some other exemplary embodiments, the API can be a pharmaceutically acceptable salt of tofacitinib. Non-limiting examples of acceptable salt form of tofacitinib can include aceglutamate, acephyllinate, acetamidobenzoate, 2-acetamidobenzoate, 4-acetamidobenzoate, acetate, acetylasparaginate, acetylaspartate, acid citrate, adipate, acetylsalicylate, aminosalicylate, anhydromethylenecitrate, ascorbate, aspartate, benzenesulfonate (besylate), benzoate, bicarbonate, bisulfate, bitartrate, borate, bromide, butanoate, caffeate, caftarate, camphorate (+-camphorate), camsylate (camsilate, camphorsulfonic acid), carbonate, cholate, chloride, chlorogenate, chlorophenoxyacetate, cis-cinnamate, trans-cinnamate, citrate, closylate (4-chlorobenzene sulfonate), cromesilate (6,7-Dihydroxycourmarin-4-methanesulfonate), cyclamate, cyclohexanecarboxylate, decanoate (caprate), dehydrocholate, 2,5-dihydroxybenzoate (gentisate), edetate (EDTA), edisylate (1,2-ethanedisulfonate), erythorbate (isoascorbate), estolate (laurylsulfate), esylate (ethanesulfonate), ethylsulfate, fendizoate, ferulate, formate, fumarate, gluceptate, glucoheptanoate (2,3,4,5,6,7-hexahydroxyheptanoic acid), gluconate (D-gluconate), glucuronate, glutamate, glutarate, glycerophosphate, glycinate, glycolate, glycyrrhizate, glyoxylate (oxaldehydate), hexanoate, hippurate, hydrobromide, hydrochloride, hydroiodide, 4-hydroxybenzenesulfonate, 3-hydroxybenzoate, 4-hydroxybenzoate, hydroxynaphthoate, 3-hydroxybutanoate, 4-hydroxybutanoate, 4-hydroxycinnamate (p-coumarate), 3-hydroxypropionate, iodide, isethionate (2-hydroxyethanesulfonate), isopropyl mesylate (isopropyl methanesulfonate), lactate (D,L-lactate), lactobionate (erythromycin mono (4-0-β-D-galactopyranosyl-D-gluconate)), laurate, lipoate ((R)-lipoate), lysine, malate (2-hydroxybutanedioic acid), maleate ((Z)-but-2-enedioate), mandelate (2-hydroxy-2-phenylacetate), mesotartarate, mesylate (methanesulfonate), metaphosphate, 4-methylpentanoate, 2-methylpropionate, methylsuccinate, methylsulfate, mucate (galactarate; (2S,3R,4S,5R)-2,3,4,5-tetrahydroxyhexanedioate), napadisylate (1,5-naphthalenedisulfonate), napsylate (naphthalene-2-sulfonate), nicotinate, nitrate, octanoate (caprylate), oleate, orotate, oxalate (ethanedioate), oxoglurate, palmitate, pamoate (3-carboxy-1-[(3-carboxy-2-oxidonaphthalen-1-yl)methyl]naphthalen-2-olate), pantothenate (Vitamin B5), pectinate, pentanoate, phenylacetate, phenylethylbarbiturate, phosphate, picrate, pimelate (heptanedioate), polygalacturonate (pectate), polyglutamate, propionate (propanoate), pyridoxal phosphate, L-pyroglutamate, pyruvate, quinate, saccharate, saccharinate, salicylate, sebacate, sinapate, sorbate, stearate, stearylsulfate, succinate, sulfate, sulfosalicylate, tannate, tartrate, teoclate, terephthalate, thiocyanate, tosylate (toluene 4-sulfonate), trans-coutarate, trimethylacetate, undecanoate (undecylate), urate, and xinafoate (1-hydroxy-2-naphthoate).

In some exemplary embodiments, the pharmaceutically acceptable salt of tofacitinib form can be an acid addition salt of tofacitinib.

In some exemplary embodiments, the acid addition salt of tofacitinib can comprise at least one carboxylic acid group. Non-limiting examples of such a salt form can include aceglutamate (1 COOH, also terminal amino group), acephyllinate (1 COOH substituent of purine-like cmpd), acetamidobenzoate (1 COOH substituent on phenyl), 2-acetamidobenzoate, 4-acetamidobenzoate, acetate, acetylasparaginate (1 COOH), acetylaspartate (1 COOH), acid citrate, adipate (2 COOH), acetylsalicylate, aminosalicylate (1 COOH, 1 free amino, 1 phenolic OH), anhydromethylenecitrate (2 COOH), aspartate (2 COOH, 1 NH2), benzoate, bicarbonate (HO(CO)O), bitartrate (2 COOH, 2 OH), butanoate, caffeate, caftarate (2 COOH), camphorate (+-camphorate; 2 COOH), carbonate (O(CO)O), chlorogenate (1COOH, 3 OH), cis-cinnamate, trans-cinnamate, citrate (3 COOH, 1 OH), cholate (1 COOH plus 30H on steroid), chlorophenoxyacetate (1 COOH), cyclohexanecarboxylate, decanoate (caprate), dehydrocholate (1 COOH on steroid backbone), 2,5-dihydroxybenzoate (gentisate), edetate (EDTA; 4 COOH, 2 NR3), fendizoate (1 COOH, 1 phenolic OH), ferulate (1 COOH), formate, fumarate (2 COOH), gluceptate (1 COOH, 6 OH), glucoheptanoate (2,3,4,5,6,7-hexahydroxyheptanoic acid), gluconate (D-gluconate; 1 COOH, 5 OH), glucuronate (1 COOH, 4 OH), glutamate (2COOH, 1 NH2), glutarate (2 COOH), glycinate (1 COOH, one free amino group), glycolate (1 COOH, 1 OH), glycyrrhizate (3 COOH, 5 OH, substituted steroid), glyoxylate (oxaldehydate; 2 COOH), hexanoate, hippurate (1 COOH), 3-hydroxybenzoate, 4-hydroxybenzoate, 3-hydroxybutanoate (1 COOH, 1 OH), 4-hydroxybutanoate (1 COOH, 1 OH), 4-hydroxycinnamate (p-coumarate) (1 COOH, 1 phenolic OH), 3-hydroxypropionate (1 COOH, 1 OH), hydroxynaphthoate (1 COOH, 1 phenolic OH), lactate (1 COOH, 1 OH), lactobionate (erythromycin mono (4-0-β-D-galactopyranosyl-D-gluconate; 1 COOH, many OH groups)), laurate (1 COOH), lipoate ((R)-lipoate; 1 COOH), lysine, malate (2-hydroxybutanedioic acid; 2 COOH, 1 OH); maleate ((Z)-but-2-enedioate; 2 COOH), mandelate (2-hydroxy-2-phenylacetate), mesotartarate (2 COOH, 2 OH), 4-methylpentanoate, 2-methylpropionate, methylsuccinate (2 COOH), mucate (galactarate; (2S,3R,4S,5R)-2,3,4,5-tetrahydroxyhexanedioate; 2 COOH, 4 OH), nicotinate (1 COOH), octanoate (caprylate; 1 COOH), oleate, orotate (1 COOH), oxalate (ethanedioate; 2 COOH), oxoglurate (1 COOH), palmitate, pamoate (3-carboxy-1-[(3-carboxy-2-oxidonaphthalen-1-yl)methyl]naphthalen-2-olate; 2 COOH, 2 phenolic OH), pantothenate (Vitamin B5; 1 COOH, 2 OH), pectinate, pentanoate, phenylacetate, pimelate (heptanedioate; 2 COOH), polygalacturonate (pectate; 3 COOH, 8 OH), polyglutamate, propionate (propanoate), and L-pyroglutamate, pyruvate.

In some other exemplary embodiments, the pharmaceutically acceptable salt of tofacitinib form can be a halide. The halide can include fluoride, chloride, bromide, or iodide.

In some other exemplary embodiments, the pharmaceutically acceptable salt of tofacitinib form can include butylbromide, dihydrochloride, dimalonate, ethylbromide, methylbromide, methyiodide, methylnitrate, methylsulphate, and triethyliodide.

In a specific exemplary embodiment, the API can be tofacitinib citrate. In an especially preferred exemplary embodiment, the API can be tofacitinib mono-citrate.

Tofacitinib, certain pharmaceutically acceptable salts thereof and their method of preparation, are described in WO 2018/172821, WO2001/042246, WO2002/096909, WO2003/048162, WO2012/135338, WO 2012/135338, WO 2012/137111, WO2013/090490 and U.S. Pat. No. 9,260,438 describes crystalline forms and describes process for preparing certain other tofacitinib salts. These references are incorporated herein in their entirety.

In some exemplary embodiments, the aqueous formulation can comprise the API at a concentration of about 20 mg/mL to about 200 mg/mL. In a specific exemplary embodiment, the aqueous formulation can comprise the API at a concentration of at least about 20 mg/mL, at least about 21 mg/mL, at least about 22 mg/mL, at least about 23 mg/mL, at least about 24 mg/mL, at least about 25 mg/mL, at least about 26 mg/mL, at least about 27 mg/mL, at least about 28 mg/mL, at least about 29 mg/mL, at least about 30 mg/mL, at least about 31 mg/mL, at least about 32 mg/mL, at least about 33 mg/mL, at least about 34 mg/mL, at least about 35 mg/mL, at least about 36 mg/mL, at least about 37 mg/mL, at least about 38 mg/mL, at least about 39 mg/mL, at least about 40 mg/mL, at least about 41 mg/mL, at least about 42 mg/mL, at least about 43 mg/mL, at least about 44 mg/mL, at least about 45 mg/mL, at least about 46 mg/mL, at least about 47 mg/mL, at least about 48 mg/mL, at least about 49 mg/mL, at least about 50 mg/mL, at least about 51 mg/mL, at least about 52 mg/mL, at least about 53 mg/mL, at least about 54 mg/mL, at least about 55 mg/mL, at least about 56 mg/mL, at least about 57 mg/mL, at least about 58 mg/mL, at least about 59 mg/mL, at least about 60 mg/mL, at least about 61 mg/mL, at least about 62 mg/mL, at least about 63 mg/mL, at least about 64 mg/mL, at least about 65 mg/mL, at least about 66 mg/mL, at least about 67 mg/mL, at least about 68 mg/mL, at least about 69 mg/mL, at least about 70 mg/mL, at least about 71 mg/mL, at least about 72 mg/mL, at least about 73 mg/mL, at least about 74 mg/mL, at least about 75 mg/mL, at least about 76 mg/mL, at least about 77 mg/mL, at least about 78 mg/mL, at least about 79 mg/mL, at least about 80 mg/mL, at least about 81 mg/mL, at least about 82 mg/mL, at least about 83 mg/mL, at least about 84 mg/mL, at least about 85 mg/mL, at least about 86 mg/mL, at least about 87 mg/mL, at least about 88 mg/mL, at least about 89 mg/mL, at least about 90 mg/mL, at least about 91 mg/mL, at least about 92 mg/mL, at least about 93 mg/mL, at least about 94 mg/mL, at least about 95 mg/mL, at least about 96 mg/mL, at least about 97 mg/mL, at least about 98 mg/mL, at least about 99 mg/mL, at least about 100 mg/mL, at least about 101 mg/mL, at least about 102 mg/mL, at least about 103 mg/mL, at least about 104 mg/mL, at least about 105 mg/mL, at least about 106 mg/mL, at least about 107 mg/mL, at least about 108 mg/mL, at least about 109 mg/mL, at least about 110 mg/mL, at least about 111 mg/mL, at least about 112 mg/mL, at least about 113 mg/mL, at least about 114 mg/mL, at least about 115 mg/mL, at least about 116 mg/mL, at least about 117 mg/mL, at least about 118 mg/mL, at least about 119 mg/mL, at least about 120 mg/mL, at least about 121 mg/mL, at least about 122 mg/mL, at least about 123 mg/mL, at least about 124 mg/mL, at least about 125 mg/mL, at least about 126 mg/mL, at least about 127 mg/mL, at least about 128 mg/mL, at least about 129 mg/mL, at least about 130 mg/mL, at least about 131 mg/mL, at least about 132 mg/mL, at least about 133 mg/mL, at least about 134 mg/mL, at least about 135 mg/mL, at least about 136 mg/mL, at least about 137 mg/mL, at least about 138 mg/mL, at least about 139 mg/mL, at least about 140 mg/mL, at least about 141 mg/mL, at least about 142 mg/mL, at least about 143 mg/mL, at least about 144 mg/mL, at least about 145 mg/mL, at least about 146 mg/mL, at least about 147 mg/mL, at least about 148 mg/mL, at least about 149 mg/mL, at least about 150 mg/mL, at least about 151 mg/mL, at least about 152 mg/mL, at least about 153 mg/mL, at least about 154 mg/mL, at least about 155 mg/mL, at least about 156 mg/mL, at least about 157 mg/mL, at least about 158 mg/mL, at least about 159 mg/mL, at least about 160 mg/mL, at least about 161 mg/mL, at least about 162 mg/mL, at least about 163 mg/mL, at least about 164 mg/mL, at least about 165 mg/mL, at least about 166 mg/mL, at least about 167 mg/mL, at least about 168 mg/mL, at least about 169 mg/mL, at least about 170 mg/mL, at least about 171 mg/mL, at least about 172 mg/mL, at least about 173 mg/mL, at least about 174 mg/mL, at least about 175 mg/mL, at least about 176 mg/mL, at least about 177 mg/mL, at least about 178 mg/mL, at least about 179 mg/mL, at least about 180 mg/mL, at least about 181 mg/mL, at least about 182 mg/mL, at least about 183 mg/mL, at least about 184 mg/mL, at least about 185 mg/mL, at least about 186 mg/mL, at least about 187 mg/mL, at least about 188 mg/mL, at least about 189 mg/mL, at least about 190 mg/mL, at least about 191 mg/mL, at least about 192 mg/mL, at least about 193 mg/mL, at least about 194 mg/mL, at least about 195 mg/mL, at least about 196 mg/mL, at least about 197 mg/mL, at least about 198 mg/mL, at least about 199 mg/mL, or at least about 200 mg/mL.

In some exemplary embodiments, the aqueous formulation can comprise tofacitinib citrate at a concentration of about 20 mg/mL to about 140 mg/mL. In a specific exemplary embodiment, the aqueous formulation can comprise tofacitinib citrate at a concentration of at least about 20 mg/mL, at least about 21 mg/mL, at least about 22 mg/mL, at least about 23 mg/mL, at least about 24 mg/mL, at least about 25 mg/mL, at least about 26 mg/mL, at least about 27 mg/mL, at least about 28 mg/mL, at least about 29 mg/mL, at least about 30 mg/mL, at least about 31 mg/mL, at least about 32 mg/mL, at least about 33 mg/mL, at least about 34 mg/mL, at least about 35 mg/mL, at least about 36 mg/mL, at least about 37 mg/mL, at least about 38 mg/mL, at least about 39 mg/mL, at least about 40 mg/mL, at least about 41 mg/mL, at least about 42 mg/mL, at least about 43 mg/mL, at least about 44 mg/mL, at least about 45 mg/mL, at least about 46 mg/mL, at least about 47 mg/mL, at least about 48 mg/mL, at least about 49 mg/mL, at least about 50 mg/mL, at least about 51 mg/mL, at least about 52 mg/mL, at least about 53 mg/mL, at least about 54 mg/mL, at least about 55 mg/mL, at least about 56 mg/mL, at least about 57 mg/mL, at least about 58 mg/mL, at least about 59 mg/mL, at least about 60 mg/mL, at least about 61 mg/mL, at least about 62 mg/mL, at least about 63 mg/mL, at least about 64 mg/mL, at least about 65 mg/mL, at least about 66 mg/mL, at least about 67 mg/mL, at least about 68 mg/mL, at least about 69 mg/mL, at least about 70 mg/mL, at least about 71 mg/mL, at least about 72 mg/mL, at least about 73 mg/mL, at least about 74 mg/mL, at least about 75 mg/mL, at least about 76 mg/mL, at least about 77 mg/mL, at least about 78 mg/mL, at least about 79 mg/mL, at least about 80 mg/mL, at least about 81 mg/mL, at least about 82 mg/mL, at least about 83 mg/mL, at least about 84 mg/mL, at least about 85 mg/mL, at least about 86 mg/mL, at least about 87 mg/mL, at least about 88 mg/mL, at least about 89 mg/mL, at least about 90 mg/mL, at least about 91 mg/mL, at least about 92 mg/mL, at least about 93 mg/mL, at least about 94 mg/mL, at least about 95 mg/mL, at least about 96 mg/mL, at least about 97 mg/mL, at least about 98 mg/mL, at least about 99 mg/mL, at least about 100 mg/mL, at least about 101 mg/mL, at least about 102 mg/mL, at least about 103 mg/mL, at least about 104 mg/mL, at least about 105 mg/mL, at least about 106 mg/mL, at least about 107 mg/mL, at least about 108 mg/mL, at least about 109 mg/mL, at least about 110 mg/mL, at least about 111 mg/mL, at least about 112 mg/mL, at least about 113 mg/mL, at least about 114 mg/mL, at least about 115 mg/mL, at least about 116 mg/mL, at least about 117 mg/mL, at least about 118 mg/mL, at least about 119 mg/mL, at least about 120 mg/mL, at least about 121 mg/mL, at least about 122 mg/mL, at least about 123 mg/mL, at least about 124 mg/mL, at least about 125 mg/mL, at least about 126 mg/mL, at least about 127 mg/mL, at least about 128 mg/mL, at least about 129 mg/mL, at least about 130 mg/mL, at least about 131 mg/mL, at least about 132 mg/mL, at least about 133 mg/mL, at least about 134 mg/mL, at least about 135 mg/mL, at least about 136 mg/mL, at least about 137 mg/mL, at least about 138 mg/mL, at least about 139 mg/mL, or at least about 140 mg/mL.

As used herein, the “cyclodextrin” or “CD” can be cyclic oligosaccharides, formed by α-1,4-linked glucose units, with a hydrophilic outer surface and a lipophilic central cavity. In some embodiments, the cyclodextrin has a bucket-like structure with a hydrophobic internal cavity and hydrophilic exterior, which can encapsulate molecules, thereby forming inclusion complexes where lipophilic compounds are non-covalently bound within the cavity.

Cyclodextrins can also be known as cycloamyloses, and can be produced from the enzymatic conversion of starch. They can have a cyclic structure that can be hydrophobic on the inside and hydrophilic on the outside. Because of the amphiphilic nature of the ring, cyclodextrins have been known to enhance the solubility and bioavailability of hydrophobic compounds.

Cyclodextrins can be cyclic oligosaccharides containing 6 (α-cyclodextrin), 7 (β-cyclodextrin), and 8 (γ-cyclodextrin) glucopyranose monomers linked via α-1,4-glycoside bonds. α-Cyclodextrin, 3-cyclodextrin and γ-cyclodextrin are natural products formed by microbial degradation of starch. The outer surface of the doughnut shaped cyclodextrin molecules can be hydrophilic, bearing numerous hydroxyl groups, but their central cavity can be somewhat lipophilic (S V Kurkov & T Loftsson, Cyclodextrins., 453 INTERNATIONAL JOURNAL OF PHARMACEUTICS 167-180 (2013); Thorsteinn Loftsson & Marcus E. Brewster, Pharmaceutical Applications of Cyclodextrins. 1. Drug Solubilization and Stabilization, 85 JOURNAL OF PHARMACEUTICAL SCIENCES 1017-1025 (1996)). In addition to the three natural cyclodextrins, numerous water-soluble cyclodextrin derivatives have been synthesized and tested as drug carriers, including cyclodextrin polymers (V J Stella & Q He, Cyclodextrins., 36 TOXICOLOGIC PATHOLOGY 30-42 (2008)).

Depending on the determination method and the location of the hydroxyl groups, the pKa values of the natural CDs have been reported to be between 12.1 and 13.5. The main difference of the three natural CDs (α-Cyclodextrin, β-cyclodextrin and γ-cyclodextrin), besides the size of their central cavity, can be their aqueous solubility (Phennapha Saokham et al., Solubility of Cyclodextrins and Drug/Cyclodextrin Complexes, 23 MOLECULES 1161 (2018)). The type of CD and substituent can influence the different physicochemical properties (e.g., inner cavity diameter, hydrogen bond acceptors and donors, solubility in water, and effect on surface tension) (Saokham et al, supra).

Highly water-soluble cyclodextrins can be preferred to be used in the present invention, such as, α-cyclodextrin and/or derivatives thereof, γ-cyclodextrin and/or derivatives thereof, derivatized β-cyclodextrins, and/or mixtures thereof. In some exemplary embodiments, highly water-soluble cyclodextrins can have a water solubility of at least about 10 mg/mL of water at room temperature, preferably at least about 20 mg/mL of water, more preferably at least about 25 mg/mL of water at room temperature. The derivatives of cyclodextrin consist mainly of molecules wherein some of the OH groups are converted to OR groups. Cyclodextrin derivatives include, e.g., those with short chain alkyl groups such as methylated cyclodextrins, and ethylated cyclodextrins, wherein R can be a methyl or an ethyl group; those with hydroxyalkyl groups, such as hydroxypropyl cyclodextrins and/or hydroxyethyl cyclodextrins, wherein R can be a —CHCH(OH)—CH or a —CHCH—OH group; those with (hydroxyalkyl)alkylenyl bridging groups such as cyclodextrin glycerol ethers wherein (2-hydroxyethyl)ethylenyl, —CH—CH(CHOH), groups bridge between the 2′ and 3′ hydroxyl oxygens on the glucosyl units, branched cyclodextrins such as maltose-bonded cyclodextrins, cationic cyclodextrins such as those containing 2-hydroxy-3-(dimethylamino)propyl ether, wherein R can be CH—CH(OH)—CH—N(CH) which can be cationic at low pH; quaternary ammonium, e.g., 2-hydroxy-3-(trimethylammonio)propyl ether chloride groups, wherein R can be CH—CH(OH)—CH—N″(CH) Cl; anionic cyclodextrins such as carboxymethyl cyclodextrins, cyclodextrin sulfobutylethers, cyclodextrin sulfates, and cyclodextrin succinylates; amphoteric cyclodextrins such as carboxymethyl/quaternary ammonium cyclodextrins, cyclodextrins wherein at least one glucopyranose unit has a 3-6-anhydrocyclomalto structure, e.g., the mono-3-6-anhydrocyclodextrins, as disclosed in F Diedaini-Pilard & B Perly, Optimal Performances with Minimal Chemical Modification of Cyclodextrins, THE 7TH INTERNATIONAL CYCLODEXTRIN SYMPOSIUM ABSTRACTS 49 (1994), herein incorporated by reference; and mixtures thereof. Other cyclodextrin derivatives are disclosed in U.S. Pat. No. 3,426,011, Parmerter et al., issued Feb. 4, 1969; U.S. Pat. Nos. 3,453,257; 3,453,258; 3,453,259; and 3,453,260, all in the names of Parmerter et al., and all issued Jul. 1, 1969; U.S. Pat. No. 3,459,731, Gramera et al., issued Aug. 5, 1969; U.S. Pat. No. 3,553,191, Parmerter et al., issued Jan. 5, 1971; U.S. Pat. No. 3,565,887, Parmerter et al., issued Feb. 23, 1971; U.S. Pat. No. 4,535,152, Szeitli et al., issued Aug. 13, 1985; U.S. Pat. No. 4,616,008, Hirai et al., issued Oct. 7, 1986; U.S. Pat. No. 4,678,598, Ogino et al., issued Jul. 7, 1987; U.S. Pat. No. 4,638,058, Brandt et al., issued Jan. 20, 1987; and U.S. Pat. No. 4,746,734, Tsuchiyama et al., issued May 24, 1988; U.S. Pat. No. 5,534,165, Pilosof et al., issued Jul. 9, 1996, all of said patents being incorporated herein by reference.

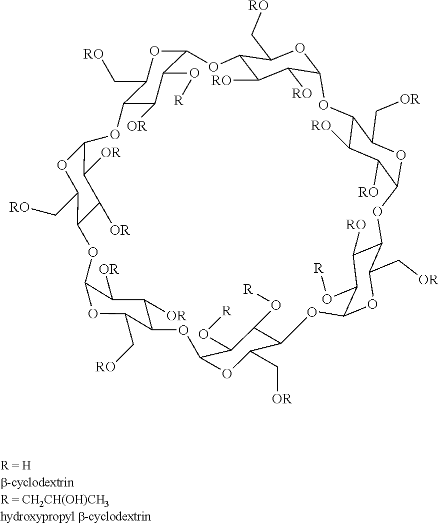

As described herein, cyclodextrins enhances the solubility and bioavailability tofacitinib. Without wishing to be bound by any particular theory, it is contemplated that, in aqueous solutions, cyclodextrins can form inclusion complexes or micelles with tofacitinib by taking up a drug molecule, or more frequently some lipophilic moiety of the molecule, into the central cavity.

Through formulation and stability studies, the Applicants have surprisingly discovered that use of CD in pharmaceutical aqueous formulations with tofacitinib caused a significant increase in the solubility of tofacitinib and as a result aqueous formulations containing much higher concentrations of tofacitinib can be achieved, an insight heretofore unknown.

In some exemplary embodiments, the cyclodextrin can be a natural CD, modified CD or a branched CD.

Non-limiting examples of natural CD can include α-CD, f-CD, or γ-CD.

Non-limiting examples of modified CD can include 2-hydroxypropyl-α-CD, 2-hydroxypropyl-β-CD, 2-hydroxypropyl-γ-CD, sulfobutylether β-CD, sulfobutylether γ-cyclodextrin, dimethyl-α-CD, trimethyl-αCD, methyl-β-CD, carboxymethyl-β-CD, dimethyl-β-CD; trimethyl-β-CD; hydroxyethyl-βCD, dimethyl-γ-CD, trimethyl-γ-CD, hydroxypropyl-γ-CD, sulfobutylether-γCD sodium salt, or sugammadex.

Non-limiting examples of branched CD can include glucosyl-βCD, maltosyl-βCD, and glucoronyl-glucosyl-βCD.

In one specific exemplary embodiment, the cyclodextrin can be hydroxypropyl-β-CD.

In one specific exemplary embodiment, the cyclodextrin can be sulfobutylether β-CD.

In one specific exemplary embodiment, the cyclodextrin can be methyl-CD. In one particular embodiment, the methylcyclodextrin can be a methyl-β-cyclodextrin. In another particular embodiment, the methyl cyclodextrin can be substituted on the hydroxyl borne by the C₂ carbon of the glucopyranose units, or by the C₃ and/or C₆ carbons of the glucopyranose units or by a combination of the C₂, C₃ and/or C₆, preferably C₂ and C₆, carbons of the glucopyranose units.

Non-limiting examples of methyl cyclodextrin are described in WO 2015/087016, U.S. Pat. Nos. 7,259,153, 6,602,860, 5,935,941, 10,022,392 and U.S. Pat. Publication No. 2018/0319903, which are incorporated by reference in their entirety.

The methyl-CD used in the aqueous formulations of the invention preferentially have a have a degree of molecular substitution from about 0 to about 21. As used herein, the “degree of molecular substitution (DS)” can correspond to the number of hydroxyl groups substituted by a methyl group per cyclodextrin molecule and which can therefore take into account the number of glucopyranose units constituting the methylcyclodextrin. DS can have an influence on the physicochemical properties of CDs and their ability to form complexes. Non-limiting examples of methyl-CD include methylated β-CD (CRYSMEB; DC=4; Roquette, France), random methylated β-CD (RAMEB; DC=12; Wacker Chemie, Germany), Heptakis(2,6-di-O-methyl) β-CD (DIMEB; DC=14; CycloLab, Hunagry), or Heptakis(2,3,6-tri-O-methyl) β-CD (TRIMEB; DC=14; CycloLab, Hunagry).

The amount of cyclodextrin in the pharmaceutical aqueous formulation of the disclosure may be from about 10% to about 90%, more particularly about 40 to about 80%, or even more particularly about 50% to about 60% based on the weight of the aqueous formulation.

In some exemplary embodiments, the concentration of CD as the mass percentage of the solute in solution (w/w) can be at least about 1% w/w, at least about 2% w/w, at least about 3% w/w, at least about 4% w/w, at least about 5% w/w, at least about 6% w/w, at least about 7% w/w, at least about 8% w/w, at least about 9% w/w, at least about 10% w/w, at least about 11% w/w, at least about 12% w/w, at least about 13% w/w, at least about 14% w/w, at least about 15% w/w, at least about 16% w/w, at least about 17% w/w, at least about 18% w/w, at least about 19% w/w, at least about 20% w/w, at least about 21% w/w, at least about 22% w/w, at least about 23% w/w, at least about 24% w/w, at least about 25% w/w, at least about 26% w/w, at least about 27% w/w, at least about 28% w/w, at least about 29% w/w, at least about 30% w/w, at least about 31% w/w, at least about 32% w/w, at least about 33% w/w, at least about 34% w/w, at least about 35% w/w, at least about 36% w/w, at least about 37% w/w, at least about 38% w/w, at least about 39% w/w, at least about 40% w/w, at least about 41% w/w, at least about 42% w/w, at least about 43% w/w, at least about 44% w/w, at least about 45% w/w, at least about 46% w/w, at least about 47% w/w, at least about 48% w/w, at least about 49% w/w, at least about 50% w/w, at least about 51% w/w, at least about 52% w/w, at least about 53% w/w, at least about 54% w/w, at least about 55% w/w, at least about 56% w/w, at least about 57% w/w, at least about 58% w/w, at least about 59% w/w, at least about 60% w/w, at least about 61% w/w, at least about 62% w/w, at least about 63% w/w, at least about 64% w/w, at least about 65% w/w, at least about 66% w/w, at least about 67% w/w, at least about 68% w/w, at least about 69% w/w, at least about 70% w/w, at least about 71% w/w, at least about 72% w/w, at least about 73% w/w, at least about 74% w/w, at least about 75% w/w, at least about 76% w/w, at least about 77% w/w, at least about 78% w/w, at least about 79% w/w, at least about 80% w/w, at least about 81% w/w, at least about 82% w/w, at least about 83% w/w, at least about 84% w/w, at least about 85% w/w, at least about 86% w/w, at least about 87% w/w, at least about 88% w/w, at least about 89% w/w, or at least about 90% w/w. In some exemplary embodiments, the concentration of CD as the percent concentration of a material in solution (w/v) can be at least about 1% w/v, at least about 2% w/v, at least about 3% w/v, at least about 4% w/v, at least about 5% w/v, at least about 6% w/v, at least about 7% w/v, at least about 8% w/v, at least about 9% w/v, at least about 10% w/v, at least about 11% w/v, at least about 12% w/v, at least about 13% w/v, at least about 14% w/v, at least about 15% w/v, at least about 16% w/v, at least about 17% w/v, at least about 18% w/v, at least about 19% w/v, at least about 20% w/v, at least about 21% w/v, at least about 22% w/v, at least about 23% w/v, at least about 24% w/v, at least about 25% w/v, at least about 26% w/v, at least about 27% w/v, at least about 28% w/v, at least about 29% w/v, at least about 30% w/v, at least about 31% w/v, at least about 32% w/v, at least about 33% w/v, at least about 34% w/v, at least about 35% w/v, at least about 36% w/v, at least about 37% w/v, at least about 38% w/v, at least about 39% w/v, at least about 40% w/v, at least about 41% w/v, at least about 42% w/v, at least about 43% w/v, at least about 44% w/v, at least about 45% w/v, at least about 46% w/v, at least about 47% w/v, at least about 48% w/v, at least about 49% w/v, at least about 50% w/v, at least about 51% w/v, at least about 52% w/v, at least about 53% w/v, at least about 54% w/v, at least about 55% w/v, at least about 56% w/v, at least about 57% w/v, at least about 58% w/v, at least about 59% w/v, at least about 60% w/v, at least about 61% w/v, at least about 62% w/v, at least about 63% w/v, at least about 64% w/v, at least about 65% w/v, at least about 66% w/v, at least about 67% w/v, at least about 68% w/v, at least about 69% w/v, at least about 70% w/v, at least about 71% w/v, at least about 72% w/v, at least about 73% w/v, at least about 74% w/v, at least about 75% w/v, at least about 76% w/v, at least about 77% w/v, at least about 78% w/v, at least about 79% w/v, at least about 80% w/v, at least about 81% w/v, at least about 82% w/v, at least about 83% w/v, at least about 84% w/v, at least about 85% w/v, at least about 86% w/v, at least about 87% w/v, at least about 88% w/v, at least about 89% w/v, at least about 90% w/v, at least about 91% w/v, at least about 92% w/v, at least about 93% w/v, at least about 94% w/v, or at least about 95% w/v.

In some exemplary embodiments, the aqueous formulation may comprise a mixture of cyclodextrins.

In some exemplary embodiments, the aqueous formulation can further comprise pharmaceutically acceptable excipient(s). Non-limiting examples of excipients can be found in the art (REMINGTON THE SCIENCE AND PRACTICE OF PHARMACY (1990)). Non-limiting examples of excipients can include diluents; acceptable carriers; co-solvents; stabilizers; buffers; emulsifiers; antioxidants including ascorbic acid, methionine, Vitamin E, sodium metabisulfite; polymer; preservatives; isotonicity modifiers; sweetening agents; metal surface active agents; viscosity modifiers; coloring agents; flavoring agents; complexes (e.g., Zn-protein complexes); chelating agents such as EDTA and/or non-ionic surfactants.

In some exemplary embodiments, the aqueous formulation further comprises a stabilizer. In some embodiments, the stabilizer reduces or prevent precipitation of tofacitinib from the aqueous formulation. In some embodiments, the stabilizer reduces or prevent degradation of tofacitinib. In some embodiments, the stabilizer stabilizes the pH of the aqueous formulation within a certain range as described in the present disclosure (e.g. pH of from about 6 to about 8). In some embodiments, the stabilizer reduces or prevent precipitation of tofacitinib from the aqueous formulation, and reduces or prevent degradation of tofacitinib. In some embodiments, the stabilizer reduces or prevent degradation of tofacitinib, and stabilizes the pH of the aqueous formulation within a certain range as described in the present disclosure (e.g. pH of from about 6 to about 8). In some embodiments, the stabilizer reduces or prevent precipitation of tofacitinib from the aqueous formulation, and stabilizes the pH of the aqueous formulation within a certain range as described in the present disclosure (e.g. pH of from about 6 to about 8). In some embodiments, the stabilizer reduces or prevent precipitation of tofacitinib from the aqueous formulation, reduces or prevent degradation of tofacitinib, and stabilizes the pH of the aqueous formulation within a certain range as described in the present disclosure (e.g. pH of from about 6 to about 8).

In some embodiments, the stabilizer is tromethamine (TRIS). TRIS, or tris(hydroxymethyl)aminomethane, or known during medical use as tromethamine or THAM, is an organic compound with the formula (HOCH2)3CNH2. While it is generally used in biochemistry and molecular biology as a component of buffer solutions such as in TAE and TBE buffers, the present disclosure unexpectedly discovered that tromethamine can also be used to stabilize the tofacitinib formulations disclosed herein even when, in some embodiments, its function as a buffering agent becomes less significant due to the lower water content in the formulation.

In some embodiments, the stabilize is a polymer, such as a water-soluble polymer. Moreover, said polymer can be a viscosity enhancing polymer. The term “viscosity enhancing polymer” can mean a polymer that increases the viscosity of a liquid. The polymer increases the viscosity of the aqueous formulation of the disclosure. The increase of viscosity can result in enhanced physical stability of the aqueous formulation. As such, the aqueous formulation can be less prone to sedimentation of the solid complex when it comprises a polymer. The polymer may thus be considered as a polymeric stabilizing agent.

In some exemplary embodiments, the polymer can be a surface active polymer. The term “surface active polymer” can mean a polymer that exhibits surfactant properties. Surface active polymers may, for example, comprise hydrophobic chains grafted to a hydrophilic backbone polymer; hydrophilic chains grafted to a hydrophobic backbone; or alternating hydrophilic and hydrophobic segments. The first two types are called graft copolymers and the third type can be named block copolymer.

In some exemplary embodiments, the aqueous formulation of the disclosure can comprise a polymer selected from the group consisting of a polyoxyethylene fatty acid ester; a polyoxyethylene alkylphenyl ether; a polyoxyethylene alkyl ether; a cellulose derivative such as alkyl cellulose, hydroxyalkyl cellulose and hydroxyalkyl alkylcellulose; a carboxyvinyl polymer such as a carbomer, for example Carbopol 971 and/or Carbopol 974; a polyvinyl polymer; a polyvinyl alcohol; a polyvinylpyrrolidone; a copolymer of polyoxypropylene and polyoxyethylene; tyloxapol; and combinations thereof. Non-limiting examples of suitable polymers include polyethylene glycol monostearate, polyethylene glycol distearate, Hydroxypropyl methylcellulose, hydroxypropylcellulose, polyvinylpyrrolidone, polyoxyethylene lauryl ether, polyoxyethylene octyldodecyl ether, polyoxyethylene stearyl ether, polyoxyethylene myristyl ether, polyoxyethylene oleyl ether, sorbitan esters, polyoxyethylene hexadecyl ether (e.g., cetomacrogol 1000), polyoxyethylene castor oil derivatives, polyoxyethylene sorbitan fatty acid esters (e.g., Tween 20 and Tween 80 (ICI Specialty Chemicals)); polyethylene glycols (e.g., Carbowax 3550 and 934 (Union Carbide)), polyoxyethylene stearates, carboxymethylcellulose calcium, carboxymethylcellulose sodium, methylcellulose, hydroxyethylcellulose, hydroxypropyl methylcellulose, cellulose, polyvinyl alcohol (PVA), poloxamers (e.g., Pluronics F68 and F108, which are block copolymers of ethylene oxide and propylene oxide); poloxamines (e.g., Tetronic 908, also known as Poloxamine 908, which can be a tetrafunctional block copolymer derived from sequential addition of propylene oxide and ethylene oxide to ethylenediamine (BASF Wyandotte Corporation, Parsippany, N. J.)); Tetronic 1508 (T-1508) (BASF Wyandotte Corporation), Tritons X-200, which can be an alkyl aryl polyether sulfonate (Rohm and Haas); PEG-derivatized phospholipid, PEG-derivatized cholesterol, PEG-derivatized cholesterol derivative, PEG-derivatized vitamin A, PEG-derivatized vitamin E, random copolymers of vinyl pyrrolidone and vinyl acetate, combinations thereof and the like. Particularly preferred examples of polymers according to the disclosure are tyloxapol and a copolymer of polyoxypropylene and polyoxyethylene. More particularly, the copolymer of polyoxypropylene and polyoxyethylene may be a triblock copolymer comprising a hydrophilic blockhydrophobic block-hydrophilic block configuration.

In some exemplary embodiments, the polymer can be a poloxamer. Poloxamers can include any type of poloxamer known in the art. Non-limiting examples of Poloxamers include poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, poloxamer 105 benzoate and poloxamer 182 dibenzoate. Poloxamers are also referred to by their trade name Pluronic such as Pluronic 1 ORS, Pluronic 17R2, Pluronic 17R4, Pluronic 25R2, Pluronic 25R4, Pluronic 31 R 1, Pluronic F 108, Pluronic F 108, Pluronic F 108, Pluronic F 108NF, Pluronic F 127, Pluronic F 127 NF, Pluronic F 127, Pluronic F 127, Pluronic F 38, Pluronic F 38, Pluronic F 68, Pluronic F 77, Pluronic F 87, Pluronic F 88, Pluronic F 98, Pluronic L 10, Pluronic L 101, Pluronic L 121, Pluronic L 31, Pluronic L 35, Pluronic L 43, Pluronic L 44, Pluronic L 61, Pluronic L 62, Pluronic L 62 LF, Pluronic L 620, Pluronic L 64, Pluronic L 81, Pluronic L 92, Pluronic L 44, Pluronic N 3, Pluronic P 103, Pluronic P 104, Pluronic P 105, Pluronic P 123, Pluronic P 65, Pluronic P 84, Pluronic P 85, combinations thereof and the like.

In some other exemplary embodiments, a polymeric stabilizing agent compatible with the aqueous formulations and methods can be used. In specific exemplary embodiments, said polymeric stabilizing agent can be tyloxapol. In preferred embodiments, the stabilizer and co-solubilizer can be tyloxapol, which can be a 4-(1, 1,3,3-tetramethylbutyl)phenol polymer with formaldehyde and oxirane.

In some other exemplary embodiments, the viscosity modifiers can be selected from a group consisting of methylcellulose (MC), hydroxypropylmethyl cellulose (HPMC), hydroxyethyl cellulose (HEC), Carbopol®, Pemulin®, Noveon®, polyvinyl alcohol, polyethylene glycol, polyoxyethylene polyoxypropylene glycol (PEPPG), hyaluronic acid salts such as sodium hyaluronate, and polyvinyl pyrrolidone; surfactants such as a polyoxyethylene sorbitan esters and their derivatives (for example, Polysorbate® 80), polyoxyl 40 stearate, polyoxyl 40 hydrogenated castor oil, mixtures thereof, and the like. Some of these agents may have more than one function in the treatment aqueous formulations of the present application; for example, agents such as CMC, HPMC, Pemulin® and Carbopol® are viscosity enhancing agents, but may also function as emulsion stabilizers.

Excipients employed in the aqueous formulations of the present invention can perform two or more functions in the presently useful aqueous formulations. For example, as indicated above, carboxymethylcellulose (CMC), HPMC, Pemulin® and Carbopol® are viscosity enhancing agents, but may also function as emulsion stabilizers. For example, components that are effective as both stabilizers and surfactants may be employed, and/or components that are effective as both polyelectrolyte components and viscosity inducing components may be employed. The specific treatment aqueous formulation chosen for use in the treatment of a given patient in the present invention advantageously can be selected taking into account various factors present in the specific application at hand, for example, the desired treatment of the patient to be achieved, the desired properties of the aqueous formulations to be employed, for example, taking into account the sensitivities of the patient to whom the aqueous formulation can be to be administered, and the like factors.

As used herein, the term “Carbopol®” can refer to the acrylic acid/ethyl acrylate copolymers and the carboxyvinyl polymers sold by the B.F. Goodrich Company under the trademark of Carbopol Registered™ resins. These polymers can be high molecular weight, crosslinked polyacrylic acid polymers. However, the polymers differ by crosslink density and can be grouped into the following categories (i) Carbopol® homopolymers and (ii) Carbopol® copolymers. Non-limiting examples can include Carbopol® 71G, Carbopol® 971P, Carbopol® 974P, Carbopol® 980, Carbopol® 981, Carbopol® 5984, Carbopol® 934, Carbopol® 934P, Carbopol® 940; Carbopol® 941; Carbopol 950, Carbopol 980, Carbopol 951 and Carbopol 981, Carbopol® 5984, and a combination of any two or more of the foregoing. Also suitable for use herein are carbomers sold under the Trade Name Carbopol Ultrez 10, Carbopol ETD2020, Carbopol 1382, Carbopol 1342, Salcare SC96 (Polyquaternium-37 and Propylene Glycol Dicaprylate/Dicaprate and PPG-1 Trideceth-6), Stabileze QM (Polyvinylmethacrylate/Methacrylic acid Decadiene crosspolymer), Stabylen 30 (acrylate/vinyl isodecanoate crosspolymer) and Pemulen TR-1 (CTFA Designation: Acrylates/10-30 Alkyl Acrylate Crosspolymer). Combination of the above polymers are also useful herein.

In some exemplary embodiments, the polymer can be a Carbopol homopolymer. As used herein, the term “Carbopol homopolymers” are polymers of acrylic acid crosslinked with allyl sucrose or allyl pentaerythritol. Non-limiting examples of Carbopol homopolymers can include 71G NF, 971P NF, 974P NF, 980 NF, 981 NF, 5984 EP, 934 NF, 934P NF, 940 NF, and 941 NF. The differences between some of these Carbopol homopolymers are particle size and density, for example, Carbopol 71G, Carbopol 971P and Carbopol 974NF are all allyl ethers of pentaerythritol wherein the residual solvent is ethyl acetate and are referred to as homopolymer type A, homopolymer type A, and homopolymer type B, respectively.

In some exemplary embodiments, the polymer can be a Carbopol copolymer. As used herein, the term “Carbopol copolymer” are polymers of acrylic acid and C₁₀-C₃₀ alkyl acrylate crosslinked with allyl pentaerythritol.

In some exemplary embodiments, the polymer can be a Carbopol interpolymer. As used herein, the term “Carbopol interpolymer” can include a carbomer homopolymer or copolymer that contains a block copolymer of polyethylene glycol and a long chain alkyl acid ester. Non-limiting examples of Carbopol interpolymer can be ETD 2020 NF, Ultrez 10 NF or combination of the two.

In some exemplary embodiments, the concentration of polymer as described above can range from about 0.05% w/w to about 3.0 w/w. In a specific embodiment, the concentration of polymer can range be about 0.05% w/w, about 0.05% w/w, 0.06% w/w, about 0.07% w/w, 0.08% w/w, about 0.09% w/w, 0.10% w/w, about 0.15% w/w, about 0.20% w/w, about 0.25% w/w, about 0.30% w/w, about 0.35% w/w, about 0.40% w/w, about 0.45% w/w, about 0.50% w/w, about 0.55% w/w, about 0.60% w/w, about 0.65% w/w, about 0.70% w/w, about 0.75% w/w, about 0.80% w/w, about 0.85% w/w, about 0.90% w/w, about 0.95% w/w, about 1.0% w/w, about 1.05%, about 1.10% w/w, about 1.15% w/w, about 1.20% w/w, about 1.25% w/w, about 1.30% w/w, about 1.35% w/w, about 1.40% w/w, about 1.45% w/w, about 1.50% w/w, about 1.55% w/w, about 1.60% w/w, about 1.65% w/w, about 1.70% w/w, about 1.75% w/w, about 1.80% w/w, about 1.85% w/w, about 1.90% w/w, about 1.95% w/w, about 2.0% w/w, about 2.05%, about 2.10% w/w, about 2.15% w/w, about 2.20% w/w, about 2.25% w/w, about 2.30% w/w, about 2.35% w/w, about 2.40% w/w, about 2.45% w/w, about 2.50% w/w, about 2.55% w/w, about 2.60% w/w, about 2.65% w/w, about 2.70% w/w, about 2.75% w/w, about 2.80% w/w, about 2.85% w/w, about 2.90% w/w, about 2.95% w/w, or about 3.0% w/w.

When the aqueous formulation comprises a polymer, the viscosity of the aqueous formulation can range from about 100 to about 4000 cPs (measured using a Brookfield Viscometer). In some exemplary embodiments, the viscosity of the aqueous formulation be about 100, about 200 cP, about 300 cP, about 400 cP, about 500 cP, about 600, about 700 cP, about 800 cP, about 900 cP, about 1000 cP, 1100, about 1200 cP, about 1300 cP, about 1400 cP, about 1500 cP, about 1600, about 700 cP, about 800 cP, about 900 cP, about 2000 cP, 2100, about 2200 cP, about 2300 cP, about 2400 cP, about 2500 cP, about 2600, about 2700 cP, about 2800 cP, about 2900 cP, about 3000 cP, 3100, about 3200 cP, about 3300 cP, about 3400 cP, about 3500 cP, about 3600, about 3700 cP, about 3800 cP, about 3900 cP, or about 4000 cP.

As used herein, the term “viscosity” can be a ratio of shear stress to shear rate, expressed as dynes-second/cm², or poise. A centipoise (cps) is one hundredth of a poise. A poise is a unit of coefficient of viscosity, defined as the tangential force per unit area required to maintain one unit difference in velocity between two parallel planes separated by one centimeter of fluid. Any viscosity determination should be carried out using a Brookfield Viscometer at room temperature. The viscosity can be measured by operating the viscometer at a spindle speed that is the highest speed possible to obtain a reading that is on scale.

Other excipients suitable for use herein include oleogels such as trihydroxystearin and aluminum magnesium hydroxy stearate. Another useful thickener for the present invention can be the non-ionic polymer under the CTFA designation: polyacrylamide and isoparrafin and laureth-7, available as Sepigel from Seppic Corporation. Also suitable for use herein are carbomers sold under the Trade Name Carbopol Ultrez 10, Carbopol ETD2020, Carbopol 1382, Carbopol 1342, Salcare SC96 (Polyquaternium-37 and Propylene Glycol Dicaprylate/Dicaprate and PPG-1 Trideceth-6), Stabileze QM (Polyvinylmethacrylate/Methacrylic acid Decadiene crosspolymer), Stabylen 30 (acrylate/vinyl isodecanoate crosspolymer) and Pemulen TR-1 (CTFA Designation: Acrylates/10-30 Alkyl Acrylate Crosspolymer).

In some exemplary embodiments, Hydrophobically modified celluloses can be further present in the aqueous formulation. These celluloses are described in detail in U.S. Pat. Nos. 4,228,277 and 5,104,646, both of which are herein incorporated by reference in their entirety.

In some exemplary embodiments, the aqueous formulation can comprise of a thickener at a concentration of from about 0.01% to about 10%, preferably from about 0.1% to about 5%, more preferably from about 0.1% to about 1% and even more preferably from about 0.1% to about 0.5%.

In some exemplary embodiments, the aqueous formulation of the disclosure can comprise a pharmaceutically acceptable medium. The term “pharmaceutically acceptable medium” as used herein can mean a medium suitable for administration of the aqueous formulation to a mammalian subject, such as a human. The pharmaceutically acceptable medium notably comprises an aqueous medium. In some exemplary embodiments, the pharmaceutically acceptable medium can be water, water for injection, pH-adjusted water or a suitable buffer.

According to a preferred embodiment the pharmaceutically acceptable medium comprises water and optionally an additive selected from the group consisting of a preservative, a stabilizing agent, an electrolyte, a buffering agent, and combinations thereof.

In particular, the pharmaceutically acceptable medium may comprise a preservative. A preservative may be used to limit bacterial proliferation in the aqueous formulation.

Suitable examples of preservative are sodium bisulfite, sodium bisulfate, sodium thiosulfate, benzalkonium chloride, chlorobutanol, thimerosal, phenylmercuric acetate, phenylmercuric nitrate, methylparaben, phenylethyl alcohol, and combinations thereof. Preferably, the preservative can be benzalkonium chloride.

The amount of preservative in the aqueous formulation of the disclosure may be 0 to 1%, in particular 0.001 to 0.5%, more particularly 0.005 to 0.1%, even more particularly 0.01 to 0.04%, by weight of preservative based on the volume of the aqueous formulation.

In particular, the acceptable medium may comprise an additional stabilizing agent, which may be used to reduce degradation or stabilize the aqueous formulation during storage.

An example of a suitable stabilizing agent can be disodium edetate.

The amount of stabilizing agent in the aqueous formulation of the disclosure may be 0 to 1%, in particular 0.01 to 0.5%, more particularly 0.08 to 0.2% by weight of stabilizing agent based on the volume of the aqueous formulation.

In particular, the pharmaceutically acceptable medium may comprise an electrolyte. An electrolyte may especially be used to make the aqueous formulation isotonic.

Examples of suitable electrolytes include sodium chloride, potassium chloride, and combinations thereof. Preferably, the electrolyte can be sodium chloride.

The amount of electrolyte in the aqueous formulation of the disclosure may be 0 to 2%, in particular 0.1 to 1.5%, more particularly 0.5 to 1% by weight of electrolyte based on the volume of the aqueous formulation.

In some exemplary embodiments, the pH of the aqueous formulation as described herein can range from about 3.6 to about 8.0. In some specific exemplary embodiments, the pH of the aqueous formulation can be about 3.6, about 3.7, about 3.8, about 3.9, about 4, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0. In a specific exemplary embodiment, the pH of the aqueous formulation can be in the range of about 4.5 to about 7.5, about 5 to about 8, or about 5 to about 7.

In some exemplary embodiments, the API can be substantially dissolved in the aqueous formulation. As used herein, the term “dissolved” or “substantially dissolved” can mean the solubilization of a solid in a solution. It can be considered that a solid can be “dissolved” or “substantially dissolved” in a solution when the resulting solution can be clear or substantially clear.

In some exemplary embodiments, the aqueous formulation can be clear or substantially clear. As used herein, the term “clear” can mean a translucent or a sub-translucent solution. Thus, a “clear” solution has a turbidity measured according to ISO standards of <100 Nephelometric Turbidity Units (NTUs), preferably <50 NTUs.

As used herein, the term “substantially clear” can mean a translucent or a sub-translucent solution. Thus, a “substantially clear” solution has a turbidity measured according to ISO standards of 100 Nephelometric Turbidity Units (NTUs).

In some exemplary embodiments, the aqueous formulation can be cloudy or substantially cloudy. As used herein, the term “cloudy” or “substantially cloudy” or refers to a solution having a turbidity measured according to ISO standards of greater than 100 NTUs.

In some exemplary embodiments, the aqueous formulation can be milky or substantially milky. As used herein, the term “milky” or “substantially milky” refers to a solution having a turbidity measured according to ISO standards of greater than 100 NTUs, preferably greater than 200 NTUs.

In one exemplary embodiment, at least about 90% of the aqueous formulation can be a solution, a nano-suspension or a combination thereof. In one aspect of this embodiment, the amount of aqueous formulation that can be a solution, a nano-suspension or a combination thereof can be at least about 90%, at least about 92%, at least about 93%, at least about 94%, at least about 95%, at least about 96%, at least about 97%, at least about 98%, at least about 99.1%, at least about 99.2%, at least about 99.3%, at least about 99.4%, at least about 99.5%, at least about 99.6%, at least about 99.7%, at least about 99.8%, or at least about 99.9%.

In some exemplary embodiments, at least about 95% of the aqueous formulation can be a solution, a nano-suspension or a combination thereof.

As used herein, the term “nanosuspension” can mean an aqueous formulation comprising nanoparticles. The term “nanoparticle” refers to a particle having a D50 of less than about 1 μm.

In one exemplary embodiment, at most about 10% of the aqueous formulation can be a micro-suspension. In one aspect of this embodiment, the amount of aqueous formulation that can be a micro-suspension can be at most about 0.1%, at most about 0.2%, at most about 0.3%, at most about 0.4%, or at most about 0.5%, at most about 0.6%, at most about 0.7%, at most about 0.8%, at most about 0.9%, at most about 1%, at most about 2%, at most about 3%, at most about 4%, at most about 5%, at most about 6%, at most about 7%, at most about 8%, at most about 9%, or at most about 10%. In another aspect of this embodiment, the amount of aqueous formulation that can be a micro-suspension can be negligible.

The term “micro-suspension” as used herein can mean an aqueous formulation comprising solid complex microparticles suspended in a liquid phase. As used herein the term “microparticle” can refer to a particle having a diameter D50 of 1 μm or greater to about 200 m. The term “nanoparticle” refers to a particle having a diameter D50 of less than 1 μm.

In some exemplary embodiments, at least about 90% of the aqueous formulation passes through a 0.2 micron filter.

In some exemplary embodiments, the viscosity of the aqueous formulation can be less than about 4000 cP. In some one specific embodiment, the viscosity of the aqueous formulation can be less than about 100 cP, less than about 200 cP, less than about 300 cP, less than about 400 cP, less than about 500 cP, less than about 600 cP, less than about 700 cP, less than about 800 cP, less than about 900 cP, less than about 1000 cP, less than about 1100 cP, less than about 1200 cP, less than about 1300 cP, less than about 1400 cP, less than about 1500 cP, less than about 1600 cP, less than about 1700 cP, less than about 1800 cP, less than about 900 cP, less than about 2000 cP, less than about 2100 cP, less than about 2200 cP, less than about 2300 cP, less than about 2400 cP, less than about 2500 cP, less than about 2600 cP, less than about 2700 cP, less than about 2800 cP, less than about 2900 cP, less than about 3000 cP, less than about 3100 cP, less than about 3200 cP, less than about 3300 cP, less than about 3400 cP, less than about 3500 cP, less than about 3600 cP, less than about 3700 cP, less than about 3800 cP, less than about 3900 cP, or less than about 4000 cP (measured using a Brookfield Viscometer).

In some exemplary embodiments, the disclosure provides a method of preparing an aqueous formulation comprising an active pharmaceutical ingredient and cyclodextrin. In some exemplary embodiments, the disclosure provides a method of preparing an aqueous formulation comprising an active pharmaceutical ingredient and cyclodextrin. The aqueous formulations of the disclosure can be obtainable by or obtained by the following methods. All of the embodiments, preferred recitations and particular examples cited in the previous sections equally apply to the methods of the disclosure and the aqueous formulations obtained with the methods of the disclosure.

In some exemplary embodiments, a method of preparing an aqueous formulation can comprise adding tofacitinib or its acid addition salt in an aqueous medium. In some specific exemplary embodiments, the aqueous formulation can be stirred until all solids are dissolved to afford a clear viscous liquid. In some specific exemplary embodiments, the aqueous formulation can be further filtered via 0.2 μm filter. In some specific exemplary embodiments, the pH of the aqueous formulation can be adjusted to a pH of about 3.6 to about 8.0, about 5.0 to about 7.0, or about 5.0 to about 6.5. In some specific exemplary embodiments, the pH of the aqueous formulation can be adjusted to a pH of about 6.0 to 8.0. In some specific exemplary embodiments, the pH of the aqueous formulation can be adjusted using NaOH or HCl. In some specific exemplary embodiments, the concentration of tofacitinib free base or its acid addition salt can be at least about 20 mg/mL, at least about 25 mg/mL, or at least about 30 mg/mL. In some specific exemplary embodiments, the aqueous formulation can be heated up to about 60° C.

In some exemplary embodiments, a method of preparing an aqueous formulation can comprise adding tofacitinib or its acid addition salt in an aqueous medium comprising cyclodextrin. In some specific exemplary embodiments, the aqueous formulation can be stirred until all solids are dissolved to afford a clear viscous liquid. In some specific exemplary embodiments, the aqueous formulation can be further filtered via 0.2 μm filter. In some specific exemplary embodiments, the pH of the aqueous formulation can be adjusted to a pH of about 3.6 to about 8.0. In some specific exemplary embodiments, the concentration of tofacitinib free base or its acid addition salt can be at least about 20 mg/mL, at least about 25 mg/mL, or at least about 30 mg/mL. Any of the cyclodextrin described herein can be used to prepare the aqueous formulation. In some specific exemplary embodiments, the concentration of the cyclodextrin can be 10% w/w to about 90% w/w. In some specific exemplary embodiments, the aqueous formulation can be heated up to about 60° C.

In some exemplary embodiments, a method of preparing an aqueous formulation can comprise adding tofacitinib or its acid addition salt in an aqueous medium comprising cyclodextrin and a stabilizer. In some specific exemplary embodiments, the aqueous formulation can be stirred until all solids are dissolved to afford a clear viscous liquid. In some specific exemplary embodiments, the aqueous formulation can be further filtered via 0.2 μm filter. In some specific exemplary embodiments, the pH of the aqueous formulation can be adjusted to a pH of about 3.6 to about 8.0. In some specific exemplary embodiments, the pH of the aqueous formulation be adjusted to a pH of about 3.6, about 3.7, about 3.8, about 3.9, about 4, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0. In a specific exemplary embodiment, the pH of the aqueous formulation can be in the range of about 4.5 to about 7.5, about 5 to about 8, or about 5 to about 7.

In some specific exemplary embodiments, the concentration of tofacitinib free base or its acid addition salt can be at least about 20 mg/mL, at least about 25 mg/mL, or at least about 30 mg/mL. Any of the cyclodextrin described herein can be used to prepare the aqueous formulation. In some specific exemplary embodiments, the concentration of the cyclodextrin can be 10% w/w to about 90% w/w. Any of the stabilizer described herein can be used to prepare the aqueous formulation. In some specific exemplary embodiments, the concentration of the stabilizer can be 0.05% w/w to about 3.0% w/w. In some specific exemplary embodiments, the aqueous formulation can be heated up to about 60° C.

In some exemplary embodiments, a method of preparing an aqueous formulation can comprise adding tofacitinib or its acid addition salt in an aqueous medium comprising cyclodextrin and/or a stabilizer followed by adding an additional excipient. In some specific exemplary embodiments, the aqueous formulation can be stirred until all solids are dissolved to afford a clear viscous liquid. In some specific exemplary embodiments, the aqueous formulation can be heated up to about 60° C. In some specific exemplary embodiments, the aqueous formulation can be further filtered via 0.2 μm filter. In some specific exemplary embodiments, the pH of the aqueous formulation can be adjusted to a pH of about 3.6 to about 8.0. In some specific exemplary embodiments, the concentration of tofacitinib free base or its acid addition salt can be at least about 20 mg/mL, at least about 25 mg/mL, or at least about 30 mg/mL. Any of the cyclodextrin described herein can be used to prepare the aqueous formulation. In some specific exemplary embodiments, the concentration of the cyclodextrin can be 10% w/w to about 90% w/w. Any of the stabilizer described herein can be used to prepare the aqueous formulation. In some specific exemplary embodiments, the concentration of the stabilizer can be 0.05% w/w to about 3.0% w/w. Any of the excipients described herein can be used to prepare the aqueous formulation. In some specific exemplary embodiments, more than one excipient can be sued to prepare the aqueous formulation.

In some exemplary embodiments, the disclosure provides a method of treating a disease or condition in a subject in need by administering to the subject an aqueous formulation as by any of the embodiments described herein.

In some exemplary embodiments, the disease can be a disease of the gastrointestinal (GI) tract. Non-limiting examples of gastrointestinal tract diseases that can be treated include, without limitation, inflammatory bowel disease (IBD), Crohn's disease (e.g., active Crohn's disease, refractory Crohn's disease, or fistulizing Crohn's disease), ulcerative colitis, indeterminate colitis, microscopic colitis, infectious colitis, drug or chemical-induced colitis, diverticulitis, and ischemic colitis, gastritis, peptic ulcers, stress ulcers, bleeding ulcers, gastric hyperacidity, dyspepsia, gastroparesis, Zollinger-Ellison syndrome, gastroesophageal reflux disease, short-bowel (anastomosis) syndrome, a hypersecretory state associated with systemic mastocytosis or basophilic leukemia or hyperhistaminemia, Celiac disease (e.g., nontropical Sprue), enteropathy associated with seronegative arthropathies, microscopic colitis, collagenous colitis, eosinophilic gastroenteritis, colitis associated with radiotherapy or chemotherapy, colitis associated with disorders of innate immunity as in leukocyte adhesion deficiency-1, chronic granulomatous disease, food allergies, gastritis, infectious gastritis or enterocolitis (e.g., Helicobacter pylori-infected chronic active gastritis), other forms of gastrointestinal inflammation caused by an infectious agent, pseudomembranous colitis, hemorrhagic colitis, hemolytic-uremic syndrome colitis, diversion colitis, irritable bowel syndrome, irritable colon syndrome, and pouchitis.

Examples of therapeutic applications of the aqueous formulations described herein are illustrated in WO2019/036382 which is being incorporated by reference in their entirety.

In some exemplary embodiments, the disease or condition can be a gastrointestinal inflammatory disorder.

As used herein, the term “Gastrointestinal inflammatory disorders” can refer to a group of chronic disorders that cause inflammation and/or ulceration in the mucous membrane. These disorders include, for example, inflammatory bowel disease (e.g., Crohn's disease, ulcerative colitis, indeterminate colitis and infectious colitis), mucositis (e.g., oral mucositis, gastrointestinal mucositis, nasal mucositis and proctitis), necrotizing enterocolitis and esophagitis.

In some exemplary embodiments, the disease of the GI tract can be an inflammatory bowel disorder.

As used herein, the term “Inflammatory Bowel Disease” or “IBD” can refer to a chronic inflammatory autoimmune condition of the gastrointestinal (GI) tract. The GI tract can be divided into four main different sections, the oesophagus, stomach, small intestine and large intestine or colon. The small intestine possesses three main subcompartments: the duodenum, jejunum and ileum. Similarly, the large intestine consists of six sections: the cecum, ascending colon, transverse colon, ascending colon, sigmoid colon, and the rectum. The small intestine is about 6 m long, its diameter is about 2.5 to 3 cm and the transit time through it is typically about 3 hours. The duodenum has a C-shape, and is about 30 cm long. Due to its direct connection with the stomach, it is physically more stable than the jejunum and ileum, which are sections that can freely move. The jejunum is about 2.4 m in length and the ileum is about 3.6 m in length and their surface areas are about 180 m² and about 280 m² respectively. The large intestine is 1.5 m long, its diameter is between about 6.3 and about 6.5 cm, the transit time though this section is about 20 hours and has a reduced surface area of about 150 m². The higher surface area of the small intestine enhances its capacity for systemic drug absorption.

The etiology of IBD is complex, and many aspects of the pathogenesis remain unclear. The treatment of moderate to severe IBD poses significant challenges to treating physicians, because conventional therapy with corticosteroids and immunomodulator therapy (e.g., azathioprine, 6-mercaptopurine, and methotrexate administered via traditional routes such as tablet form, oral suspension, or intravenously) is associated with side effects and intolerance and has not shown proven benefit in maintenance therapy (steroids). Monoclonal antibodies targeting tumor necrosis factor alpha (TNF-α), such as infliximab (a chimeric antibody) and adalimumab (a fully human antibody), are currently used in the management of CD. Infliximab has also shown efficacy and has been approved for use in UC. However, approximately 10%-20% of patients with CD are primary nonresponders to anti TNF therapy, and another ˜20%-30% of CD patients lose response over time (F Schnitzler et al., Long-term outcome of treatment with infliximab in 614 patients with Crohns disease: results from a single-centre cohort, 58 GUT 492-500 (2008)). Other adverse events (AEs) associated with anti TNFs can include elevated rates of bacterial infection, including tuberculosis, and, more rarely, lymphoma and demyelination (John T Chang & Gary R Lichtenstein, Drug Insight: antagonists of tumor-necrosis factor-α in the treatment of inflammatory bowel disease, 3 NATURE CLINICAL PRACTICE GASTROENTEROLOGY & HEPATOLOGY 220-228 (2006); Frank Hoentjen & Ad A Van Bodegraven, Safety of anti-tumor necrosis factor therapy in inflammatory bowel disease, 15 WORLD JOURNAL OF GASTROENTEROLOGY 2067 (2009)). In addition, most patients do not achieve sustained steroid-free remission and mucosal healing, clinical outcomes that correlate with true disease modification.

Although the cause of IBD remains unknown, several factors such as genetic, infectious and immunologic susceptibility have been implicated. IBD is much more common in Caucasians, especially those of Jewish descent. The chronic inflammatory nature of the condition has prompted an intense search for a possible infectious cause. Although agents have been found which stimulate acute inflammation, none has been found to cause the chronic inflammation associated with IBD. The hypothesis that IBD is an autoimmune disease is supported by the previously mentioned extraintestinal manifestation of IBD as joint arthritis, and the known positive response to IBD by treatment with therapeutic agents such as adrenal glucocorticoids, cyclosporine and azathioprine, which are known to suppress immune response. In addition, the GI tract, more than any other organ of the body, is continuously exposed to potential antigenic substances such as proteins from food, bacterial byproducts (LPS), etc.

In some exemplary embodiments, the disease of the GI tract can be autoimmune condition. A chronic inflammatory autoimmune condition of the gastrointestinal (GI) tract can present clinically as either ulcerative colitis (UC) or Crohn's disease. Both IBD conditions are associated with an increased risk for malignancy of the GI tract.

In some exemplary embodiments, the disease of the GI tract can be Crohn's disease. In some specific exemplary embodiments, the disease of the GI tract can be ileal Crohn's disease.

As used herein, the term “Crohn's disease” can refer to a chronic transmural inflammatory disease with the potential to affect any part of the entire GI tract. Crohn's disease can be frequently complicated by the development of malabsorption, strictures, and fistulae and may require repeated surgery. UC, less frequently, may be complicated by severe bloody diarrhea and toxic megacolon, also requiring surgery. The most prominent feature Crohn's disease is the granular, reddish-purple edematous thickening of the bowel wall. With the development of inflammation, these granulomas often lose their circumscribed borders and integrate with the surrounding tissue. Diarrhea and obstruction of the bowel are the predominant clinical features. As with ulcerative colitis, the course of Crohn's disease may be continuous or relapsing, mild or severe, but unlike ulcerative colitis, Crohn's disease is not curable by resection of the involved segment of bowel. Most patients with Crohn's disease require surgery at some point, but subsequent relapse is common and continuous medical treatment is usual. Crohn's disease may involve any part of the alimentary tract from the mouth to the anus, although typically it appears in the ileocolic, small-intestinal or colonic-anorectal regions. Histopathologically, the disease manifests by discontinuous granulomatomas, crypt abscesses, fissures and aphthous ulcers. The inflammatory infiltrate is mixed, consisting of lymphocytes (both T and B cells), plasma cells, macrophages, and neutrophils. There is a disproportionate increase in IgM- and IgG-secreting plasma cells, macrophages and neutrophils.

To date, the primary outcome measure in Crohn's Disease clinical trials is the Crohn's Disease Activity Index (CDAI), which has served as the basis for approval of multiple drug treatments, including for example, vedolizumab and natalizumab. The CDAI was developed by regressing clinician global assessment of disease activity on eighteen potential items representing patient reported outcomes (PROs) (e.g., abdominal pain, pain awakening patient from sleep, appetite), physical signs (e.g., average daily temperature, abdominal mass), medication use (e.g., loperamide or opiate use for diarrhea) and a laboratory test (e.g., hematocrit). Backward stepwise regression analysis identified eight independent predictors which are the number of liquid or soft stools, severity of abdominal pain, general well-being, occurrence of extra-intestinal symptoms, need for anti-diarrheal drugs, presence of an abdominal mass, hematocrit, and body weight. The final score is a composite of these eight items, adjusted using regression coefficients and standardization to construct an overall CDAI score, ranging from 0 to 600 with higher score indicating greater disease activity. Widely used benchmarks are: CDAI <150 is defined as clinical remission, 150 to 219 is defined as mildly active disease, 220 to 450 is defined as moderately active disease, and above 450 is defined as very severe disease (W R Best et al., Development of a Crohn's disease activity index. National Cooperative Crohn's Disease Study., 77 DEVELOPMENT OF A CROHN'S DISEASE ACTIVITY INDEX. NATIONAL COOPERATIVE CROHN'S DISEASE STUDY. 843-846 (1979)). Vedolizumab and natalizumab have been approved on the basis of demonstrated clinical remission, i.e., CDAI <150.

Although the CDAI has been in use for over 40 years, and has served as the basis for drug approval, it has several limitations as an outcome measure for clinical trials. For example, most of the overall score comes from the patient diary card items (pain, number of liquid bowel movements, and general well-being), which are vaguely defined and not standardized terms (R S Sandler, M C Jordan & L L Kupper, Development of a Crohn's index for survey research., 41 JOURNAL OF CLINICAL EPIDEMIOLOGY 451-458 (1988); Kelvin Thia et al., Short CDAI: Development and validation of a shortened and simplified Crohn's disease activity index, 17 INFLAMMATORY BOWEL DISEASES 105-111 (2011)). In addition, measurement of pain can be based on a four-point scale rather than an updated seven-point scale. The remaining 5 index items contribute very little to identifying an efficacy signal and may be a source of measurement noise. Furthermore, concerns have been raised about poor criterion validity for the CDAI, a reported lack of correlation between the CDAI and endoscopic measures of inflammation (which may render the CDAI as a poor discriminator of active CD and irritable bowel syndrome) and high reported placebo rates (J R Korzenik et al., Sargramostimfor active Crohn's disease., 352 THE NEW ENGLAND JOURNAL OF MEDICINE 2193-2201 (2005); Sandborn et al. 2005, supra; William J. Sandborn et al., Adalimumab Induction Therapy for Crohn Disease Previously Treated with Infliximab, 146 ANNALS OF INTERNAL MEDICINE 829 (2007); Yoonhee Kim et al., Su1083 Can Crohn's Disease Activity Index Differentiate Clinical Remission Induced by Placebo Versus Biologics Treatment?—Analyses of Six Clinical Trials for Crohn's Disease, 146 GASTROENTEROLOGY S-368 (2014)). It is, thus, generally recognized that additional or alternative measures of CD symptoms are needed, such as new PRO tools or adaptations of the CDAI to derive a new PRO. The PRO2 and PRO3 tools are such adaptations of the CDAI and have been recently described in R. Khanna et al., A retrospective analysis: the development of patient reported outcome measures for the assessment of Crohns disease activity, 41 ALIMENTARY PHARMACOLOGY & THERAPEUTICS 77-86 (2014). The PRO2 evaluates the frequency of loose/liquid stools and abdominal pain (Id). These items are derived and weighted accordingly from the CDAI and are the CDAI diary card items, along with general well-being, that contribute most to the observed clinical benefit measured by CDAI (Sandler et al. 1988, supra; Thia et al. 2011, supta; Kim et al. 2014, supra). The remission score of <11 is the CDAI-weighted sum of the average stool frequency and pain scores in a 7-day period, which yielded optimum sensitivity and specificity for identification of CDAI remission (score of <150) in a retrospective data analysis of ustekinumab induction treatment for moderate to severe CD in a Phase II clinical study. The PRO2 was shown to be sensitive and responsive when used as a continuous outcome measure in a retrospective data analysis of MTX treatment in active CD (Reena Khanna et al., A Systematic Review of Measurement of Endoscopic Disease Activity and Mucosal Healing in Crohn's Disease, 20 INFLAMMATORY BOWEL DISEASES 1850-1861 (2014)) measured by CDAI. Additional outcome measures include the Mayo Clinic Score, the Crohn disease endoscopic index of severity (CDEIS), and the Ulcerative colitis endoscopic index of severity (UCEIS). Additional outcome measures include Clinical remission, Mucosal healing, Histological healing (transmural), MRI or ultrasound for measurement or evaluation of bowel wall thickness, abscesses, fistula and histology.

An additional means of assessing the extent and severity of Crohn's Disease is endoscopy. Endoscopic lesions typical of Crohn's disease have been described in numerous studies and include, e.g., aphthoid ulcerations, “punched-out ulcers,” cobblestoning and stenosis. Endoscopic evaluation of such lesions was used to develop the first validated endoscopic score, the Crohn's Disease Endoscopic Index of Severity (CDEIS) (J Y Mary & R Modigliani, Development and validation of an endoscopic index of the severity for Crohn's disease: a prospective multicentre study, 30 GUT 983-989 (1989)). More recently, because the CDEIS is time-consuming, complicated and impractical for routine use, a Simplified Endoscopic Activity Score for Crohn's Disease (SES-CD) was developed and validated (Marco Daperno et al., Development and validation of a new, simplified endoscopic activity score for Crohns disease: the SES-CD, 60 GASTROINTESTINAL ENDOSCOPY 505-512 (2004)). The SES-CD consists of four endoscopic variables (size of ulcers, proportion of surface covered by ulcers, proportion of surface with any other lesions (e.g., inflammation), and presence of narrowings [stenosis]) that are scored in five ileocolonic segments, with each variable, or assessment, rated from 0 to 3.

To date, there is no cure for Crohn's disease. Accordingly, the current treatment goals for Crohn's disease are to induce and maintain symptom improvement, induce mucosal healing, avoid surgery, and improve quality of life (G R Lichtenstein et al., Management of Crohn's disease in adults., 104 THE AMERICAN JOURNAL OF GASTROENTEROLOGY 465-483 (2009); Gert Van Assche et al., The second European evidence-based Consensus on the diagnosis and management of Crohn's disease: Special situations, 4 JOURNAL OF CROHNS AND COLITIS 63-101 (2010)). The current therapy of IBD usually involves the administration of anti-inflammatory or immunosuppressive agents, such as sulfasalazine, corticosteroids, 6-mercaptopurine/azathioprine, or cyclosporine, all of which are not typically delivered by localized release of a drug at the site or location of disease. More recently, biologics like TNF-alpha inhibitors and IL-12/IL-23 blockers, can be used to treat IBD. If anti-inflammatory/immunosuppressive/biologic therapies fail, colectomies are the last line of defense. The typical operation for Crohn's disease not involving the rectum is resection (removal of a diseased segment of bowel) and anastomosis (reconnection) without an ostomy. Sections of the small or large intestine may be removed. About 30% of Crohn's disease patients will need surgery within the first year after diagnosis. In the subsequent years, the rate is about 5% per year. Unfortunately, Crohn's disease is characterized by a high rate of recurrence; about 5% of patients need a second surgery each year after initial surgery.

Refining a diagnosis of inflammatory bowel disease involves evaluating the progression status of the diseases using standard classification criteria. The classification systems used in IBD include the Truelove and Witts Index (S. C. Truelove & L. J. Witts, Cortisone in Ulcerative Colitis, 2 BMJ 1041-1048 (1955), which classifies colitis as mild, moderate, or severe, as well as Lennard-Jones (J E Lennard-Jones, Classification of inflammatory bowel disease., 170 SCANDINAVIAN JOURNAL OF GASTROENTEROLOGY 2-6 (1989)) and the simple clinical colitis activity index (SCCAI). (R S Walmsley et al., A simple clinical colitis activity index, 43 GUT 29-32 (1998)). These systems track such variables as daily bowel movements, rectal bleeding, temperature, heart rate, hemoglobin levels, erythrocyte sedimentation rate, weight, hematocrit score, and the level of serum albumin.

There is sufficient overlap in the diagnostic criteria for UC and Crohn's disease that it is sometimes impossible to say which a given patient has; however, the type of lesion typically seen is different, as is the localization. UC mostly appears in the colon, proximal to the rectum, and the characteristic lesion is a superficial ulcer of the mucosa; Crohn's disease can appear anywhere in the bowel, with occasional involvement of stomach, esophagus and duodenum, and the lesions are usually described as extensive linear fissures.

In approximately 10-15% of cases, a definitive diagnosis of ulcerative colitis or Crohn's disease cannot be made and such cases are often referred to as “indeterminate colitis.” Two antibody detection tests are available that can help the diagnosis, each of which assays for antibodies in the blood. The antibodies are “perinuclear anti-neutrophil antibody” (pANCA) and “anti-Saccharomyces cerevisiae antibody” (ASCA). Most patients with ulcerative colitis have the pANCA antibody but not the ASCA antibody, while most patients with Crohn's disease have the ASCA antibody but not the pANCA antibody. However, these two tests have shortcomings as some patients have neither antibody and some Crohn's disease patients may have only the pANCA antibody. A third test, which measures the presence and accumulation of circulating anti-microbial antibodies—particularly flagellin antibodies, has proven to be useful for detecting susceptibility to Crohn's Disease before disease development. See Choung, R. S., et al. “Serologic microbial associated markers can predict Crohns disease behaviour years before disease diagnosis”, 43 ALIMENTARY PHARMACOLOGY & THERAPEUTICS 1300-1310 (2016).

In some specific exemplary embodiments, the disease of the GI tract can be ulcerative colitis.

As used herein, the term “Ulcerative colitis (UC)” can refer to the condition that afflicts the large intestine. The course of the disease may be continuous or relapsing, mild or severe. The earliest lesion is an inflammatory infiltration with abscess formation at the base of the crypts of Lieberkuhn. Coalescence of these distended and ruptured crypts tends to separate the overlying mucosa from its blood supply, leading to ulceration. Symptoms of the disease include cramping, lower abdominal pain, rectal bleeding, and frequent, loose discharges consisting mainly of blood, pus and mucus with scanty fecal particles. A total colectomy may be required for acute, severe or chronic, unremitting ulcerative colitis.

The clinical features of UC are highly variable, and the onset may be insidious or abrupt, and may include diarrhea, tenesmus and relapsing rectal bleeding. With fulminant involvement of the entire colon, toxic megacolon, a life-threatening emergency, may occur. Extraintestinal manifestations include arthritis, pyoderma gangrenoum, uveitis, and erythema nodosum.

In some exemplary embodiments, aqueous formulations, and methods disclosed herein are used to treat one gastrointestinal disease. In some exemplary embodiments, aqueous formulations, and methods disclosed herein are used to treat more than one gastrointestinal disease. In some exemplary embodiments, aqueous formulations, and methods disclosed herein are used to treat multiple gastrointestinal diseases that occur in the same area of the gastrointestinal tract (e.g., each disease can occur in the small intestine, large intestine, colon, or any sub-region thereof). In some exemplary embodiments, apparatuses, aqueous formulations, and methods disclosed herein are used to treat multiple gastrointestinal diseases that occur in different areas of the gastrointestinal tract. In some exemplary embodiments, administration (e.g., local administration to the gastrointestinal tract) of JAK inhibitor is useful in the treatment of gastrointestinal diseases including, but not limited to, inflammatory bowel disease (IBD), ulcerative colitis, Crohn's disease, or any of the other gastrointestinal diseases described herein.

The aqueous formulation described herein can administered orally. When ingested, an oral aqueous formulation of tofacitinib solution can pass through the stomach, before reaching the gastrointestinal tract. For treatment of disease or condition in the lower gastrointestinal tract, the exposure of tofacitinib in the stomach can cause systemic exposure instead of exposure to the desired area. A targeted delivery can prevent release of the tofacitinib under the acidic conditions prevalent in the stomach.

In some exemplary embodiments, the disclosure provides a method of delivering an aqueous formulation comprising an active pharmaceutical ingredient and cyclodextrin.

In some exemplary embodiments, the route of administration of the aqueous formulation can be auricular, buccal, epidural, intra-arterial, intra-articular, intracardiac, intraductal, intrafollicular, intragastric, intralesional, intramuscular, intramammary, respiratory, intraperitoneal, intrasinal, intrasynovial, intrathecal, intratesticular, intrauterine, intravenous, intravesical, nasal, ophthalmic, parenteral, perineural, oral, vaginal, rectal, subcutaneous, sublingual, topical, dermal or transdermal.

In some exemplary embodiments, the method of delivery can include a topical delivery of the aqueous formulation. As used herein, “topical delivery” can refer to a route of administration of a medicament (e.g., a drug or a pharmaceutical formulation containing a drug) where the medicament is applied to a localized area of the body or to the surface of a body part, regardless of the location of the effect; more particularly, the topical administration of the medicament comprises applying the medicament to a mucous membrane or lining of the gastrointestinal tract of a subject, including, but not limited to, a mucous membrane or lining containing one or more disease sites, such as gastrointestinal mucosal lesions. The effect of the topical delivery or topical administration of the medicament may be local to, or away from, the site of the topical administration. “Topical delivery,” “topical administration,” “topical application” and “topical treatment” are used interchangeably herein.

In some exemplary embodiments, the method of delivery can include a delivery of the aqueous formulation to a section or subsection of the GI tract of a subject containing the one or more disease sites. The one or more disease sites can include stomach, duodenum, jejunum, ileum, terminal ileum, cecum, colon, ascending colon, transverse colon, descending colon, sigmoid colon or rectum.

In some exemplary embodiments, the method of delivery can include a delivery of the aqueous formulation to a portion of the GI tract.

As used herein, a “portion” of the GI tract refers to an anatomical section or subsection of the GI tract. Non-limiting examples of a portion of the GI tract include the mouth, the esophagus, the stomach, the duodenum, the jejunum, the ileum, the cecum, the colon, the ascending colon, the transverse colon, the descending colon, and the rectum.

In some exemplary embodiments, the aqueous formulation can be administered to (a) to a section or subsection of the gastrointestinal (GI) tract of the subject; or (b) proximal to a section or subsection of the gastrointestinal (GI) tract of the subject. In some exemplary embodiments, the section or subsection of the GI tract can contain one or more disease sites. In some specific exemplary embodiments, the one or more disease sites can be selected from the group consisting of stomach, duodenum, jejunum, ileum, cecum, ascending colon, transverse colon, descending colon, sigmoid colon and rectum; preferably, the section or subsection of the GI tract containing the one or more disease sites can be selected from the group consisting of ileum, cecum, colon and rectum; and a combination thereof.

As used herein, “proximate” as disclosed in connection with release of a drug from a device to one or more disease sites, refers to a location that can be sufficiently spatially close to the one or more disease sites such that releasing the drug at the location treats the disease. For example, when the drug can be released proximate to the one or more disease sites, the drug may be released about 150 cm or less, such as about 125 cm or less, such as about 100 cm or less, such as about 50 cm or less, such as about 40 cm or less, such as about 30 cm or less, such as about 20 cm or less, such as about 10 cm or less, such as about 5 cm or less, such as about 2 cm or less, from the one or more sites of disease. In some exemplary embodiments, the proximate location for drug release can be the same section or subsection of the gastrointestinal tract containing the one or more disease site. Thus, where the present application refers to release of a drug proximate to a site of disease, this in some exemplary embodiments refers to release of the drug to a section or subsection of the GI tract that contains a site of disease. For example, when a disease site can be in the ileum, the drug may be released proximate to the disease site by releasing the drug to the ileum. In some exemplary embodiments, the proximate location for drug release can be a different section or subsection of the GI tract than that containing the disease site; for example, the drug release may be proximal to the disease site. Thus, where the present application refers to release of a drug proximate to a site of disease, this in some exemplary embodiments refers to release of the drug to a section or subsection of the GI tract that can be proximal to the section or subsection containing the disease site. For example, when a disease site can be in the colon, the drug may be released to the cecum.

In some exemplary embodiments, the method of delivery can include a delivery using a device selected from an endoscope, an ingestible device, or a reservoir.

In some exemplary embodiments, the method of delivery can include an endoscope capable of delivering the aqueous formulations described herein. In some exemplary embodiments, the endoscope can comprise a catheter. In some exemplary embodiments, the catheter can be a spray catheter. In some exemplary embodiments, the endoscope can be connected to the reservoir. In some exemplary embodiments, the reservoir can be an anchorable reservoir. As a non-limiting example, the Olypmus PW-205V can be a ready-to-use spray catheter that enables efficient spraying for maximal differentiation of tissue structures during endoscopy, but may also be used to deliver drugs diseased tissue. In a review of robotic endoscopic capsules, Journal of Micro-Bio Robotics 11.1-4 (2016):1-18, Ciuti et al. state that progress in micro-electromechanical systems (MEMS) technologies have led to the development of new endoscopic capsules with enhanced diagnostic capabilities, in addition to traditional visualization of mucosa (embedding, e.g. pressure, pH, blood detection and temperature sensors).

As used herein, the term “ingestible” can mean that the device can be swallowed whole.

In some exemplary embodiments, the method of delivery can include an ingestible device capable of delivering the aqueous formulations described herein. Examples of such ingestible device are described in U.S. Pat. Publication No. 2017/0173045, U. S. Pat. Publication No. 2018/0070857, U.S. Pat. Publication No. 2018/0279908, U. S. Pat. Publication No. 2018/0168489, U.S. Pat. Publication No. 2018/0070857 and U.S. Pat. Publication No. 2018/0168490, each of which are being incorporated by reference in their entirety.

In another exemplary embodiment, the ingestible device can comprise a housing, a reservoir containing the aqueous formulation, and a release mechanism for releasing the aqueous formulation from the device, wherein the reservoir can be internal to the device.

As used herein, a “housing” can be a portion of an ingestible device that defines the boundary between the interior of the device and the environment exterior to the device.

In some exemplary embodiments, the ingestible device can comprise a housing, a reservoir containing the aqueous formulation, and a release mechanism for releasing the aqueous formulation from the device, wherein the reservoir can be releasably or permanently attached to the exterior of the housing or internal to the housing.

In another exemplary embodiment, the ingestible device can comprise a housing, a reservoir containing the aqueous formulation and a release mechanism for releasing the aqueous formulation from the device; wherein the reservoir can be releasably or permanently attached to the exterior of the housing or internal to the housing and the ingestible device releases the aqueous formulation at a location in the gastrointestinal tract of the subject that can be proximate to one or more sites of disease.

In some exemplary embodiments, the housing can be non-biodegradable in the GI tract.

In some exemplary embodiments, the reservoir can be made of a material that allows the formulation to leave the reservoir. In some specific exemplary embodiments, the material can be a biodegradable material.

In some exemplary embodiments, the release of the formulation can be triggered autonomously.

As used herein, an “autonomous device” refers to a device comprising one or more processors configured to independently control certain mechanisms or operations of the device while in the GI tract of a subject. Preferably, an autonomous device of the present disclosure has no external electrical or wireless connections that control device mechanisms or operations, although connections such as wireless connections may be present to enable alternative device functions, such as transmitting data collected by the device to an external (ex vivo) system or receiver. The independently controlled mechanisms or operations of the autonomous device include, for example, triggering the release of a drug (or the formulation comprising the drug), and/or determining the location of the device within the GI tract of the subject. Such mechanisms are referred to herein as “autonomous mechanisms,” or, for example, an “autonomous triggering mechanism” or an “autonomous localization mechanism,” respectively. Actively implementing such an autonomous triggering or autonomous localization mechanism can be referred to as “autonomous triggering” or “autonomous localizing,” respectively. An “autonomous localization mechanism” can be synonymous with a “self-localization mechanism.”

In some exemplary embodiments, the device can be programmed to release the formulation with one or more release profiles that may be the same or different at one or more locations in the GI tract. In some specific exemplary embodiments, the one or more locations can be predetermined.

In some exemplary embodiments, the device can be programmed to release the formulation at a location proximate to one or more sites of disease. In some exemplary embodiments, the release of the formulation can be triggered using a pH and/or bacteria-sensitive material.

In some exemplary embodiments, the release of the formulation can be triggered by a pre-programmed algorithm.

In some exemplary embodiments, the release of the formulation can be triggered by data from a sensor or detector to identify the location of the device.

In some exemplary embodiments, the device can comprise a detector configured to detect light reflectance from an environment external to the housing.

In some exemplary embodiments, the release can be triggered autonomously or based on the detected reflectance.

In some exemplary embodiments, the release mechanism can be an actuation system.

In some exemplary embodiments, the release mechanism can be a chemical actuation system.

In some exemplary embodiments, the release mechanism can be a mechanical actuation system.

In some exemplary embodiments, the release mechanism can be an electrical actuation system.

In some exemplary embodiments, the actuation system can comprise a pump.

In some exemplary embodiments, releasing the formulation can comprises pumping the formulation out of the reservoir.

In some further exemplary embodiments, the actuation system can comprise a gas generating cell.

In some exemplary embodiments, the device can further comprise an anchoring mechanism.

In some exemplary embodiments, the device can further include a self-localization mechanism configured to determine the device location within the subject's GI tract.

In some exemplary embodiments, the device can further comprise one more machine-readable hardware storage devices storing instructions that are executable by the one or more processing devices to (a) determine a location of the ingestible device in the GI tract of the subject; and (b) release the formulation from the device at a pre-selected location of the GI tract. In certain exemplary embodiments, the one or more machine readable hardware storage devices can store instructions that are executable by the one or more processing devices to transmit data to an external device (e.g., a base station external to the subject, such as a base station carried on an article worn by the subject) capable of implementing the data to determine the location of the device within the GI tract of the subject.

At least some of the elements of the various embodiments of the ingestible device described herein that can be implemented via software may be written in a high-level procedural language such as object oriented programming, a scripting language or both. Accordingly, the program code may be written in C, C⁺⁺ or any other suitable programming language and may comprise modules or classes, as is known to those skilled in object oriented programming. Alternatively, or in addition, at least some of the elements of the embodiments of the ingestible device described herein that are implemented via software may be written in assembly language, machine language or firmware as needed. In either case, the language may be a compiled or an interpreted language.

At least some of the program code used to implement the ingestible device can be stored on a storage media or on a computer readable medium that is readable by a general or special purpose programmable computing device having a processor, an operating system and the associated hardware and software that is necessary to implement the functionality of at least one of the embodiments described herein. The program code, when read by the computing device, configures the computing device to operate in a new, specific and predefined manner in order to perform at least one of the methods described herein.

Furthermore, at least some of the programs associated with the systems, devices, and methods of the example embodiments described herein are capable of being distributed in a computer program product comprising a computer readable medium that bears computer usable instructions for one or more processors. The medium may be provided in various forms, including non-transitory forms such as, but not limited to, one or more diskettes, compact disks, tapes, chips, and magnetic and electronic storage. In some exemplary embodiments, the medium may be transitory in nature such as, but not limited to, wire-line transmissions, satellite transmissions, internet transmissions (e.g., downloads), media, digital and analog signals, and the like. The computer useable instructions may also be in various formats, including compiled and non-compiled code.

The techniques described herein can be implemented using software for execution on a computer. For instance, the software forms procedures in one or more computer programs that execute on one or more programmed or programmable computer systems (which may be of various architectures such as distributed, client/server, or grid) each including at least one processor, at least one data storage system (including volatile and non-volatile memory and/or storage elements), at least one input device or port, and at least one output device or port.

The software may be provided on a storage medium, such as a CD-ROM, readable by a general or special purpose programmable computer or delivered (encoded in a propagated signal) over a communication medium of a network to the computer where it is executed. All of the functions may be performed on a special purpose computer, or using special-purpose hardware, such as coprocessors. The software may be implemented in a distributed manner in which different parts of the computation specified by the software are performed by different computers. Each such computer program is preferably stored on or downloaded to a storage media or device (e.g., solid state memory or media, or magnetic or optical media) readable by a general or special purpose programmable computer, for configuring and operating the computer when the storage media or device is read by the computer system to perform the procedures described herein. The inventive system may also be considered to be implemented as a computer-readable storage medium, configured with a computer program, where the storage medium so configured causes a computer system to operate in a specific and predefined manner to perform the functions described herein.

In some exemplary embodiments, the device can further comprise a force generator that generates a force, thereby initiating a release of the formulation from the ingestible device into a pre-selected location of the GI tract. In some specific exemplary embodiments, the force generator can be a gas generating cell that generates a gas.

In some exemplary embodiments, the device can further include a method for determining the device location within the subject's GI tract via a device self-localization mechanism. In some exemplary embodiments, determining the device location within the subject's GI tract via the device self-localization mechanism includes detecting one or more device transitions between portions of the subject's GI tract. In some exemplary embodiments, the one or more detected device transitions occurs between portions of the GI tract selected from the group consisting of: mouth and stomach; esophagus and stomach; stomach and duodenum; duodenum and jejunum; jejunum and ileum; ileum and cecum; and cecum and colon; and combinations of any two or more of the foregoing. In some exemplary embodiments, the portions are adjacent portions. In some exemplary embodiments, determining the device location within the subject's GI tract via the device self-localization mechanism includes confirming the one or more device transitions between the portions of the GI tract of the subject.

In some exemplary embodiments, the device self-localization mechanism can be based on data comprising light reflectance occurring external to the device and within the GI tract of the subject. In some exemplary embodiments, the device self-localization mechanism can be based on data comprising elapsed time after entry of the device into the GI tract of the subject, elapsed time after detecting at least one of the one or more device transitions between the portions of the subject's GI tract, or a combination thereof. In some exemplary embodiments, the device self-localizes to the stomach, duodenum, jejunum, ileum, cecum or colon with at least 80% accuracy. In some exemplary embodiments, the device self-localizes to the stomach, duodenum, jejunum, ileum, cecum or colon with at least about 85% accuracy.

As used herein, “accuracy,” when disclosed in connection with a specified location of a device within the GI tract of a subject, refers to the degree to which the location determined by the device conforms to the correct location, wherein the correct location can be based on a generally accepted standard. The location within the GI tract of the subject determined by the device can be based on data, for example, light reflectance data, collected by the ingestible device. In some exemplary embodiments, the correct location can be based on external imaging devices, such as computer-aided tomography (CT), interpreted, for example, by a qualified clinician or physician. Therefore, percent accuracy (“% accuracy”) can refer to the percentage agreement between the location of the device in the GI tract as determined by the device, and the correct location, for example, as determined by CT, e.g., expressed as [(number of devices in which location determined by the device agrees with location as determined by CT/total devices administered to the subject or subjects)×100%], or, where only one device can be administered per subject, [(number of subjects in which location determined by the device agrees with location as determined by CT/total number of subjects)×100%]. In some exemplary embodiments, the accuracy with which the device determines a location refers to the accuracy with which the device determines that it can be at a location pre-selected for drug release.

In some exemplary embodiments, the release of the formulation from the device can be autonomously triggered based on the self-localization of the device to a pre-selected location within the subject's GI tract. In some exemplary embodiments, the pre-selected location can be selected from the group consisting of the stomach, the duodenum, the jejunum, the ileum, the cecum and the colon. In some exemplary embodiments, the release of the formulation from the device occurs at substantially the same time as the device self-localizes to the pre-selected location. In some exemplary embodiments, the release of the formulation from the device commences within a period of time of at most about 5 minutes after the device detects or confirms the transition to the pre-selected location. In some exemplary embodiments, the period of time can be at most about 1 minute, at most about 30 seconds, at most about 10 seconds, or at most about 1 second after the device detects or confirms the transition to the pre-selected location. In some exemplary embodiments, the release of the formulation can be as a bolus.

In some exemplary embodiments, the release of the formulation from the device occurs over a pre-determined period of time. In some exemplary embodiments, the pre-determined period of time over which the formulation can be released from the device can be about 8 hours, about 7 hours, about 6 hours, about 5 hours, about 4 hours, about 3 hours, about 2 hours, about 1 hour, about 30 minutes, about 15 minutes, about 10 minutes, or about 5 minutes. In some exemplary embodiments, the pre-determined period of time commences within at most about 5 minutes, at most about 1 minute, at most about 30 seconds, at most about 10 seconds, or at most about 1 second after the device detects or confirms the transition to the pre-selected location.

In some exemplary embodiments, the release of the formulation from the device can be proximal to the section or subsection of the GI tract containing the one or more disease sites. In some exemplary embodiments, the release of the formulation can be to a section or subsection of the GI tract immediately proximal to (immediately preceding) the section or subsection of the subject's GI tract containing at least one of the one or more disease sites. In some exemplary embodiments, the immediately preceding section or subsection of the GI tract does not contain a disease site and/or has not been determined to contain a disease site.

In some exemplary embodiments, the section of the GI tract containing the one or more inflammatory disease sites can be selected from the group consisting of the stomach, duodenum, jejunum, ileum, cecum, ascending colon, transverse colon, descending colon, sigmoid colon and rectum; and a combination of any two or more of the foregoing. In some embodiment, the subsection of the GI tract containing the one or more inflammatory disease sites can be selected from the group consisting of the proximal duodenum, distal duodenum, proximal jejunum, distal jejunum, proximal ileum, distal ileum, proximal cecum, distal cecum, proximal ascending colon, distal ascending colon, proximal transverse colon, distal transverse colon, proximal descending colon and distal descending colon, and a combination of any two or more of the foregoing.

In some exemplary embodiments, the method of delivery can include a self-localizing device capable of delivering the aqueous formulation.

As used herein, a “self-localizing device” can refer to a device comprising a mechanism or system that can be implemented autonomously to determine the location of the ingestible device in vivo, e.g., within the GI tract of a subject. Such a mechanism can be referred to as a “self-localization mechanism.” A “self-localization mechanism” can be synonymous with an “autonomous localization mechanism.” A self-localizing device does not require ex vivo visualization devices or systems, for example, using scintigraphy or computer-aided tomography (CT), to localize in the GI tract.

In some exemplary embodiments of the method, the device self-localization mechanism does not require monitoring the pH of the subject's GI tract. In some exemplary embodiments, the method excludes a pH-dependent drug release mechanism. In some exemplary embodiments, the device self-localization mechanism does not require monitoring the pressure of the subject's GI tract, the temperature of the subject's GI tract, or both.

In some exemplary embodiments, the method of delivery can include localizing a device comprising the aqueous formulation. In some exemplary embodiments, the method of delivery can include localizing a device comprising the aqueous formulation and releasing the aqueous formulation.

As used herein, “localizing a device” can refer to determining a location of the device.

As used herein, “self-localizing a device” can refer to determining a location of the device via a device self-localization mechanism, e.g., determining a location of the within the GI tract of a subject via a device self-localization mechanism.

In other embodiments, the method of delivery can include a rectal administration. In some specific exemplary embodiments, the aqueous formulation can be an enema for rectal administration.

Various publications, including patents, patent applications, published patent applications, accession numbers, technical articles and scholarly articles are cited throughout the specification. Each of these cited references is incorporated by reference, in its entirety and for all purposes, herein.

The disclosure will be more fully understood by reference to the following Examples, which are provided to describe the disclosure in greater detail. They are intended to illustrate and should not be construed as limiting the scope of the disclosure.

### NON-LIMITING EMBODIMENTS

The following Embodiments are detailed by way of illustration only and are not to be construed as limiting in spirit or in scope, many modifications both in materials and in methods will be apparent to those skilled in the art.
1. An aqueous formulation comprising:
   tofacitinib or a pharmaceutically acceptable salt thereof; and cyclodextrin at a concentration of at least about 10% w/w;
   wherein, after extended period of time under storage condition, at least about 75% of the tofacitinib or a pharmaceutically acceptable salt thereof remains in the aqueous formulation; or
   wherein the tofacitinib or a pharmaceutically acceptable salt thereof is present in the aqueous formulation at a concentration of at least about 5 mg/mL.
2. The aqueous formulation of embodiment 1, wherein at least about 80% of the tofacitinib or a pharmaceutically acceptable salt thereof remains in the aqueous formulation after extended period of time under storage condition.
3. The aqueous formulation of embodiment 1, wherein at least about 85% of the tofacitinib or a pharmaceutically acceptable salt thereof remains in the aqueous formulation after extended period of time under storage condition.
4. The aqueous formulation of embodiment 1, wherein at least about 90% of the tofacitinib or a pharmaceutically acceptable salt thereof remains in the aqueous formulation after extended period of time under storage condition.
5. The aqueous formulation of embodiment 1, wherein at least about 93% of the tofacitinib or a pharmaceutically acceptable salt thereof remains in the aqueous formulation after extended period of time under storage condition.
6. The aqueous formulation of embodiment 1, wherein at least about 95% of the tofacitinib or a pharmaceutically acceptable salt thereof remains in the aqueous formulation after extended period of time under storage condition.
7. The aqueous formulation of embodiment 1, wherein at least about 97% of the tofacitinib or a pharmaceutically acceptable salt thereof remains in the aqueous formulation after extended period of time under storage condition.
8. The aqueous formulation of embodiment 1, wherein at least about 98% of the tofacitinib or a pharmaceutically acceptable salt thereof remains in the aqueous formulation after extended period of time under storage condition.
9. The aqueous formulation of embodiment 1, wherein at least about 99% of the tofacitinib or a pharmaceutically acceptable salt thereof remains in the aqueous formulation after extended period of time under storage condition.
10. The aqueous formulation of any one of the embodiments 1-9, wherein the extended period of time is about 1 week.
11. The aqueous formulation of any one of the embodiments 1-9, wherein the extended period of time is about 2 weeks.
12. The aqueous formulation of any one of the embodiments 1-9, wherein the extended period of time is about 3 weeks.
13. The aqueous formulation of any one of the embodiments 1-9, wherein the extended period of time is about 1 month.
14. The aqueous formulation of any one of the embodiments 1-9, wherein the extended period of time is about 2 months.
15. The aqueous formulation of any one of the embodiments 1-9, wherein the extended period of time is about 3 months.
16. The aqueous formulation of any one of the embodiments 1-9, wherein the extended period of time is about 4 months.
17. The aqueous formulation of any one of the embodiments 1-9, wherein the extended period of time is about 5 months.
18. The aqueous formulation of any one of the embodiments 1-9, wherein the extended period of time is about 6 months.
19. The aqueous formulation of any one of the embodiments 1-9, wherein the extended period of time is about 8 months.
20. The aqueous formulation of any one of the embodiments 1-9, wherein the extended period of time is about 10 months.
21. The aqueous formulation of any one of the embodiments 1-9, wherein the extended period of time is about 12 months.
22. The aqueous formulation of any one of the embodiments 1-9, wherein the extended period of time is about 15 months.
23. The aqueous formulation of any one of the embodiments 1-9, wherein the extended period of time is about 18 months.
24. The aqueous formulation of any one of the embodiments 1-9, wherein the extended period of time is about 21 months.
25. The aqueous formulation of any one of the embodiments 1-9, wherein the extended period of time is about 24 months.
26. The aqueous formulation of any one of the embodiments 1-25, wherein the storage condition has a storage temperature of about 2-8° C.
27. The aqueous formulation of any one of the embodiments 1-25, wherein the storage condition has a storage temperature of about 5° C.
28. The aqueous formulation of any one of the embodiments 1-25, wherein the storage condition has a storage temperature of about 25° C.
29. The aqueous formulation of any one of the embodiments 1-25, wherein the storage condition has a storage temperature of about 25° C. and a relative humidity of about 65%.
30. The aqueous formulation of any one of the embodiments 1-25, wherein the storage condition has a storage temperature of about 40° C. and a relative humidity of about 75%.
31. The aqueous formulation of any one of the embodiments 1-30, wherein the tofacitinib salt form is an acid addition salt.
32. The aqueous formulation of any one of the embodiments 1-31, wherein the tofacitinib salt is selected from the group consisting of tofacitinib aceglutamate, acephyllinate, acetamidobenzoate, 2-acetamidobenzoate, 4-acetamidobenzoate, acetate, acetylasparaginate, acetylaspartate, acid citrate, adipate, acetylsalicylate, aminosalicylate, anhydromethylenecitrate, ascorbate, aspartate, benzenesulfonate (besylate), benzoate, bicarbonate, bisulfate, bitartrate, borate, bromide, butanoate, caffeate, caftarate, camphorate (+-camphorate), camsylate (camsilate, camphorsulfonic acid), carbonate, cholate, chloride, chlorogenate, chlorophenoxyacetate, cis-cinnamate, trans-cinnamate, citrate, closylate (4-chlorobenzene sulfonate), cromesilate (6,7-Dihydroxycourmarin-4-methanesulfonate), cyclamate, cyclohexanecarboxylate, decanoate (caprate), dehydrocholate, 2,5-dihydroxybenzoate (gentisate), edetate (EDTA), edisylate (1,2-ethanedisulfonate), erythorbate (isoascorbate), estolate (laurylsulfate), esylate (ethanesulfonate), ethylsulfate, fendizoate, ferulate, formate, fumarate, gluceptate, glucoheptanoate (2,3,4,5,6,7-hexahydroxyheptanoic acid), gluconate (D-gluconate), glucuronate, glutamate, glutarate, glycerophosphate, glycinate, glycolate, glycyrrhizate, glyoxylate (oxaldehydate), hexanoate, hippurate, hydrobromide, hydrochloride, hydroiodide, 4-hydroxybenzenesulfonate, 3-hydroxybenzoate, 4-hydroxybenzoate, hydroxynaphthoate, 3-hydroxybutanoate, 4-hydroxybutanoate, 4-hydroxycinnamate (p-coumarate), 3-hydroxypropionate, iodide, isethionate (2-hydroxyethanesulfonate), isopropyl mesylate (isopropyl methanesulfonate), lactate (D,L-lactate), lactobionate (erythromycin mono (4-0-β-D-galactopyranosyl-D-gluconate)), laurate, lipoate ((R)-lipoate), lysine, malate (2-hydroxybutanedioic acid), maleate ((Z)-but-2-enedioate), mandelate (2-hydroxy-2-phenylacetate), mesotartarate, mesylate (methanesulfonate), metaphosphate, 4-methylpentanoate, 2-methylpropionate, methylsuccinate, methylsulfate, mucate (galactarate; (2S,3R,4S,5R)-2,3,4,5-tetrahydroxyhexanedioate), napadisylate (1,5-naphthalenedisulfonate), napsylate (naphthalene-2-sulfonate), nicotinate, nitrate, octanoate (caprylate), oleate, orotate, oxalate (ethanedioate), oxoglurate, palmitate, pamoate (3-carboxy-1-[(3-carboxy-2-oxidonaphthalen-1-yl)methyl]naphthalen-2-olate), pantothenate (Vitamin B5), pectinate, pentanoate, phenylacetate, phenylethylbarbiturate, phosphate, picrate, pimelate (heptanedioate), polygalacturonate (pectate), polyglutamate, propionate (propanoate), pyridoxal phosphate, L-pyroglutamate, pyruvate, quinate, saccharate, saccharinate, salicylate, sebacate, sinapate, sorbate, stearate, stearylsulfate, succinate, sulfate, sulfosalicylate, tannate, tartrate, teoclate, terephthalate, thiocyanate, tosylate (toluene 4-sulfonate), trans-coutarate, trimethylacetate, undecanoate (undecylate), urate and xinafoate (1-hydroxy-2-naphthoate).
33. The aqueous formulation of any one of the embodiments 1-31, wherein the tofacitinib salt is selected from the group consisting of tofacitinib aceglutamate, acephyllinate, acetamidobenzoate, 2-acetamidobenzoate, 4-acetamidobenzoate, acetate, acetylasparaginate, acetylaspartate, acid citrate, adipate, acetylsalicylate, aminosalicylate, anhydromethylenecitrate, aspartate, benzoate, bicarbonate, bitartrate, butanoate, caffeate, caftarate, camphorate, carbonate, chlorogenate, cis-cinnamate, trans-cinnamate, citrate, cholate, chlorophenoxyacetate, cyclohexanecarboxylate, decanoate (caprate), dehydrocholate, 2,5-dihydroxybenzoate (gentisate), edetate (EDTA), fendizoate, ferulate, gluceptate, glucoheptanoate (2,3,4,5,6,7-hexahydroxyheptanoic acid), gluconate (D-gluconate), glucuronate, glutamate, glutarate, glycinate, glycolate, glycyrrhizate, glyoxylate (oxaldehydate), hexanoate, hippurate, 3-hydroxybenzoate, 4-hydroxybenzoate, 3-hydroxybutanoate, 4-hydroxybutanoate, 4-hydroxycinnamate (p-coumarate), 3-hydroxypropionate, hydroxynaphthoate, lactate, lactobionate (erythromycin mono (4-0-β-D-galactopyranosyl-D-gluconate), laurate, lipoate ((R)-lipoate), lysine, malate (2-hydroxybutanedioic acid), maleate ((Z)-but-2-enedioate), mandelate (2-hydroxy-2-phenylacetate), mesotartarate, 4-methylpentanoate, 2-methylpropionate, methylsuccinate, mucate (galactarate; (2S,3R,4S,5R)-2,3,4,5-tetrahydroxyhexanedioate), nicotinate, octanoate (caprylate), oleate, orotate, oxalate (ethanedioate), oxoglurate, palmitate, pamoate (3-carboxy-1-[(3-carboxy-2-oxidonaphthalen-1-yl)methyl]naphthalen-2-olate), pantothenate (Vitamin B5), pectinate, pentanoate, phenylacetate, pimelate (heptanedioate), polygalacturonate (pectate), polyglutamate, propionate (propanoate), L-pyroglutamate, pyruvate, quinate, saccharate, salicylate, sebacate, sinapate, sorbate, stearate, succinate, sulfosalicylate, tartrate, terephthalate, trans-coutarate, trimethylacetate, undecanoate (undecylate) and xinafoate (1-hydroxy-2-naphthoate).
34. The aqueous formulation of any one of the embodiments 1-31, wherein the pharmaceutically acceptable salt is selected from the group consisting of benzenesulfonate (besylate), bisulfate, camsylate, closylate (4-chlorobenzene sulfonate), cromesilate (6,7-dihydroxycourmarin-4-methanesulfonate), cyclamate, edisylate (1,2-ethanedisulfonate), estolate (laurylsulfate), esylate (ethanesulfonate), ethylsulfate, 4-hydroxybenzenesulfonate, isethionate (2-hydroxyethanesulfonate), isopropyl mesylate (isopropyl methanesulfonate), mesylate, methylsulfate, napadisylate (1,5-naphthalenedisulfonate), napsylate (naphthalene-2-sulfonate), stearylsulfate, sulfate, sulfosalicylate and tosylate (p-toluenesulfonate).
35. The aqueous formulation of any one of the embodiments 1-31, wherein the tofacitinib salt is selected from the group consisting of tofacitinib chloride, bromide and iodide.
36. The aqueous formulation of any one of the embodiments 1-31, wherein the tofacitinib salt is selected from the group consisting of tofacitinib ascorbate, borate, glycerophosphate, erythorbate (isoascorbate), metaphosphate, nitrate, phenylethylbarbiturate, phosphate, picrate, pyridoxal phosphate, saccharinate, tannate, teoclate and thiocyanate.
37. The aqueous formulation of any one of the embodiments 1-31, wherein the tofacitinib salt is selected from the group consisting of tofacitinib citrate, oxalate, malate, tartrate and acetate.
38. The aqueous formulation of any one of the embodiments 1-31, wherein the tofacitinib salt is tofacitinib citrate.
39. The aqueous formulation of embodiment 38, wherein the tofacitinib citrate is tofacitinib monocitrate.
40. The aqueous formulation of any one of the embodiments 1-39, further comprising a stabilizer.
41. The aqueous formulation of embodiment 40, wherein the stabilizer is tromethamine (TRIS).
42. The aqueous formulation of embodiment 40, wherein the stabilizer is glycine.
43. The aqueous formulation of embodiment 40, wherein the stabilizer is a polymer.
44. The aqueous formulation of embodiment 43, wherein the polymer is a polyacrylic acid polymer.
45. The aqueous formulation of embodiment 44, wherein the polyacrylic acid polymer is a crosslinked polyacrylic acid polymer.
46. The aqueous formulation of embodiment 45, wherein the crosslinked polyacrylic acid polymer is a Carbomer®.
47. The aqueous formulation of any one of the embodiments 44-46, wherein the polyacrylic acid polymer is a Carbopol®.
48. The aqueous formulation of any one of the embodiments 44-46, wherein the polyacrylic acid polymer is a homopolymer.
49. The aqueous formulation of embodiment 48, wherein the polyacrylic acid homopolymer comprises an acrylic acid crosslinked with allyl sucrose or allyl pentaerythritol.
50. The aqueous formulation of embodiment 48 or 49, wherein the polyacrylic acid homopolymer is selected from a group consisting of Carbomer homopolymer type A, Carbomer homopolymer type B and Carbomer homopolymer type C; and a combination of any two or more of the foregoing.
51. The aqueous formulation of embodiment 50, wherein the Carbomer homopolymer type A is Carbopol® 71G, Carbopol® 971P and Carbopol® 981.
52. The aqueous formulation of embodiment 50, wherein the Carbomer homopolymer type B is Carbopol® 974P and Carbopol® 5984.
53. The aqueous formulation of embodiment 50, wherein the Carbomer homopolymer type C is Carbopol® 980.
54. The aqueous formulation of embodiment 50, wherein the Carbopol® homopolymer is Carbopol® 971P.
55. The aqueous formulation of embodiment 44, wherein the polyacrylic acid polymer is a Carbomer copolymer.
56. The aqueous formulation of embodiment 55, wherein the Carbomer copolymer comprises an acrylic acid and a C₁₀-C₃₀ alkyl acrylate crosslinked with allyl pentaerythritol.
57. The aqueous formulation of embodiment 55 or 56, wherein the Carbomer copolymer is Carbopol® 1342, Pemulen TR-2 NF polymer or Pemulen TR-1 NF polymer.
58. The aqueous formulation of embodiment 44, wherein the polyacrylic acid polymer is a Carbomer interpolymer.
59. The aqueous formulation of embodiment 58, wherein the Carbomer interpolymer comprises a Carbomer homopolymer or copolymer containing a block copolymer of polyethylene glycol and a long chain alkyl acid ester.
60. The aqueous formulation of embodiment 58 or 59, wherein the Carbomer interpolymer is Carbopol® ETC 2020, Carbopol® Ultrez 10 or a combination thereof.
61. The aqueous formulation of any one of the embodiments 40-60, wherein the stabilizer is at a concentration ranging from about 0.05% w/w to about 3.0% w/w, from about 0.1% w/w to about 2.5% w/w, from about 0.25% w/w to about 2.0% w/w, or from about 0.5% w/w to about 1.5% w/w.
62. The aqueous formulation of embodiment 40, wherein the stabilize is TRIS, and wherein the TRIS: tofacitinib molar ratio is 3.0:1 to 2.0:1.
63. The aqueous formulation of embodiment 40, wherein the stabilize is TRIS, and wherein the TRIS: tofacitinib molar ratio is 3.0:1 to 2.5:1.
64. The aqueous formulation of embodiment 40, wherein the stabilize is TRIS, and wherein the TRIS: tofacitinib molar ratio is about 2.75:1.
65. The aqueous formulation of any one of the embodiments 1-64, wherein the cyclodextrin concentration is at least about 10%, about 15%, about 20%, about 25%, about 30%, about 35%, about 40%, about 45%, about 50%, about 55% about 60%, about 65%, about 70%, about 75%, about 80%, about 85%, or about 90%.
66. The aqueous formulation of any one of the embodiments 1-65, wherein the cyclodextrin is α-cyclodextrin, β-cyclodextrin or γ-cyclodextrin; or a derivative thereof.
67. The aqueous formulation of any one of the embodiments 1-65, wherein the cyclodextrin is a water soluble cyclodextrin.
68. The aqueous formulation of any one of the embodiments 1-65, wherein the cyclodextrin is selected from the group consisting of hydroxypropyl-α-cyclodextrin, hydroxypropyl-β-cyclodextrin, hydroxypropyl-γ-cyclodextrin, sulfobutylether β-cyclodextrin and methyl-β-cyclodextrin; or a combination of two or more of the foregoing.
69. The aqueous formulation of any one of the embodiments 1-65, wherein the cyclodextrin is β-cyclodextrin; or a derivative thereof.
70. The aqueous formulation of embodiment 69, wherein the β-cyclodextrin or derivative thereof is hydroxypropyl-β-cyclodextrin, sulfobutylether β-cyclodextrin or methyl-beta-cyclodextrin.
71. The aqueous formulation of any one of the embodiments 1-65, wherein the cyclodextrin is hydroxypropyl-β-cyclodextrin.
72. The aqueous formulation of embodiment 71, wherein the hydroxypropyl-β-cyclodextrin is present at a concentration ranging from about 60% to about 65% (w/w).
73. The aqueous formulation of any one of the embodiments 1-65, wherein the cyclodextrin is sulfobutylether-β-cyclodextrin.
74. The aqueous formulation of embodiment 73, wherein the sulfobutylether-β-cyclodextrin is present at a concentration ranging from about 10% to about 40% (w/w).
75. The aqueous formulation of any one of the embodiments 1-65, wherein the cyclodextrin is methyl-β-cyclodextrin.
76. The aqueous formulation of embodiment 75, wherein the methyl-β-cyclodextrin is present at a concentration ranging from about 35% to about 70% (w/w).
77. The aqueous formulation of any one of the embodiments 1-76, wherein pH of the aqueous formulation ranges from about pH 4.0 to about pH 8.
78. The aqueous formulation of any one of the embodiments 1-76, wherein pH of the aqueous formulation is from about pH 6 to about pH 8.
79. The aqueous formulation of any one of the embodiments 1-76, wherein pH of the aqueous formulation is from about pH 6 to about pH 7.
80. The aqueous formulation of any one of the embodiments 1-76, wherein pH of the aqueous formulation is about pH 6.5.
81. The aqueous formulation of any one of the embodiments 1-80, wherein at least about 95% of the aqueous formulation is a solution, a nano-suspension or a combination thereof.
82. The aqueous formulation of any one of the embodiments 1-81, wherein at most about 5% of the aqueous formulation is a micro-suspension.
83. The aqueous formulation of any one of the embodiments 1-82, wherein at least about 90% of the aqueous formulation passes through a 0.2 micron filter.
84. The aqueous formulation of any one of the embodiments 1-83 further comprising a pharmaceutically acceptable excipient.
85. The aqueous formulation of any one of the embodiments 1-84, wherein the viscosity of the aqueous formulation is less than about 4000 cP.
86. The aqueous formulation of any one of the embodiments 1-85, wherein the tofacitinib or a pharmaceutically acceptable salt thereof is present in the aqueous formulation at a concentration of at least about 5 mg/mL, at least about 10 mg/mL, at least about 15 mg/mL, at least about 20 mg/mL, at least about 25 mg/mL, at least about 30 mg/mL, at least about 35 mg/mL, at least about 40 mg/mL, at least about 45 mg/mL, at least about 50 mg/mL, at least about 55 mg/mL, at least about 60 mg/mL, at least about 65 mg/mL, at least about 65 mg/mL, at least about 70 mg/mL, at least about 75 mg/mL, at least about 80 mg/mL, at least about 85 mg/mL, at least about 90 mg/mL, at least about 95 mg/mL, or at least about 100 mg/mL.
87. The aqueous formulation of any one of the embodiments 1-85, wherein the tofacitinib or a pharmaceutically acceptable salt thereof is present in the aqueous formulation at a concentration of from about 10 mg/mL to about 140 mg/mL.
88. The aqueous formulation of any one of the embodiments 1-85, wherein the tofacitinib or a pharmaceutically acceptable salt thereof is present in the aqueous formulation at a concentration of from about 10 mg/mL to about 30 mg/mL.
89. The aqueous formulation of any one of the embodiments 1-85, wherein the tofacitinib or a pharmaceutically acceptable salt thereof is present in the aqueous formulation at a concentration of from about 30 mg/mL to about 50 mg/mL.
90. The aqueous formulation of any one of the embodiments 1-85, wherein the tofacitinib or a pharmaceutically acceptable salt thereof is present in the aqueous formulation at a concentration of from about 50 mg/mL to about 100 mg/mL.
91. The aqueous formulation of any one of the embodiments 1-85, wherein the tofacitinib or a pharmaceutically acceptable salt thereof is present in the aqueous formulation at a concentration of from about 80 mg/mL to about 100 mg/mL.
92. An aqueous formulation comprising:
   tofacitinib citrate at a concentration of about 10 mg/mL to about 140 mg/mL;
   cyclodextrin at a concentration of about 40% w/w to about 80% w/w; and
   tromethamine (TRIS) at a molar ratio of 3.5:1 to 1.5:1 (TRIS: tofacitinib).
93. The aqueous formulation of embodiment 92, wherein the concentration of tofacitinib citrate is about 10 mg/mL to about 30 mg/mL.
94. The aqueous formulation of embodiment 92, wherein the concentration of tofacitinib citrate is about 30 mg/mL to about 50 mg/mL.
95. The aqueous formulation of embodiment 92, wherein the concentration of tofacitinib citrate is about 80 mg/mL to about 100 mg/mL.
96. The aqueous formulation of any one of the embodiments 92-95, wherein the concentration of cyclodextrin is about 50% w/w to about 70% w/w.
97. The aqueous formulation of any one of the embodiments 92-95, wherein the concentration of cyclodextrin is about 55% w/w to about 70% w/w.
98. The aqueous formulation of any one of the embodiments 92-95, wherein the concentration of cyclodextrin is about 60% w/w to about 65% w/w.
99. The aqueous formulation of any one of the embodiments 92-98, wherein the TRIS: tofacitinib molar ratio is 3.0:1 to 2.0:1.
100. The aqueous formulation of any one of the embodiments 92-98, wherein the TRIS: tofacitinib molar ratio is 3.0:1 to 2.5:1.
101. The aqueous formulation of any one of the embodiments 92-98, wherein the TRIS: tofacitinib molar ratio is about 2.75:1.
102. An aqueous formulation comprising:
   tofacitinib citrate at a concentration of about 10 mg/mL to about 140 mg/mL;
   cyclodextrin at a concentration of about 40% w/w to about 80% w/w; and
   wherein the pH of the aqueous formulation ranges from about pH 6 to about pH 8.
103. The aqueous formulation of embodiment 102, wherein the concentration of tofacitinib citrate is about 10 mg/mL to about 30 mg/mL.
104. The aqueous formulation of embodiment 102, wherein the concentration of tofacitinib citrate is about 30 mg/mL to about 50 mg/mL.
105. The aqueous formulation of embodiment 102, wherein the concentration of tofacitinib citrate is about 80 mg/mL to about 100 mg/mL.
106. The aqueous formulation of any one of the embodiments 102-105, wherein the concentration of cyclodextrin is about 50% w/w to about 70% w/w.
107. The aqueous formulation of any one of the embodiments 102-105, wherein the concentration of cyclodextrin is about 55% w/w to about 70% w/w.
108. The aqueous formulation of any one of the embodiments 102-105, wherein the concentration of cyclodextrin is about 60% w/w to about 65% w/w.
109. The aqueous formulation of any one of the embodiments 102-108, wherein the pH of the aqueous formulation ranges from about pH 6 to about pH 7.
110. The aqueous formulation of any one of the embodiments 102-108, wherein the pH of the aqueous formulation is about pH 6.5.
111. A method of delivering tofacitinib to a subject, the method comprising:
   topically administering to a subject in need thereof an aqueous formulation according to any one of the embodiments 1-110.
112. The method of embodiment 111, wherein the aqueous formulation is released at a location in the gastrointestinal tract of the subject that is proximate to one or more sites of the disease or the condition.
113. The method of embodiment 111, wherein the aqueous formulation is released proximate to one or more sites of the disease or the condition selected from a group consisting of mouth, stomach, esophagus, stomach; duodenum, jejunum, ileum, cecum and colon.
114. The method of any one of embodiments 111-113, wherein the aqueous formulation is administered using an ingestible device.
115. The method of embodiment 114, wherein the ingestible device comprises a housing, a reservoir containing the aqueous formulation, and a release mechanism for releasing the aqueous formulation from the device, wherein the reservoir is releasably or permanently attached to the exterior of the housing or internal to the housing.
116. The method of embodiment 114, wherein the ingestible device comprises a housing, a reservoir containing the aqueous formulation, and a release mechanism for releasing the aqueous formulation from the device, wherein the reservoir is internal to the device.
117. The method of embodiment 114, wherein release of the aqueous formulation is triggered autonomously.
118. The method of embodiment 114, wherein the ingestible device is programmed to release the aqueous formulation at a location proximate to one or more sites of the disease or the condition.
119. The method of embodiment 114, wherein the location of one or more sites of the disease or the condition is predetermined.
120. The method of embodiment 114, wherein the release of the aqueous formulation is triggered by a pre-programmed algorithm.
121. The method of embodiment 114, wherein the release of the aqueous formulation is triggered by data from a sensor or detector to identify the location of the device.
122. The method of embodiment 114, wherein the release is triggered based on a detected reflectance from an external environment.
123. The method of any one of embodiments 111-113 wherein the aqueous formulation is administered using an endoscope.
124. The method of any one of embodiments 111-113, wherein the aqueous formulation is administered using an endoscope configured to a spray catheter.
125. The method of any one of embodiments 111-113, the aqueous formulation is administered using an endoscope connected to a reservoir.
126. The method of embodiment 125, wherein the reservoir is an anchorable reservoir.
127. The method of any one of embodiments 111-113, wherein the aqueous formulation is an enema for rectal administration.
128. An ingestible device comprising the aqueous formulation of any one of embodiments 1-54, 79, 81-87, 89-93, 95-97, and 99-105.
129. A device comprising the aqueous formulation of any one of embodiments 1-110.
130. An ingestible device, comprising:
   a housing;
   a reservoir; and
   a release mechanism, wherein the reservoir comprises the aqueous formulation of any one of embodiments 1-110.
131. The ingestible device of embodiment 130, wherein the reservoir is releasably or permanently attached to the exterior of the housing.
132. The ingestible device of embodiment 130, wherein the reservoir is releasably or permanently attached to the internal to the housing.
133. The ingestible device of embodiment 130, wherein a self-localization mechanism is configured to determine a location of the device within a subject's GI tract.
134. The ingestible device of embodiment 130, wherein release of the aqueous formulation can be triggered autonomously.
135. The ingestible device of embodiment 130, wherein release of the aqueous formulation from the device is autonomously triggered based on self-localization of the device to a pre-selected location within a subject's GI tract.
136. The ingestible device of embodiment 135, wherein the pre-selected location is selected from the group consisting of stomach, duodenum, jejunum, ileum, cecum and colon.
137. The ingestible device of embodiment 130, wherein the device is programmed to release the aqueous formulation at a location proximate to one or more sites of the disease or the condition.
138. The ingestible device of embodiment 130, wherein release of the aqueous formulation can be triggered by a pre-programmed algorithm.
139. The ingestible device of embodiment 130, wherein release of the aqueous formulation is triggered by data from a sensor or detector to identify a location of the device.
140. The ingestible device of embodiment 130, wherein release of the aqueous formulation is triggered based on a detected reflectance from an external environment.
141. The ingestible device of embodiment 130, wherein the device further comprises one more machine-readable hardware storage devices storing instructions that are executable by the one or more processing devices to (a) determine a location of the ingestible device in the GI tract of the subject; and (b) release the formulation from the device at a pre-selected location of the GI tract.
142. The ingestible device of embodiment 130, wherein the ingestible device further comprises a force generator generates a force, thereby initiating a release of the formulation from the ingestible device into a pre-selected location of the GI tract.
143. The ingestible device of embodiment 142, wherein the force generator is a gas generating cell that generates a gas.
144. An aqueous formulation comprising:
   tofacitinib citrate at a concentration of about 10 mg/mL to about 130 mg/mL;
   hydroxypropyl-β-cyclodextrin at a concentration of about 10% w/w to about 55% w/w; and
   Carbopol® 971P at a concentration ranging from about 0.05% w/w to about 1.5% w/w,
   wherein the pH of the aqueous formulation ranges from about pH 4.0 to about pH 8.
145. The aqueous formulation of embodiment 144, wherein the concentration of tofacitinib is about 25 mg/mL to about 60 mg/mL.
146. The aqueous formulation of embodiment 144, wherein the pH of the aqueous formulation ranges from about pH 4 to about pH 7.
147. The aqueous formulation of embodiment 144, wherein the pH of the aqueous formulation ranges from about pH 4.5 to about pH 5.5.
148. The aqueous formulation of embodiment 144, wherein the concentration of hydroxypropyl-p-cyclodextrin is about 40% w/w to about 55% w/w.
149. An aqueous formulation comprising:
   tofacitinib citrate at a concentration of about 10 mg/mL to about 150 mg/mL;
   sulfobutylether β-cyclodextrin at a concentration of about 10% w/w to about 40% w/w; and
   Carbopol® 971 at a concentration ranging from about 0.05% w/w to about 1.5% w/w,
   wherein the pH of the aqueous formulation ranges from about pH 4.0 to about pH 8.
150. The aqueous formulation of embodiment 149, wherein the pH of the aqueous formulation ranges from about pH 4 to about pH 7.
151. The aqueous formulation of embodiment 149, wherein the pH of the aqueous formulation ranges from about pH 4.5 to about pH 5.5.
152. An aqueous formulation comprising:
   tofacitinib citrate at a concentration of about 20 mg/mL to about 140 mg/mL and
   methyl-β-cyclodextrin at a concentration of about 35% w/w to about 70% w/w,
   wherein the pH of the aqueous formulation ranges from about pH 4.0 to about pH 8.
153. The aqueous formulation of embodiment 152, wherein the concentration of tofacitinib is about 25 mg/mL to about 140 mg/mL.
154. The aqueous formulation of embodiment 152, wherein the pH of the aqueous formulation ranges from about pH 4 to about pH 7.
155. The aqueous formulation of embodiment 152, wherein the pH of the aqueous formulation ranges from about pH 4.5 to about pH 5.5.
156. The aqueous formulation of embodiment 152, wherein the pH is about 5.5.
157. The aqueous formulation of embodiment 152, wherein the concentration of methyl-p-cyclodextrin is about 40% w/w to about 70% w/w.
158. The aqueous formulation of embodiment 152, wherein the concentration of methyl-p-cyclodextrin is about 56% w/w.

### NON-LIMITING EXAMPLES

The following Examples are detailed by way of illustration only and are not to be construed as limiting in spirit or in scope, many modifications both in materials and in methods will be apparent to those skilled in the art.

### Material and Reagents.

Tofacitinib free base and tofacitinib citrate were purchased from Microlabs, India. Water was sourced from in-house 18 MQ Milli-Q Water. 10 N NaOH was purchased from Fischer Scientific. Triethylamine (TEA) was purchased from Sigma-Aldrich (St Louis, MO). 6 N HCl (aq) was purchased from EMD Millipore. The citrate buffer was made in-house using anhydrous citric acid and sodium citrate dehydrate. Alternatively, tris(hydroxymethyl)aminomethane or glycine can be used as buffers for use in the formulations described herein. HPβCD, HPαCD, HPγCD, MβCD and SBEβCD were purchased from Cyclolab, Roquette Pharma, Sigma Aldrich, or Acros. Carbopol 971P was purchased from Essential Ingredients. All reagents were used without additional purification.

pH measurement. pH of the formulation was adjusted using hydrochloric acid (1N or 6N) “--” indicates pH not adjusted or measured; “-- (value)” indicates pH was not adjusted, with measured value shown in parenthesis. The pH was measured using a standard laboratory pH meter at 25° C.

Viscosity measurement. The viscosity of an aqueous formulation corresponds to the dynamic viscosity of said aqueous formulation. The viscosity was measured at 25° C. with a Brookfield digital viscometer. The viscosity of an aqueous formulation is measured shortly after, i.e. less than 24 hours after, the preparation of the aqueous formulation.

Percentage of drug in solid complex and percentage of dissolved drug. The amount of drug in the form of solid complexes and the amount of dissolved drug is obtained by centrifuging the aqueous formulation at 6000 rpm at room temperature for 20-30 minutes.

The amount of dissolved drug corresponds to the amount of drug in the supernatant as measured by high-performance liquid chromatography. The percentage of drug in the form of a solid complex is obtained with the following formula:

$$\% drug in solid complex = \frac{( total drug - dissolved drug )}{total drug} \times 100 ,$$

wherein total drug is the total amount of drug introduced in the aqueous formulation in mg/mL; and dissolved drug is the amount of drug in the supernatant in mg/mL.

The percentage of dissolved drug is obtained with the following formula:

% dissolved drug=100−% drug in solid complex

### Example 1. Evaluation of Effect of Stabilizer on Drug Solubility

The aqueous formulations listed in Table 1 were prepared by the following methods:

Entry 1: Tofacitinib citrate (1.20 g) was added to 10 mL water. This mixture was stirred until all solids dissolved to afford a clear viscous liquid. The clear viscous liquid was filtered via 0.2 μm filter.

Entry 2: Tofacitinib citrate (1.20 g) was added to 10 mL 50 mM citrate buffer, pH 4.0. This mixture was stirred until all solids dissolved to afford a clear viscous liquid. The clear viscous liquid was filtered via 0.2 μm filter. This aqueous formulation reduced the solubility of tofacitinib citrate to less than 1 mg/mL.

Entries 3 and 4: Tofacitinib citrate aqueous formulation prepared as exemplified by entry 1 was modified by adjusting the pH to 4 and 7 to evaluate the effect of pH on the solubility of tofacitinib citrate. It was found that lowering the pH improved solubility profile of tofacitinib citrate.

Entry 5: Tofacitinib citrate (1.20 g) was added to a Carbopol solution to prepare 10 mL aqueous formulation. This mixture was stirred until all solids dissolved to afford a clear viscous liquid. The clear viscous liquid was filtered via 0.2 μm filter. The addition of 0.1% w/w carbopol 971P did not improve the solubility of tofacitinib citrate compared to aqueous formulation without the stabilizer (entry 1).

Entries 6-10: Tofacitinib citrate (1.20 g) was added to a Carbopol solution to prepare 10 mL aqueous formulation. Optionally for some aqueous formulations, 6 μL TEA or 80 μL 1 N NaOH was added to the Carbopol solution before adding tofacitinib citrate to the Carbopol solution (entries 8 and 9) or after adding tofacitinib citrate to the Carbopol solution (entry 10). The addition of 0.1% or 0.5% w/w Carbopol 971P (entries 5-10) did not improve the solubility of tofacitinib citrate compared to aqueous formulation without the stabilizer (entry 1) and produced aqueous formulations with significant un-dissolved solids. Further, the formulations with 0.5% w/w Carbopol 971P were not filterable with 0.2 μm filter.

TABLE 1

| | | | | Final Tofacitinib |
|---|---|---|---|---|
| | Excipient(s) | | Final | Citrate |
| | Carbopol | | formulation | concentration |
| | 971P | Other | pH | (mg/mL) |
| 1 | 0% | 0% | — | 3.9 |
| | | (water only) | | |
| 2 | 0% | 50 mM Citrate | — | <0.9 |
| | | buffer, pH 4 | | |
| 3 | 0% | 0% | 4 | 1.79 |
| 4 | 0% | 0% | 7 | 0.90 |
| 5 | 0.1% | 0% | — | 2.7 |
| 6 | 0.5% | 0% | — | ≤2.4 |
| 7 | 0.5% | 0% | — | 2.2 |
| 8 | 0.5% | 6 μL TEA | ~7 | 1.6 |
| 9 | 0.5% | 80 μL 1N NaOH | 4.7 | 1.7 |
| 10 | 0.5% | 80 μL 1N NaOH | 4.8 | 1.2 |

### Example 2. Evaluation of Effect of Cyclodextrin and pH on Drug Solubility

The aqueous formulations listed in Table 2 were prepared by the following method:

Tofacitinib citrate (1.20 g) was added to solution of HPβCD (10 mL). This heterogeneous mixture was briefly stirred to fully wet the solids, NaOH (10 N, 0.63 g) was added and the mixture was stirred overnight at ambient temperature. The formulation was then optionally adjusted to a particular pH using HCl (aq.). The clear viscous liquid was filtered via 0.2 μm filter.

TABLE 2

| | HPβCD | Final | Final Tofacitinib |
|---|---|---|---|
| | concentration | formulation | Citrate concentration |
| | (w/w) | pH | (mg/mL) |
| 11 | 5% HPβCD | 4 | 2.82 |
| 12 | 5% HPβCD | 7 | 3.24 |
| 13 | 10% HPβCD | 4 | 3.72 |
| 14 | 10% HPβCD | 7 | 9.41 |
| 15 | 10% HPβCD | 5.1 | 3.9† |
| 16 | 10% HPβCD | — | 7.7 (heated to 40° C. for 1 hr) |
| 17 | 10% HPβCD | — | 7.8 (heated to 40° C. for 1 hr) |
| 18 | 10% HPβCD | — (3.6) | 4.9† (not heated) |
| 19 | 20% HPβCD | 4 | 5.59 |
| 20 | 20% HPβCD | 7 | 10.43 |
| 21 | 25% HPβCD | — (3.6) | 7.7† |
| 22 | 25% HPβCD | 5.1 | 27.7† |
| 23 | 30% HPβCD | 4 | 7.50 |
| 24 | 30% HPβCD | 7 | 10.93 |
| 25 | 40% HPβCD | 4 | 10.04 |
| 26 | 40% HPβCD | 7 | 14.02 |
| 27 | 40% HPβCD | 4.5 | 12.2 |
| 28 | 40% HPβCD | — | 7.8 |
| 29 | 40% HPβCD | — (3.6) | 10.0† |
| 30 | 40% HPβCD | 5.1 | 24.5† |
| 31 | 55% HPβCD | — (3.7) | 11.5† |
| 32 | 55% HPβCD | 4.9 | 47.1† |

†Formulations were centrifuged for 5 min at 10000 RPM to spin down any solids prior to aliquoting for analyses.

Increasing concentration of HPβCD in the aqueous formulation increased the solubility of tofacitinib citrate as illustrated in Tables 1 and 2. This effect of HPβCD on solubility of tofacitinib citrate was also dependent on pH in addition to the concentration of HPβCD (Table 2).

### Example 3. Evaluation of Effect of Stabilizer in Combination with Cyclodextrin on Drug Solubility

The aqueous formulations listed in Table 3 were prepared by the following method and compared with aqueous formulations listed in Table 2 which were formulated without a stabilizer:

Hydroxypropyl-β-cyclodextrin (55.0 g) was added to a Carbopol solution (60.5 g, 2.5% mucilage). This mixture was stirred until all solids dissolved to afford a clear viscous liquid. Tofacitinib citrate (8.0 g) was added in one portion and the mixture was stirred to wet the solids. This solution was cooled in an ice bath and NaOH (10 N, 4.19 g) was added then stirred overnight at ambient temperature. The formulation was then optionally adjusted to a specific pH using HCl (aq.).

TABLE 3

| | HPβCD | Carbopol | Final | Final Tofacitinib |
|---|---|---|---|---|
| | concentration | 971P | formulation | Citrate concentration |
| | (w/w) | (w/w) | pH | (mg/mL) |
| 15 | 10% HPβCD | 0.0% | 5.1 | 3.9† |
| 33 | 10% HPβCD | 0.1% | 5.1 | 4.6† |
| 34 | 10% HPβCD | 0.5% | 5.1 | 19.7† |
| 35 | 10% HPβCD | 1.5% | — | NA* |
| 22 | 25% HPβCD | 0.0% | 5.1 | 27.7† |
| 36 | 25% HPβCD | 0.1% | 5.1 | 27.9† |
| 37 | 25% HPβCD | 0.5% | 5.0 | 25.1† |
| 38 | 25% HPβCD | 1.5% | — | NA* |
| 28 | 40% HPβCD | 0.0% | — | 7.8 |
| 39 | 40% HPβCD | 0.1% | 5.1 | 54.2† |
| 40 | 40% HPβCD | 0.5% | 5.1 | 50.3† |
| 41 | 40% HPβCD | 0.5% | — | 11.7** |
| 42 | 40% HPβCD | 1.5% | 5 | NA* |
| 43 | 40% HPβCD | 1.5% | 4.9 | 42.2† |
| 32 | 55% HPβCD | 0.0% | 4.9 | 47.1† |
| 44 | 55% HPβCD | 0.1% | 5.1 | 51.7† |
| 45 | 55% HPβCD | 0.5% | 5.1 | 51.1† |
| 46 | 55% HPβCD | 1.5% | 5.0 | 45.3† |

†Formulations were centrifuged for 5 min at 10000 RPM to spin down any solids prior to aliquoting for analyses.
*The solubility was not analyzed due to high viscosity of the aqueous formulation and sample prep challenges.
**The aqueous formulation contained some un-dissolved solids

### Example 4. Evaluation of Effect of Different Types of Cyclodextrin on Drug Solubility

The aqueous formulations listed in Table 4 were prepared by the following method:

Tofacitinib citrate (1.20 g) was added to a solution of HPCD (40% w/w, 10 mL). This heterogeneous mixture was briefly stirred to fully wet the solids, NaOH (10 N, 0.63 g) was added and the mixture was stirred overnight at ambient temperature.

TABLE 4

| | HPCD type | Final Tofacitinib Citrate concentration (mg/mL) |
|---|---|---|
| 29 | 40% HPβCD | 10.0† |
| 47 | 40% HPαCD | 5.3 |
| 48 | 40% HPγCD | 6.2 |

†Formulations were centrifuged for 5 min at 10000 RPM to spin down any solids prior to aliquoting for analyses.

Solubility of the active drug as described above can depend upon several properties of the cyclodextrin used to enhance its solubility—the inner cavity diameter of CD, number of hydrogen bond donors and acceptors on CD, solubility of the CD itself, log P of the CD, and effect of CD on the surface tension of water.

### Example 5. Evaluation of Effect of Additional Excipients on Drug Solubility

In pharmaceutical products, various excipients, such as antioxidants, antimicrobial agents, surfactants and polymers can enhance or hamper the CD solubilization of drug. The excipients in the pharmaceutical formulations containing CD may increase or decrease the ability of CD to solubilize drugs depending on their nature and physicochemical properties.

As shown in Example 3, the effect of addition of excipient to a formulation comprising an active drug and cyclodextrin cannot be predicted without experimentation. The effect of various stabilizers at varied concentrations were evaluated to investigate if they can cause an enhancement of CD's solubilization of drug.

The aqueous formulations listed in Table 5 were prepared by the following method:

Aqueous preparations comprising tofacitinib citrate and Carbopol 971P were prepared as illustrated in Example 3. Various excipients were then added to the preparation and the mixture was stirred for 24-48 hours and then assayed. The pH was adjusted to 5 where indicated.

TABLE 5

| | HPβCD | Carbopol | | Final | Final Tofacitinib |
|---|---|---|---|---|---|
| | concentration | 971P | | formulation | Citrate concentration |
| | (w/w) | (w/w) | Excipient | pH | (mg/mL) |
| 28 | 40% HPβCD | 0.0% | 0.0% | — | 7.8 |
| 49 | 40% HPβCD | 0% | 0.5% poloxamer | — | 11* |
| | | | 188 (v/v) | | |
| 50 | 40% HPβCD | 0% | 10% PEG 200 | — | 6.3 |
| | | | (v/v) | | |
| 46 | 55% HPβCD | 1.5% | 0.0% | 5.0 | 45.3† |
| 51 | 55% HPβCD | 1.5% | 10% PEG 200 | 5 | 60 |
| | | | (v/v) | | |
| 52 | 55% HPβCD | 1.5% | 20% PEG 200 | — | 60 |
| | | | (v/v) | | |
| 40 | 40% HPβCD | 0.5% | 0.0% | 5.1 | 50.3† |
| 53 | 40% HPβCD | 0.5% | 1% benzyl alcohol | 5 | 40 |
| | | | (v/v) | | |
| 54 | 40% HPβCD | 1.0% | 1% benzyl alcohol | 5 | 40 |
| | | | (v/v) | | |

†Formulations were centrifuged for 5 min at 10000 RPM to spin down any solids prior to aliquoting for analyses.
*un-dissolved solids were present in the formulation

For aqueous formulations of tofacitinib citrate comprising 55% HPβCD and 1.5% Carbopol 971P, increasing concentration of PEG from 10% v/v PEG 200 to 20% v/v PEG 200 maintained the drug solubility but provided a hazy solution.

For the aqueous formulation of tofacitinib citrate comprising 40% HPβCD and 0.5% Carbopol 971P, addition of 1% benzyl alcohol (v/v) formed solution which had a cloudy or opaque appearance before centrifugation. Similarly, for the aqueous formulation of tofacitinib citrate comprising 40% HPβCD and 1.0% Carbopol 971P, addition of 1% benzyl alcohol (v/v) also formed solution which has a slightly hazy appearance before centrifugation.

### Example 6. Evaluation of Effect of Use of Salt or Free Base Form of Active Drug on Drug Solubility

The aqueous formulations listed in Table 6 were prepared by the following method:

Entry 55: Tofacitinib (1.20 g) was added to a 40% w/w HPβCD solution to prepare 10 mL aqueous formulation. The aqueous formulation was heated at 60° C. for 1 hour before analyzing it for solubility.

Entry 56: Tofacitinib (1.20 g) was added to a 0.5% w/w Carbopol 971P solution to prepare 10 mL aqueous formulation. The aqueous formulation results into a suspension with a milky appearance and thus was not further analyzed.

Entry 57: Hydroxypropyl-p-cyclodextrin was added to a Carbopol solution in required amounts to prepare 10 mL solution. This mixture was stirred until all solids dissolved to afford a clear viscous liquid. Tofacitinib (8.0 g) was added in one portion and the mixture was stirred to wet the solids. This solution was cooled in an ice bath and NaOH (10 N, 4.19 g) was added then stirred overnight at ambient temperature. The aqueous formulation formulated was an extremely viscous suspension of un-dissolved solids and was further subjected to centrifugation or filtration.

Entry 58: The formulation was prepared as illustrated in entry 57. TEA was used to adjust the pH to 4.9. The aqueous formulation formulated was an extremely viscous suspension of un-dissolved solids and was further subjected to centrifugation or filtration.

TABLE 6

| | Excipient(s) | | | | |
|---|---|---|---|---|---|
| | HPβCD | Carbopol | | Final | Final Tofacitinib |
| | concentration | 971P | | formulation | Citrate concentration |
| | (w/w) | (w/w) | Excipient | pH | (mg/mL) |
| 55 | 40% | 0% | 0% | — | 7.8 |
| 56 | 0% | 0.5% | 0% | 7 | NA* |
| 57 | 40% | 0.5% | 0% | — | 11.9 |
| 58 | 40% | 0.5% | TEA | 4.9 | 9.0 |

†Formulations were centrifuged for 5 min at 10000 RPM to spin down any solids prior to aliquoting for analyses.
*The formulation appeared as a milky suspension and the tofacitinib solubility was determined.

### Example 7. Evaluation of Effect of SBEβCD on Drug Solubility

Tofacitinib citrate (1.20 g) was added to solution with appropriate concentration of SBEβCD (10 mL). This heterogeneous mixture was briefly stirred to fully wet the solids, NaOH (10 N, 0.63 g) was added and the mixture was stirred overnight at ambient temperature. The formulation was then adjusted to a particular pH using HCl (aq.) or 1 N NaOH.

TABLE 7

| | SBEβCD | Final | Final Tofacitinib |
|---|---|---|---|
| | concentration | formulation | Citrate concentration |
| | (w/w) | pH | (mg/mL) |
| 59 | 5% | 4 | 4.52 |
| 60 | 5% | 7 | 4.43 |
| 61 | 10% | 4 | 6.00 |
| 62 | 10% | 7 | 10.49 |
| 63 | 20% | 4 | 9.01 |
| 64 | 20% | 7 | 12.53 |
| 65 | 30% | 4 | 11.85 |
| 66 | 30% | 7 | 14.12 |
| 67 | 40% | 4 | 14.94 |
| 68 | 40% | 7 | 17.31 |

Table 7 shows the tofacitinib citrate solubility profile for formulations prepared using SBEβCD.

### Example 8. Evaluation of Effect of MβCD on Drug Solubility

The aqueous formulations listed in Table 8 were prepared by the following method:

Tofacitinib citrate (1.20 g) was added to solution with appropriate concentration of MβCD (10 mL). This heterogeneous mixture was briefly stirred to fully wet the solids, NaOH (10 N, 0.63 g) was added and the mixture was stirred overnight at ambient temperature. The formulation was then adjusted to a particular pH using HCl (aq.) or 1 N NaOH.

TABLE 8

| | MβCD | Final | Final Tofacitinib |
|---|---|---|---|
| | concentration | formulation | Citrate concentration |
| | (w/w) | pH | (mg/mL) |
| 69 | 39% | 5.0 | 21.8 |
| 70 | 56% | — (5.5) | 110.6 |
| 71 | 56% | 5.0 | 82.6 |
| 72 | 69% | — (5.7) | 84.5 |
| 73 | 69% | 5.0 | 84.5 |

Table 8 show the tofacitinib citrate solubility profile for formulations prepared using MβCD.

### Example 9. pH Range Finding with TRIS

In order to determine the optimal pH for the formulations described herein, pH range finding experiments were performed. It was determined that a pH range of 6.0-8.0, and most preferably a pH value of about 6.5, for tofacitinib citrate in 62% w/w HPβCD and 2.75:1 (TRIS:API molar equivalence) yielded the most stable formulation.

The initial step in trying to stabilize the formulation for a long-term stability was to determine what pH range the API can stay in solution for before precipitation. From data gathered, the API is soluble in high concentration HPβCD vehicle at an approximate pH of 6 or above. The initial step in determining the optimal pH was addition of hydrochloric acid and/or sodium hydroxide solutions to solubilize the API based as well as incorporating the use of stabilizers/additives (meglumine, TRIS, Carbopol, or HPMC).

Based on analytical results of formulation development, it was determined that the lower pH range below 6 has a visible effect of precipitating the API over time (ranging from complete insolubility to solubilized with gradual slow precipitation). However, if the pH enters the “high” range of 8 or above, the formulation showed more accelerated degradation on the reverse-phase HPLC results, despite desirable visual appearance (clear/transparent homogenous solution). Additionally, some formulations in the higher pH range formed an emulsion and/or two immiscible layers within the solution. Based on HPLC analyses of the two separate layers, the top liquid layer showed a significant reduction of tofacitinib citrate concentration, thus the high pH range was not pursued due to time-dependent non-homogeneity and accelerated API degradation.

Once the optimal pH range was determined (approximately 6-8), the control (no additive/stabilizer) was pH-adjusted to homogeneity and analyzed over time. The results showed undesirable degradation profiles and pH changes for many formulations despite the initial pH adjustments. Upon investigation, the control formulations saw varying degrees of degradation due to the pH adjustment process itself. pH adjustment contributed to degradation under certain circumstances due to the solution being viscous and each addition of acid or base creating a localized spike in pH at the site of acid/base addition in the solution. As a result, pH adjustment with acid and base was removed, and was replaced with stabilizers/additives instead to achieve stability within the optimal pH range.

Each stabilizer/additive previously mentioned was added in varying molar equivalent concentration with respect to the API and analyzed via pH, visual inspection, and HPLC. It was concluded that the 2.75:1 (TRIS:API) performed the best out of the stabilizer candidates and yielded a pH of 6.5 once the solution was completely homogenous. It must be noted that in this case tris/tromethamine was not used as buffering agent nor for pH adjustment, but moreso as an additive.

### Example 10. Stability Determination

Formulation stability of the high concentration tofacitinib citrate in 6200 w/w HPβCD and 2.75:1 (TRIS:API molar equivalent) is shown in the table below. As part of formulation development, stability analysis was initiated to assess solution stability at 5° C., ambient, controlled room temperature (CRT, 25° C./65% Relative Humidity), and 40° C./75% RH. Based on this study, the high concentration API formulation was determined to be stable in 5° C. for at least one-year.

| | 2.75:1 TRIS:API Formulation: 75% w/v HPBCD Stock, 100 mg/mL API | | | |
|---|---|---|---|---|
| | 0.2 um Filtered | | | |
| | | By Assay | | By Related |
| | Test Method: | | Concentration | Substance |
| Time (Month) | Condition | % Purity | (mg/mL) | % Purity* |
| 0 | N/A | 99.88 | 93.3 | — |
| 1 | 5 C. | 99.90 | 84.8 | — |
| | Ambient | 99.78 | 96.9 | — |
| | 25 C./65% RH | 99.81 | 93.4 | — |
| | 40 C./75% RH | 98.65 | 93.0 | — |
| 2 | 5 C. | 99.87 | 91.9 | — |
| | Ambient | 99.66 | 91.3 | — |
| | 25 C./65% RH | 99.54 | 92.7 | — |
| | 40 C./75% RH | 97.34 | 79.4 | — |
| 3 | 5 C. | 99.79 | 91.9 | — |
| | Ambient | 99.24 | 91.2 | — |
| | 25 C./65% RH | 99.34 | 90.5 | — |
| | 40 C./75% RH | 95.84 | 84.6 | — |
| 4 | 5 C. | 99.87 | 92.4 | — |
| | Ambient | 99.27 | 90.8 | — |
| | 25 C./65% RH | 99.33 | 90.7 | — |
| | 40 C./75% RH | 94.73 | 72.8 | — |
| 5 | 5 C. | 99.87 | 92.5 | — |
| | Ambient | 99.09 | 91.1 | — |
| | 25 C./65% RH | 99.08 | 92.1 | — |
| | 40 C./75% RH | 93.37 | 75.5 | — |
| 6 | 5 C. | 99.81 | 90.2 | — |
| | Ambient | 98.90 | 89.7 | — |
| | 25 C./65% RH | 98.86 | 87.0 | — |
| | 40 C./75% RH | 90.57 | 57.3 | — |
| 9 | 5 C. | 99.77 | 96.9 | 99.69 |
| | Ambient | 98.07 | 86.4 | 98.38 |
| | 25 C./65% RH | 98.31 | 79.3 | 98.20 |
| 12 | 5 C. | 99.72 | 93.0 | 99.38 |
| | Ambient | 98.01 | 80.4 | 97.78 |
| | 25 C./65% RH | 97.29 | 77.0 | 96.99 |

*Related substance results not available for T = 0 to T = 6M because the method was not yet developed during that time frame.

What is claimed is:

1. An aqueous formulation comprising:
tofacitinib or a pharmaceutically acceptable salt thereof; and
cyclodextrin at a concentration of at least about 10% w/w;
wherein, after extended period of time under storage condition, at least about 75% of the tofacitinib or a pharmaceutically acceptable salt remains in the aqueous formulation; or
wherein the tofacitinib or a pharmaceutically acceptable salt thereof is present in the aqueous formulation at a concentration of at least about 5 mg/mL.

2. The aqueous formulation of claim 1, wherein at least about 90% of the tofacitinib or a pharmaceutically acceptable salt remains in the aqueous formulation after extended period of time under storage condition.

3. The aqueous formulation of claim 1, wherein at least about 95% of the tofacitinib or a pharmaceutically acceptable salt remains in the aqueous formulation after extended period of time under storage condition.

4. The aqueous formulation of claim 1, wherein the extended period of time is about 3 months.

5. The aqueous formulation of claim 1, wherein the extended period of time is about 12 months.

6. The aqueous formulation of claim 1, wherein the storage condition has a storage temperature of about 2-8° C.

7. The aqueous formulation of claim 1, wherein the storage condition has a storage temperature of about 25° C.

8. The aqueous formulation of claim 1, wherein the tofacitinib salt is selected from the group consisting of tofacitinib citrate, oxalate, malate, tartrate and acetate.

9. The aqueous formulation of claim 1, wherein the tofacitinib salt is tofacitinib citrate.

10. The aqueous formulation of claim 1, further comprising a stabilizer selected from the group consisting of tromethamine (TRIS), glycine, a polyacrylic acid polymer, and combinations thereof.

11. The aqueous formulation of claim 10, wherein the polyacrylic acid polymer is a Carbopol®.

12. The aqueous formulation of claim 10, wherein the stabilize is TRIS, and wherein the TRIS: tofacitinib molar ratio is 3.0:1 to 2.0:1.

13. The aqueous formulation of claim 1, wherein the cyclodextrin concentration is at least about 10%, about 15%, about 20%, about 25%, about 30%, about 35%, about 40%, about 45%, about 50%, about 55% about 60%, about 65%, about 70%, about 75%, about 80%, about 85%, or about 90%.

14. The aqueous formulation of claim 1, wherein the cyclodextrin is α-cyclodextrin, β-cyclodextrin or γ-cyclodextrin; or a derivative thereof.

15. The aqueous formulation of claim 1, wherein the cyclodextrin is β-cyclodextrin; or a derivative thereof.

16. The aqueous formulation of claim 15, wherein the β-cyclodextrin or derivative thereof is hydroxypropyl-β-cyclodextrin, sulfobutylether β-cyclodextrin or methyl-beta-cyclodextrin.

17. The aqueous formulation of claim 1, wherein the cyclodextrin is hydroxypropyl-β-cyclodextrin.

18. The aqueous formulation of claim 17, wherein the hydroxypropyl-β-cyclodextrin is present at a concentration ranging from about 60% to about 65% (w/w).

19. The aqueous formulation of claim 1, wherein the cyclodextrin is sulfobutylether-p-cyclodextrin.

20. The aqueous formulation of claim 19, wherein the sulfobutylether-β-cyclodextrin is present at a concentration ranging from about 10% to about 40% (w/w).

21. The aqueous formulation of claim 1, wherein the cyclodextrin is methyl-β-cyclodextrin.

22. The aqueous formulation of claim 21, wherein the methyl-β-cyclodextrin is present at a concentration ranging from about 35% to about 70% (w/w).

23. The aqueous formulation of claim 1, wherein pH of the aqueous formulation ranges from about pH 4.0 to about pH 8.

24. The aqueous formulation of claim 1, wherein pH of the aqueous formulation is from about pH 6 to about pH 7.

25. The aqueous formulation of claim 1, wherein the tofacitinib or a pharmaceutically acceptable salt is present in the aqueous formulation at a concentration of from about 10 mg/mL to about 140 mg/mL.

26. The aqueous formulation of claim 1, comprising:
tofacitinib citrate at a concentration of about 10 mg/mL to about 140 mg/mL;
cyclodextrin at a concentration of about 40% w/w to about 80% w/w; and
tromethamine (TRIS) at a molar ratio of 3.5:1 to 1.5:1 (TRIS: tofacitinib).

27. The aqueous formulation of claim 1, comprising:
tofacitinib citrate at a concentration of about 10 mg/mL to about 130 mg/mL;
hydroxypropyl-β-cyclodextrin at a concentration of about 10% w/w to about 55% w/w; and
Carbopol® 971P at a concentration ranging from about 0.05% w/w to about 1.5% w/w, wherein the pH of the aqueous formulation ranges from about pH 4.0 to about pH 8.

28. The aqueous formulation of claim 1, comprising:
tofacitinib citrate at a concentration of about 10 mg/mL to about 150 mg/mL;
sulfobutylether β-cyclodextrin at a concentration of about 10% w/w to about 40% w/w; and
Carbopol® 971 at a concentration ranging from about 0.05% w/w to about 1.5% w/w, wherein the pH of the aqueous formulation ranges from about pH 4.0 to about pH 8.

29. The aqueous formulation of claim 1, comprising:
tofacitinib citrate at a concentration of about 20 mg/mL to about 140 mg/mL and methyl-β-cyclodextrin at a concentration of about 35% w/w to about 70% w/w, wherein the pH of the aqueous formulation ranges from about pH 4.0 to about pH 8.

30. The aqueous formulation of claim 1, in combination with an ingestible device configured to topically deliver the aqueous formulation to gastrointestinal tract of a subject.
